SEQUENCE LISTING (70 sequences)

SEQ ID NO: 1 - protein, 26 aa, Artificial Sequence
  synthetic 26mer peptide
FLQPQQPFPQQPQQPYPQQPQQPFPQ

SEQ ID NO: 2 - protein, 33 aa, Artificial Sequence
  synthetic 33-mer peptide
LQLQPFPQPQLPYPQPQLPYPQPQLPYPQPQPF

SEQ ID NO: 3 - DNA, 1790 bp, Magnaporthe oryzae
atgctgttcctttcttctctccttctcctggccctgtccggggctccggcctacgcagtccgcgtcggcaaccttttggagccgcctatgcccccgccctttgccatcgaggatatcgaggatatagaccccaagcaacttaccaagcgtaagatcagcagcgggttctttgatcaatacatcgaccacagcaatccttcattgggcacgtttcggcagaagttttggtggagtgatgagttctacaagggtccaggctctcctgtgattctgttcaacccaggagaatcaagggccgatatctacaccggctacctgacgaaccttaccgttcccggcatgtatgcgcaggctgttggtgccgccgtcgtcatgctcgagcaccgctactggggagagtcgtcacctttcgcaaacctcagcaccaagaacatgcagtatctgaccctcaacaactccatctccgatacaactcgctttgcccgccaggtgaagctgccttttgacaccagcggggcgaccaatgcccccaatgctccgtgggtctttgttggtggttcataccctggtgcccttgccggatgggtagagagcgtcgcccctggaactttctgggcctaccatgcgtcaagcgccgtggtccaggatatcggtgattactggcgctactttagcccaattaatgaaggcatgcccaagaactgcagcgccgacatcggccgggtcgtcgagcacattgacaaagtattgggcactggatcagacagcgacaagtctgccctgcagacagcttttggccttggatccctcgagcatgatgactttgtcgagactctggcgaacggcccatacctgtggcagggcattgatttcagcactggctactcggactttttcaagttctgtgactatgttgaggtatgctcgctatcgcctatcctctttgttaaaattggcaggaaactgtggcgcagtaatttgctgacttcatcctctagaacgtacccccgaaagcagcgacaagagtgcccccaggagttgatggtgttggtcttgagaaggcattgacgggctaccaggattggatcaagaaggaatacctcccaaccgcctgcgacagcttgggataccccaagggtgacctgggctgcctgagcagccacaacttctcagcccccttctaccgtgaccagacagtattaaacccggggaaccggcagtggttttggtttctttgcaatgaaccgtgagtggcgacggcagtgggctttgatttaaacttactactgtcttcttttgtactgacacgagtttgcccatccttcagcttcaagttttggcaaaacggcgcccccaagggcgagccgtcgattgtttcgcgtatcataggcagcaaatactttgagagccagtgtgcgttgtggttccccgacgagccgcgtgaaggcggtggcgtttacacgtacggcatcgccgagggcaaggatgtcgccagtgtcaacaaattcaccggtgggtgggaccacaccgacacgaaacgacttctttgggtcaacggccagtttgacccatggctgcacgctacagtgtcgtcgccctcccgccccggaggtccccttcaatcgacagacaaggcacctgttctggttatcccgggtggagtacactgcaccgacttgattatacgcaacggagacgccaacgagggcgcgcgcaaggtccagagtcaggcacgcgaaatcatcaagaaatgggtgtccgagtttcccaagagcggaaagagcccttga

SEQ ID NO: 4 - protein, 542 aa, Magnaporthe oryzae
MLFLSSLLLLALSGAPAYAVRVGNLLEPPMPPPFAIEDIEDIDPKQLTKRKISSGFFDQYIDHSNPSLGTFRQKFWWSDEFYKGPGSPVILFNPGESRADIYTGYLTNLTVPGMYAQAVGAAVVMLEHRYWGESSPFANLSTKNMQYLTLNNSISDTTRFARQVKLPFDTSGATNAPNAPWVFVGGSYPGALAGWVESVAPGTFWAYHASSAVVQDIGDYWRYFSPINEGMPKNCSADIGRVVEHIDKVLGTGSDSDKSALQTAFGLGSLEHDDFVETLANGPYLWQGIDFSTGYSDFFKFCDYVENVPPKAATRVPPGVDGVGLEKALTGYQDWIKKEYLPTACDSLGYPKGDLGCLSSHNFSAPFYRDQTVLNPGNRQWFWFLCNEPFKFWQNGAPKGEPSIVSRIIGSKYFESQCALWFPDEPREGGGVYTYGIAEGKDVASVNKFTGGWDHTDTKRLLWVNGQFDPWLHATVSSPSRPGGPLQSTDKAPVLVIPGGVHCTDLIIRNGDANEGARKVQSQAREIIKKWVSEFPKSGKSP

SEQ ID NO: 5 - DNA, 1822 bp, Aspergillus flavus
atggtgtccctcacgcatatattttcgaaggccctcctcacactgctggtgggccagtctgctgccctaagctttctccccggcatcaaggccaataatctccaactcgcctcggtattaggtatcgatggccataccgccaggttcaatcctgagaagatcgcagagaccgctatctcgcgcggttctggctcagaagtccctgcccggcggatatcggtatgtctttaccagtcaagctttctagtatatgaggtaaaatctaactcggcgttcagatccccattgaccatgaggatccatctatgggcacctatcagaaccgctactgggtttcagcagacttttacaagcccggtggccccgtctttgtactagatgccggtgaaggcaatgcctactccgtggcgcaatcgtatctcggcggatcggataacttcttcgcggagtacctcaaggaattcaatgggctgggccttgtgtgggagcatcggtgagccacctaccctagtcatcattgtcatgattgaccgctaacctccggtccgattgaagttactatggtgactctctgcccttccctgtcaacactagcacccccaacgagcatttcaagtacctcaccaacagccaggcactggctgacctcccttacttcgctgagaagttcactctcaacgggacagacttgagccccaagtccagtccctggatcatgctcggtggctcatacccgggcatgcgcgcggccttcacccgcaacgagtacccggacaccattttcgcctcgttcgccatgtctgcgcccgtcgaagcctgggtcaacatgaccatctacttcgagcaagtctaccgcggcatggttgcgaacggactgggcggctgtgccaaggacctcaaggccatcaacgactacatcgacagccaactcgacaagaagggccaagccgccgacgccatcaagacactcttcctcggtaaagaaggcatccacaactccaacggcgacttcaccgccgcgctcggaagcatctacaacctcttccagagctacggcgtcgacggcggcgaagaaagtctctcccagctctgcagctacctcgacaaagaagccagccccaacggcatcgcccggaaaatcggagtcaaggaactgaccgagaagttcgccgcctggcccccgcttctgtacctcatcaaccagtggggcagccaggtcggtaacggcgactccaactgcaagggccagaacaattccaccgagaccgtctgtgagctgggcgggcagttcaccgaccccgacaccatcagctggacctggcagtactgcaccgaatggggctatctccaggccgacaacgtgggccctcactccctactctccaagtaccagtccctggagtaccagcagtccctttgctaccgacagttccccggcgcaaaggagagtggcctgctccccgagcacccggaggcgaacgagacgaacgccgaaacaggcggatggaccatccgtccttccaatgtcttctggagcgcgggcgagttcgatccctggcggacgttgacgcccttgtcgaatgagacattcgcgccgaagggcgtgcagatctccaccaatatccccaagtgtggtgtcgagacacctgagaatgtgctcttcggctatgtcattccgagggcggagcattgctttgactatgacttgagttacaagccggctgataagtcgcggaagttgttcagtcttgccttgaagaagtggctcccgtgctggcggtcggagcatgctcctaagggtgtacagaggaagtggatgtaa

SEQ ID NO: 6 - protein, 566 aa, Aspergillus flavus
MVSLTHIFSKALLTLLVGQSAALSFLPGIKANNLQLASVLGIDGHTARFNPEKIAETAISRGSGSEVPARRISIPIDHEDPSMGTYQNRYWVSADFYKPGGPVFVLDAGEGNAYSVAQSYLGGSDNFFAEYLKEFNGLGLVWEHRYYGDSLPFPVNTSTPNEHFKYLTNSQALADLPYFAEKFTLNGTDLSPKSSPWIMLGGSYPGMRAAFTRNEYPDTIFASFAMSAPVEAWVNMTIYFEQVYRGMVANGLGGCAKDLKAINDYIDSQLDKKGQAADAIKTLFLGKEGIHNSNGDFTAALGSIYNLFQSYGVDGGEESLSQLCSYLDKEASPNGIARKIGVKELTEKFAAWPPLLYLINQWGSQVGNGDSNCKGQNNSTETVCELGGQFTDPDTISWTWQYCTEWGYLQADNVGPHSLLSKYQSLEYQQSLCYRQFPGAKESGLLPEHPEANETNAETGGWTIRPSNVFWSAGEFDPWRTLTPLSNETFAPKGVQISTNIPKCGVETPENVLFGYVIPRAEHCFDYDLSYKPADKSRKLFSLALKKWLPCWRSEHAPKGVQRKWM

SEQ ID NO: 7 - DNA, 1773 bp, Coccidioides posadasii
atgaggtttctccaaaacctactcgggggcactgctttggcactgcttacaggccttgggtcggcctttggaccaagatgggcacgctatcagaacgaccttcacctagctgcaatgctaggtatggatgctgattctgtcttgaccaaccgaagcagcctcgcctctgccattgacagtcttgccgagacatccgctgtggtcgctgaatacgcaaatgtacgccatccccatagctcctttgggtgtgctctgtcttaattcctgaaatagattcctatcgatcacagaaaccctggaagaatgtacaggaatcgatactgggtgaacgatcaatattatcagccaggagggcctgtggttattttcgataccggtgagaccaatggtcaagcttttgccgattattatttggtcgatcctacgtcctacattgtccaattgcttcgggaatttcatggcgtaggccttgtttgggagcaccggtatgaagtcaatttctactaataggaacggataggaggctaactttatggaagatattatggcgaatctctcccttaccccgtcaatgggcagacgtctgctgcgcaattccaatacttgacgctcgaacaagctttgcaggatctcccttactttgccagaacatttcgccgacctcggctccctaatgctgatctgacaccaagatcaaccccgtggattatggtcggcggctcatacccaggcatgcgtgcagctttctcgagactcaagtatcccgacactatttttgctgccttttcctcttctgcacctgctcaagctaggattgacatgagcgtttattatgagcaggtgtacaggggtttggtagcatatggctatggaaattgcaccagggacgtcaatgctgcataccgatatattgatgcccaacttgccaaccccagtaccgctgctcaaattaagagacaattcttaggtcccggtgccgagcaaaacagcaatggcgattttactgcagttttgctctataattgggcgacatggcagagctttggggcaaatggccctgcgggtcagttctgtaattggctcgaaacagaccaatatggcagagtggcccctgctgaaggctgggcaccttcaagaggtgctagatctgtggtcgacagatgggctgcatggccgggactcagccgagcgatcaactccatttttgaaacaaactgcaattgcccagaagagacttgctcctgtgacctttctgcgccacctgcagaccccctggccatcagctggtcgtggcagttttgctcgcagttcggttacttccagtaccagaatcccagaccccatgagatcgcttcgcgctatcaaacggaagcttacatccaagataactgctaccggcagttccctgacggcgtgagcagcggccatcttccccgccgccctcgagccgatgcgacaaacaattatactggaggctggaacatgcgcccttcaaacgtcttccacggcgctggacaatacgacccgtggactcctttgactgtgctttcccaggagccttggggaccacgccgtcgcgtcaccactcaaatcccggcgtgcaatcaggaacaagaggcggtttttggcgtcctgcttcccaatgcagagcacgtttacgatcttcaaacctcttaccaaccgggcgaggtatccaggcaactgttcagaagggcgctgcaccagtggcttccttgcttccggaggaggaattcaacggcagatcatgattga

SEQ ID NO: 8 - protein, 554 aa, Coccidioides posadasii
MRFLQNLLGGTALALLTGLGSAFGPRWARYQNDLHLAAMLGMDADSVLTNRSSLASAIDSLAETSAVVAEYANIPIDHRNPGRMYRNRYWVNDQYYQPGGPVVIFDTGETNGQAFADYYLVDPTSYIVQLLREFHGVGLVWEHRYYGESLPYPVNGQTSAAQFQYLTLEQALQDLPYFARTFRRPRLPNADLTPRSTPWIMVGGSYPGMRAAFSRLKYPDTIFAAFSSSAPAQARIDMSVYYEQVYRGLVAYGYGNCTRDVNAAYRYIDAQLANPSTAAQIKRQFLGPGAEQNSNGDFTAVLLYNWATWQSFGANGPAGQFCNWLETDQYGRVAPAEGWAPSRGARSVVDRWAAWPGLSRAINSIFETNCNCPEETCSCDLSAPPADPLAISWSWQFCSQFGYFQYQNPRPHEIASRYQTEAYIQDNCYRQFPDGVSSGHLPRRPRADATNNYTGGWNMRPSNVFHGAGQYDPWTPLTVLSQEPWGPRRRVTTQIPACNQEQEAVFGVLLPNAEHVYDLQTSYQPGEVSRQLFRRALHQWLPCFRRRNSTADHD

SEQ ID NO: 9 - protein, 523 aa, Magnaporthe oryzae
VRVGNLLEPPMPPPFAIEDIEDIDPKQLTKRKISSGFFDQYIDHSNPSLGTFRQKFWWSDEFYKGPGSPVILFNPGESRADIYTGYLTNLTVPGMYAQAVGAAVVMLEHRYWGESSPFANLSTKNMQYLTLNNSISDTTRFARQVKLPFDTSGATNAPNAPWVFVGGSYPGALAGWVESVAPGTFWAYHASSAVVQDIGDYWRYFSPINEGMPKNCSADIGRVVEHIDKVLGTGSDSDKSALQTAFGLGSLEHDDFVETLANGPYLWQGIDFSTGYSDFFKFCDYVENVPPKAATRVPPGVDGVGLEKALTGYQDWIKKEYLPTACDSLGYPKGDLGCLSSHNFSAPFYRDQTVLNPGNRQWFWFLCNEPFKFWQNGAPKGEPSIVSRIIGSKYFESQCALWFPDEPREGGGVYTYGIAEGKDVASVNKFTGGWDHTDTKRLLWVNGQFDPWLHATVSSPSRPGGPLQSTDKAPVLVIPGGVHCTDLIIRNGDANEGARKVQSQAREIIKKWVSEFPKSGKSP

SEQ ID NO: 10 - protein, 544 aa, Aspergillus flavus
LSFLPGIKANNLQLASVLGIDGHTARFNPEKIAETAISRGSGSEVPARRISIPIDHEDPSMGTYQNRYWVSADFYKPGGPVFVLDAGEGNAYSVAQSYLGGSDNFFAEYLKEFNGLGLVWEHRYYGDSLPFPVNTSTPNEHFKYLTNSQALADLPYFAEKFTLNGTDLSPKSSPWIMLGGSYPGMRAAFTRNEYPDTIFASFAMSAPVEAWVNMTIYFEQVYRGMVANGLGGCAKDLKAINDYIDSQLDKKGQAADAIKTLFLGKEGIHNSNGDFTAALGSIYNLFQSYGVDGGEESLSQLCSYLDKEASPNGIARKIGVKELTEKFAAWPPLLYLINQWGSQVGNGDSNCKGQNNSTETVCELGGQFTDPDTISWTWQYCTEWGYLQADNVGPHSLLSKYQSLEYQQSLCYRQFPGAKESGLLPEHPEANETNAETGGWTIRPSNVFWSAGEFDPWRTLTPLSNETFAPKGVQISTNIPKCGVETPENVLFGYVIPRAEHCFDYDLSYKPADKSRKLFSLALKKWLPCWRSEHAPKGVQRKWM

SEQ ID NO: 11 - protein, 532 aa, Coccidioides posadasii
FGPRWARYQNDLHLAAMLGMDADSVLTNRSSLASAIDSLAETSAVVAEYANIPIDHRNPGRMYRNRYWVNDQYYQPGGPVVIFDTGETNGQAFADYYLVDPTSYIVQLLREFHGVGLVWEHRYYGESLPYPVNGQTSAAQFQYLTLEQALQDLPYFARTFRRPRLPNADLTPRSTPWIMVGGSYPGMRAAFSRLKYPDTIFAAFSSSAPAQARIDMSVYYEQVYRGLVAYGYGNCTRDVNAAYRYIDAQLANPSTAAQIKRQFLGPGAEQNSNGDFTAVLLYNWATWQSFGANGPAGQFCNWLETDQYGRVAPAEGWAPSRGARSVVDRWAAWPGLSRAINSIFETNCNCPEETCSCDLSAPPADPLAISWSWQFCSQFGYFQYQNPRPHEIASRYQTEAYIQDNCYRQFPDGVSSGHLPRRPRADATNNYTGGWNMRPSNVFHGAGQYDPWTPLTVLSQEPWGPRRRVTTQIPACNQEQEAVFGVLLPNAEHVYDLQTSYQPGEVSRQLFRRALHQWLPCFRRRNSTADHD

SEQ ID NO: 12 - DNA, 1629 bp, Artificial Sequence
  synthesized nucleotide sequence encoding full-length MorPro1
atgctctttctgagctccctcctgctgctcgctctcagcggcgctcccgcctacgccgttcgagttggcaacctcctggagcctcccatgcctcctccctttgctattgaggacatcgaagacattgaccctaagcagctcaccaagcgaaaaatcagcagcggtttcttcgaccagtacatcgaccactccaaccccagcctcggtactttccgccaaaagttttggtggtccgacgagttctacaagggccccggttcccccgtcatcctgttcaaccctggcgaaagccgcgctgatatctacaccggctatctgactaacctcaccgtccccggcatgtacgctcaagccgtcggtgctgccgttgtcatgctggagcaccgctattggggcgagtccagccccttcgccaatctctccaccaagaacatgcagtacctgaccctcaacaacagcattagcgacaccacccgctttgcccgccaggtcaagctgccctttgacacctccggcgccaccaatgctcctaatgccccctgggtctttgtcggtggtagctatcctggtgccctggccggttgggtcgagagcgttgctcctggcaccttctgggcctatcatgccagctccgccgtcgttcaagatatcggcgactattggcgctactttagccccatcaacgagggcatgcctaaaaactgcagcgccgacatcggtcgcgtcgtcgaacacatcgataaggtcctgggtaccggctccgacagcgataagagcgccctgcagaccgctttcggcctcggcagcctggaacacgacgacttcgtcgagaccctcgccaacggcccctacctctggcagggcatcgacttcagcactggctacagcgacttcttcaagttctgcgactacgtcgagaatgtccctcccaaggccgccactcgcgttcctcccggcgtcgacggcgtcggcctggagaaggccctgaccggttaccaggactggatcaagaaggagtacctccccaccgcctgcgattccctcggctaccccaaaggcgatctcggttgcctcagctcccacaacttctccgcccctttctaccgcgatcagaccgtcctcaaccccggtaatcgccagtggttctggttcctctgcaacgagcccttcaagttctggcaaaacggcgcccccaagggcgagcccagcatcgtcagccgcattattggcagcaagtacttcgagtcccagtgcgccctctggtttcccgatgagccccgcgagggcggcggtgtttatacttacggcatcgccgaaggtaaggatgtcgccagcgtcaataagtttactggcggctgggaccatactgacaccaaacgcctcctgtgggttaacggccagttcgacccctggctccacgccactgtcagcagccctagccgacccggtggccccctccagagcactgacaaggcccctgtcctcgttattcccggcggcgtccactgcaccgatctcatcatccgcaacggcgacgctaacgaaggcgctcgcaaggttcaaagccaggcccgcgagatcattaagaagtgggtcagcgagtttcctaaaagcggcaagtccccctaa

SEQ ID NO: 13 - DNA, 1701 bp, Artificial Sequence
  Synthesized nucleotide sequence encoding full-length AflPro3
atggtctccctcactcatattttctccaaggccctcctgactctcctggtcggtcaatccgccgctctgagcttcctccccggtatcaaggctaacaatctgcaactggcttccgtcctgggcattgacggccacaccgctcgctttaatcccgaaaaaatcgctgaaaccgccatctcccgcggttccggctccgaggttcccgctcgacgcatctccatccccatcgatcatgaggacccttccatgggcacctaccagaaccgctattgggtctccgccgatttctacaagcccggtggccccgttttcgtcctcgatgccggtgaaggcaacgcctactccgttgcccagtcctacctcggtggcagcgacaatttcttcgccgagtacctgaaggagttcaacggtctgggcctcgtctgggaacaccgatactacggcgattccctgcccttccccgtcaacacttccacccctaacgagcacttcaagtatctcaccaactcccaggctctcgccgacctcccttactttgccgaaaagtttaccctgaacggcaccgatctgtcccccaaatccagcccctggattatgctgggtggtagctatcccggcatgcgagctgctttcacccgcaatgagtaccccgataccattttcgccagcttcgccatgtccgctcccgttgaggcctgggtcaacatgactatctacttcgagcaagtctaccgcggcatggttgccaatggcctcggcggttgcgctaaggatctgaaagccatcaacgattatatcgacagccaactggacaagaaaggtcaagccgccgacgctatcaaaaccctctttctcggcaaggaaggcatccacaacagcaatggcgactttaccgccgccctcggttccatctacaacctgttccaaagctatggcgtcgacggtggcgaggaaagcctgagccagctctgcagctatctcgacaaggaggccagccctaatggcatcgcccgcaagatcggcgtcaaagagctgaccgagaagttcgccgcttggccccccctgctctacctcatcaaccagtggggctcccaagttggtaacggcgacagcaactgtaaaggccagaacaactccaccgaaactgtctgcgaactgggcggtcagttcaccgaccccgacaccatttcctggacctggcagtactgcactgaatggggctacctccaggctgataacgtcggccctcacagcctcctcagcaagtaccagagcctcgaataccagcagtccctgtgctaccgccaattccccggcgccaaggagagcggtctcctgcccgagcaccctgaggccaatgagaccaacgccgagactggtggctggaccatccgccctagcaacgtcttctggtccgccggcgaatttgatccctggcgcaccctcacccccctctccaacgagaccttcgctcctaagggcgtccagatctccaccaatatccccaagtgcggcgttgaaacccctgagaacgtcctcttcggctacgtcatcccccgagccgaacactgcttcgactacgacctgtcctacaaacccgccgacaagagccgcaaactgttcagcctcgccctgaagaagtggctgccctgttggcgcagcgagcacgcccctaaaggcgttcagcgcaagtggatgtaa

SEQ ID NO: 14 - DNA, 1665 bp, Artificial Sequence
  synthesized nucleotide sequence encoding full-length CpoPro1
atgcgctttctgcaaaatctcctgggcggcactgctctggctctcctcactggcctcggctccgcctttggtccccgctgggcccgctaccaaaacgatctccacctggccgctatgctgggcatggacgccgacagcgtcctgaccaaccgcagcagcctcgcctccgccattgattccctggctgaaacttccgccgtcgtcgccgaatacgccaacattcccatcgaccaccgaaaccccggtcgcatgtaccgcaaccgatactgggtcaacgaccaatattaccagcccggtggccctgtcgttatcttcgacaccggcgaaactaatggccaagcctttgctgactactacctcgtcgaccccacctcctatatcgtccaactcctccgcgagttccatggcgtcggcctcgtctgggagcatcgctactacggcgagagcctcccctaccccgtcaacggccagacctccgctgcccaattccaatatctcactctggagcaggccctccaagatctgccctacttcgcccgaactttccgacgaccccgcctgcctaatgccgatctcaccccccgaagcaccccctggatcatggtcggcggttcctatcctggcatgcgcgctgcttttagccgactgaagtaccccgacactatttttgccgccttcagcagctccgctcccgctcaggcccgcattgacatgagcgtctactacgagcaggtttatcgcggcctggtcgcttatggttacggcaactgcactcgcgacgttaatgctgcctaccgctacattgacgcccagctcgccaaccctagcactgccgctcaaatcaaacgccaatttctcggtcccggtgccgagcagaatagcaacggcgacttcactgctgtcctgctctacaactgggccacttggcaatcctttggcgctaatggtcctgccggccagttttgtaactggctggagaccgaccagtacggtcgagtcgcccctgccgaaggctgggctccttcccgcggtgctcgatccgttgtcgaccgatgggctgcctggcccggtctgtcccgcgctattaactccatttttgagactaattgtaattgtcccgaagagacctgtagctgcgacctcagcgcccctcctgctgaccctctggccatcagctggagctggcagttctgcagccaattcggctacttccagtaccagaatcctcgcccccacgagatcgctagccgataccagactgaggcttatatccaagacaattgctaccgacagttccccgacggcgttagctccggtcacctgccccgccgccctcgagccgatgccactaacaactacactggcggctggaacatgcgccccagcaatgtctttcacggcgctggtcagtatgacccttggactcccctcaccgtcctgtcccaggaaccttggggccctcgccgccgagtcaccactcagatccccgcctgcaatcaagaacaggaggccgtcttcggtgttctcctccccaacgccgaacacgtttacgacctgcagaccagctatcaacctggtgaggtcagccgacaactgtttcgacgcgccctgcatcagtggctgccctgctttcgacgccgcaactccaccgctgatcatgactaa

SEQ ID NO: 15 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 16 - protein, 590 aa, Trichoderma reesei
SVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 17 - protein, 578 aa, Aspergillus oryzae
EAFEKLSAVPKGWHYSSTPKGNTEVCLKIALAQKDAAGFEKTVLEMSDPDHPSYGQHFTTHDEMKRMLLPRDDTVDAVRQWLENGGVTDFTQDADWINFCTTVDTANKLLNAQFKWYVSDVKHIRRLRTLQYDVPESVTPHINTIQPTTRFGKISPKKAVTHSKPSQLDVTALAAAVVAKNISHCDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLALGN

SEQ ID NO: 18 - protein, 574 aa, Phaeosphaeria nodorum
EPFEKLFSTPEGWKMQGLATNEQIVKLQIALQQGDVAGFEQHVIDISTPSHPSYGAHYGSHEEMKRMIQPSSETVASVSAWLKAAGINDAEIDSDWVTFKTTVGVANKMLDTKFAWYVSEEAKPRKVLRTLEYSVPDDVAEHINLIQPTTRFAAIRQNHEVAHEIVGLQFAALANNTVNCDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAAVPSRYRA

SEQ ID NO: 19 - protein, 590 aa, Trichoderma atroviride
NAAVLLDSLDKVPVGWQAASAPAPSSKITLQVALTQQNIDQLESKLAAVSTPNSSNYGKYLDVDEINQIFAPSSASTAAVESWLKSYGVDYKVQGSSIWFQTDVSTANKMLSTNFHTYTDSVGAKKVRTLQYSVPETLADHIDLISPTTYFGTSKAMRALKIQNAASAVSPLAARQEPSSCKGTIEFENRTFNVFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELVRY

SEQ ID NO: 20 - protein, 580 aa, Arthroderma benhamiae
KPTPGASHKVIEHLDFVPEGWQMVGAADPAAIIDFWLAIERENPEKLYDTIYDVSTPGRAQYGKHLKREELDDLLRPRAETSESIINWLTNGGVNPQHIRDEGDWVRFSTNVKTAETLMNTRFNVFKDNLNSVSKIRTLEYSVPVAISAHVQMIQPTTLFGRQKPQNSLILNPLTKDLESMSVEEFAASQCRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVLP

SEQ ID NO: 21 - protein, 580 aa, Fusarium graminearum
KSYSHHAEAPKGWKVDDTARVASTGKQQVFSIALTMQNVDQLESKLLDLSSPDSKNYGQWMSQKDVTTAFYPSKEAVSSVTKWLKSKGVKHYNVNGGFIDFALDVKGANALLDSDYQYYTKEGQTKLRTLSYSIPDDVAEHVQFVDPSTNFGGTLAFAPVTHPSRTLTERKNKPTKSTVDASCQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLSF

SEQ ID NO: 22 - protein, 603 aa, Acremonium alcalophilum
AVVIRAAVLPDAVKLMGKAMPDDIISLQFSLKQQNIDQLETRLRAVSDPSSPEYGQYMSESEVNEFFKPRDDSFAEVIDWVAASGFQDIHLTPQAAAINLAATVETADQLLGANFSWFDVDGTRKLRTLEYTIPDRLADHVDLISPTTYFGRARLDGPRETPTRLDKRQRDPVADKAYFHLKWDRGTSNCDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGLALGRGT

SEQ ID NO: 23 - protein, 614 aa, Sodiomyces alkalinus
AVVIRAAPLPESVKLVRKAAAEDGINLQLSLKRQNMDQLEKFLRAVSDPFSPKYGQYMSDAEVHEIFRPTEDSFDQVIDWLTKSGFGNLHITPQAAAINVATTVETADQLFGANFSWFDVDGTPKLRTGEYTIPDRLVEHVDLVSPTTYFGRMRPPPRGDGVNDWITENSPEQPAPLNKRDTKTESDQARDHPSWDSRTPDCATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELALED

SEQ ID NO: 24 - protein, 575 aa, Aspergillus kawachii
VVHEKLAAVPSGWHHLEDAGSDHQISLSIALARKNLDQLESKLKDLSTPGESQYGQWLDQEEVDTLFPVASDKAVISWLRSANITHIARQGSLVNFATTVDKVNKLLNTTFAYYQRGSSQRLRTTEYSIPDDLVDSIDLISPTTFFGKEKTSAGLTQRSQKVDNHVAKRSNSSSCADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLSL

SEQ ID NO: 25 - protein, 582 aa, Talaromyces stipitatus
AAALVGHESLAALPVGWDKVSTPAAGTNIQLSVALALQNIEQLEDHLKSVSTPGSASYGQYLDSDGIAAQYGPSDASVEAVTNWLKEAGVTDIYNNGQSIHFATSVSKANSLLGADFNYYSDGSATKLRTLAYSVPSDLKEAIDLVSPTTYFGKTTASRSIQAYKNKRASTTSKSGSSSVQVSASCQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLSF

SEQ ID NO: 26 - protein, 579 aa, Fusarium oxysporum
KSFSHHAEAPQGWQVQKTAKVASNTQHVFSLALTMQNVDQLESKLLDLSSPDSANYGNWLSHDELTSTFSPSKEAVASVTKWLKSKGIKHYKVNGAFIDFAADVEKANTLLGGDYQYYTKDGQTKLRTLSYSIPDDVAGHVQFVDPSTNFGGTVAFNPVPHPSRTLQERKVSPSKSTVDASCQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLSL

SEQ ID NO: 27 - protein, 590 aa, Trichoderma virens
SVLVESLEKLPHGWKAASAPSPSSQITLQVALTQQNIDQLESRLAAVSTPNSKTYGNYLDLDEINEIFAPSDASSAAVESWLHSHGVTKYTKQGSSIWFQTEVSTANAMLSTNFHTYSDAAGVKKLRTLQYSIPESLVGHVDLISPTTYFGTSNAMRALRSKSVASVAQSVAARQEPSSCKGTLVFEGRTFNVFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELIRYI

SEQ ID NO: 28 - protein, 569 aa, Trichoderma atroviride
AVLVESLKQVPNGWNAVSTPDPSTSIVLQIALAQQNIDELEWRLAAVSTPNSGNYGKYLDIGEIEGIFAPSNASYKAVASWLQSHGVKNFVKQAGSIWFYTTVSTANKMLSTDFKHYSDPVGIEKLRTLQYSIPEELVGHVDLISPTTYFGNNHPATARTPNMKAINVTYQIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALSV

SEQ ID NO: 29 - protein, 565 aa, Agaricus bisporus
SPLARRWDDFAEKHAWVEVPRGWEMVSEAPSDHTFDLRIGVKSSGMEQLIENLMQTSDPTHSRYGQHLSKEELHDFVQPHPDSTGAVEAWLEDFGISDDFIDRTGSGNWVTVRVSVAQAERMLGTKYNVYRHSESGESVVRTMSYSLPSELHSHIDVVAPTTYFGTMKSMRVTSFLQPEIEPVDPSAKPSAAPASCLSTTVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQGLI

SEQ ID NO: 30 - protein, 583 aa, Magnaporthe oryzae
RVFDSLPHPPRGWSYSHAAESTEPLTLRIALRQQNAAALEQVVLQVSNPRHANYGQHLTRDELRSYTAPTPRAVRSVTSWLVDNGVDDYTVEHDWVTLRTTVGAADRLLGADFAWYAGPGETLQLRTLSYGVDDSVAPHVDLVQPTTRFGGPVGQASHIFKQDDFDEQQLKTLSVGFQVMADLPANGPGSIKAACNESGVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLAVGAPGVPNA

SEQ ID NO: 31 - protein, 594 aa, Togninia minima
SDVVLESLREVPQGWKRLRDADPEQSIKLRIALEQPNLDLFEQTLYDISSPDHPKYGQHLKSHELRDIMAPREESTAAVIAWLQDAGLSGSQIEDDSDWINIQTTVAQANDMLNTTFGLFAQEGTEVNRIRALAYSVPEEIVPHVKMIAPIIRFGQLRPQMSHIFSHEKVEETPSIGTIKAAAIPSVDLNVTACNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNLSTPNFHLPHIGGH

SEQ ID NO: 32 - protein, 595 aa, Bipolaris maydi
STTSHVEGEVVERLHGVPEGWSQVGAPNPDQKLRFRIAVRSADSELFERTLMEVSSPSHPRYGQHLKRHELKDLIKPRAKSTSNILNWLQESGIEARDIQNDGEWISFYAPVKRAEQMMSTTFKTYQNEARANIKKIRSLDYSVPKHIRDDIDIIQPTTRFGQIQPERSQVFSQEEVPFSALVVNATCNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLATAPKSGERRVRRGGLGGQA

SEQ ID NO: 33 - protein, 613 aa, Aspergillus kawachii
MLSSFLSQGAAVSLALLSLLPSPVAAEIFEKLSGVPNGWRYANNPHGNEVIRLQIALQQHDVAGFEQAVMDMSTPGHADYGKHFRTHDEMKRMLLPSDTAVDSVRDWLESAGVHNIQVDADWVKFHTTVNKANALLDADFKWYVSEAKHIRRLRTLQYSIPDALVSHINMIQPTTRFGQIQPNRATMRSKPKHADETFLTAATLAQNTSHCDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGVALGEAKAYGN

SEQ ID NO: 34 - protein, 658 aa, Aspergillus nidulans
MAATGRFTAFWNVASVPALIGILPLAGSHLRAVLCPVCIWRHSKAVCAPDTLQAMRAFTRVTAISLAGFSCFAAAAAAAFESLRAVPDGWIYESTPDPNQPLRLRIALKQHNVAGFEQALLDMSTPGHSSYGQHFGSYHEMKQLLLPTEEASSSVRDWLSAAGVEFEQDADWINFRTTVDQANALLDADFLWYTTTGSTGNPTRILRTLSYSVPSELAGYVNMIQPTTRFGGTHANRATVRAKPIFLETNRQLINAISSGSLEHCEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVLDA

SEQ ID NO: 35 - protein, 604 aa, Aspergillus ruber
MLSFVRRGALSLALVSLLTSSVAAEVFEKLHVVPEGWRYASTPNPKQPIRLQIALQQHDVTGFEQSLLEMSTPDHPNYGKHFRTHDEMKRMLLPNENAVHAVREWLQDAGISDIEEDADWVRFHTTVDQANDLLDANFLWYAHKSHRNTARLRTLEYSIPDSIAPQVNVIQPTTRFGQIRANRATHSSKPKGGLDELAISQAATADDDSICDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLALSA

SEQ ID NO: 36 - protein, 600 aa, Aspergillus terreus
MLPSLVNNGALSLAVLSLLTSSVAGEVFEKLSAVPKGWHFSHAAQADAPINLKIALKQHDVEGFEQALLDMSTPGHENYGKHFHEHDEMKRMLLPSDSAVDAVQTWLTSAGITDYDLDADWINLRTTVEHANALLDTQFGWYENEVRHITRLRTLQYSIPETVAAHINMVQPTTRFGQIRPDRATFHAHHTSDARILSALAAASNSTSCDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELAP

SEQ ID NO: 37 - protein, 601 aa, Penicillium digitatum
MIASLFNRRALTLALLSLFASSATADVFESLSAVPQGWRYSRTPSANQPLKLQIALAQGDVAGFEAAVIDMSTPDHPSYGNHFNTHEEMKRMLQPSAESVDSIRNWLESAGISKIEQDADWMTFYTTVKTANELLAANFQFYINGVKKIERLRTLKYSVPDALVSHINMIQPTTRFGQLRAQRAILHTEVKDNDEAFRSNAMSANPDCNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLANAE

SEQ ID NO: 38 - protein, 601 aa, Penicillium oxalicum
MHVPLLNQGALSLAVVSLLASTVSAEVFDKLVAVPEGWRFSRTPSGDQPIRLQVALTQGDVEGFEKAVLDMSTPDHPNYGKHFKSHEEVKRMLQPAGESVEAIHQWLEKAGITHIQQDADWMTFYTTVEKANNLLDANFQYYLNENKQVERLRTLEYSVPDELVSHINLVTPTTRFGQLHAEGVTLHGKSKDVDEQFRQAATSPSSDCNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAVKQK

SEQ ID NO: 39 - protein, 601 aa, Penicillium roqueforti
MIASLFSRGALSLAVLSLLASSAAADVFESLSAVPQGWRYSRRPRADQPLKLQIALTQGDTAGFEEAVMEMSTPDHPSYGHHFTTHEEMKRMLQPSAESAESIRDWLEGAGITRIEQDADWMTFYTTVETANELLAANFQFYVSNVRHIERLRTLKYSVPKALVPHINMIQPTTRFGQLRAHRGILHGQVKESDEAFRSNAVSAQPDCNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLANAE

SEQ ID NO: 40 - protein, 601 aa, Penicillium rubens
MIASLFNRGALSLAVLSLLASSASADVFESLSAVPQGWRYSRRPRADQPLKLQIALAQGDTAGFEEAVMDMSTPDHPSYGNHFHTHEEMKRMLQPSAESADSIRDWLESAGINRIEQDADWMTFYTTVETANELLAANFQFYANSAKHIERLRTLQYSVPEALMPHINMIQPTTRFGQLRVQGAILHTQVKETDEAFRSNAVSTSPDCNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLANGE

SEQ ID NO: 41 - protein, 594 aa, Neosartorya fischeri
MLSSTLYAGLLCSLAAPALGVVHEKLSAVPSGWTLVEDASESDTTTLSIALARQNLDQLESKLTTLATPGNAEYGKWLDQSDIESLFPTASDDAVIQWLKDAGVTQVSRQGSLVNFATTVGTANKLFDTKFSYYRNGASQKLRTTQYSIPDSLTESIDLIAPTVFFGKEQDSALPPHAVKLPALPRRAATNSSCANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 42 - protein, 568 aa, Aspergillus fumigatus
MLSSTLYAGWLLSLAAPALCVVQEKLSAVPSGWTLIEDASESDTITLSIALARQNLDQLESKLTTLATPGNPEYGKWLDQSDIESLFPTASDDAVLQWLKAAGITQVSRQGSLVNFATTVGTANKLFDTKFSYYRNGASQKLRTTQYSIPDHLTESIDLIAPTVFFGKEQNSALSSHAVKLPALPRRAATNSSCANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 43 - protein, 415 aa, Trichoderma reesei
QEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 44 - protein, 391 aa, Aspergillus oryzae
CDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLA

SEQ ID NO: 45 - protein, 388 aa, Phaeosphaeria nodorum
CDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAA

SEQ ID NO: 46 - protein, 395 aa, Trichoderma atroviride
VFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELV

SEQ ID NO: 47 - protein, 389 aa, Arthroderma benhamiae
CRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVL

SEQ ID NO: 48 - protein, 397 aa, Fusarium graminearum
CQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLS

SEQ ID NO: 49 - protein, 408 aa, Acremonium alcalophilum
CDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGL

SEQ ID NO: 50 - protein, 410 aa, Sodiomyces alkalinus
CATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELA

SEQ ID NO: 51 - protein, 400 aa, Aspergillus kawachii
CADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLS

SEQ ID NO: 52 - protein, 396 aa, Talaromyces stipitatus
CQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLS

SEQ ID NO: 53 - protein, 397 aa, Fusarium oxysporum
CQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLS

SEQ ID NO: 54 - protein, 395 aa, Trichoderma virens
VFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELI

SEQ ID NO: 55 - protein, 398 aa, Trichoderma atroviride
QIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALS

SEQ ID NO: 56 - protein, 363 aa, Agaricus bisporus
TVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQ

SEQ ID NO: 57 - protein, 376 aa, Magnaporthe oryzae
GVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLA

SEQ ID NO: 58 - protein, 388 aa, Togninia minima
CNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNL

SEQ ID NO: 59 - protein, 390 aa, Bipolaris maydis
CNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLAT

SEQ ID NO: 60 - protein, 393 aa, Aspergillus kawachii
CDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGV

SEQ ID NO: 61 - protein, 392 aa, Aspergillus nidulans
CEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVL

SEQ ID NO: 62 - protein, 392 aa, Aspergillus ruber
CDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLAL

SEQ ID NO: 63 - protein, 391 aa, Aspergillus terreus
CDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELA

SEQ ID NO: 64 - protein, 391 aa, Penicillium digitatum
CNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLA

SEQ ID NO: 65 - protein, 391 aa, Penicillium oxalicum
CNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAV

SEQ ID NO: 66 - protein, 391 aa, Penicillium roqueforti
CNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLA

SEQ ID NO: 67 - protein, 391 aa, Penicillium rubens
CNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLA

SEQ ID NO: 68 - protein, 400 aa, Neosartorya fischeri
CANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 69 - protein, 374 aa, Aspergillus fumigatus
CANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 70 - protein, 4 aa, Artificial Sequence
  substrate amino acid sequence
AAAP

(19) United States
(12) Patent Application Publication
(10) Pub. No.: US 2021/0315238 A1
(43) Pub. Date: Oct. 14, 2021

(54) PROLINE SPECIFIC ENDOPEPTIDASES

(71) Applicant: DUPONT NUTRITION BIOSCIENCES APS, Copenhagen K (DK)

(72) Inventors: Xiaogang GU, Copenhagen K (DK); Karsten Matthias KRAGH, Copenhagen K (DK); Ernest MEINJOHANNS, Copenhagen K (DK); Xinyue TANG, Copenhagen K (DK)

(21) Appl. No.: 17/264,547

(22) PCT Filed: Jul. 31, 2019
(86) PCT No.: PCT/EP2019/070694

§ 371 Date: Jan. 29, 2021

(30) Foreign Application Priority Data
Jul. 31, 2018 (CN) PCT/CN2018/097762

(51) Int. Cl. A23L 5/20; A23L 33/17; C12N 9/48; C12N 9/52

(52) U.S. Cl. CPC A23L 5/25; A23L 33/17; C12N 9/485; C12Y 304/16006; C12Y 304/14002; C12Y 304/16005; C12N 9/52

(57) ABSTRACT

The present invention provides proline specific endopeptidases. The present invention further provides methods for use of proline specific endopeptidases for use in reduction of chill haze in a beverage, including beer. The present invention further provides methods for producing protein hydrolysates using proline specific endopeptidases. Also provided are methods of treating disease, including Celiac disease using proline specific endopeptidases. Also provided are nucleic acids encoding the proline specific endopeptidases and host cells for production of the proline specific endopeptidases.

### TECHNICAL FIELD

The present invention relates to proline specific endopeptidases. More particularly, the present invention relates to the use of proline specific endopeptidases for reduction or elimination of beer haze, production of protein hydrolysates and detoxification of gluten proteins, including amelioration of gluten intolerance and Celiac disease.

### BACKGROUND

Beer-haze, a cloudy appearance in beer, is caused by the aggregation of hydrophobic proteins, e.g. hordeins from barley, and polyphenols, resulting in a beer with an undesirable cloudy appearance or haze. See, e.g., Asano, K.; Shinagawa, K.; Hashimoto, N. Characterization of haze-forming proteins of beer and their roles in chill haze formation. J. Am. Soc. Brew. Chem. 1982, 40, 147-154. The same phenomenon is also called chill-haze and similar haze formation may also occur in wine and fruit juices.

It has been suggested that acid proteases such as papain can be used to degrade beer proteins and hence prevent haze formation. However, broad spectrum proteases such as papain have been found to impair beer foam formation and stability. See, e.g, Posada, J.; Almenar, J.; Garcia Galindo, J. A practical approach on protein stabilizers. Proc.-Eur. Brew. Conv. 1971, 13, 379-391. For this reason, more selective proteases such as proline specific endopeptidases have been employed to reduce beer haze. However, there is a continuing need for proteases that can be used to reduce beer-haze, because present commercial offerings are overly expensive and do not provide complete beer-haze removal. Moreover, there is a concern that some of the current beer haze proteases survive the brewing process and are present in the final beer product. Hence, there is also a need for proteases that can be used to prevent chill haze but which do not survive the brewing process.

Celiac disease, also known as gluten-sensitive enteropathy, is a widespread, autoimmune disease of the small intestine induced in patients having susceptible genetic backgrounds by the intake of gluten proteins from common sources such as wheat, rye and barley. In susceptible patients, exposure of the small intestine to gluten induces a CD4+ T cell mediated immune response. Celiac disease normally appears in early childhood and includes such severe symptoms as chronic diarrhea, fatigue, weight loss, and abdominal distension. Left untreated, Celiac disease increases the risk of infertility, bone disorders and intestinal malignancies.

Gluten is a composite of storage proteins found in many cereal grains such as wheat, rye, oats, barley, maize and rice. Recent molecular and genetic studies have strongly implicated gliadin proteins as the immunogenic component of wheat gluten. A 33-mer peptide from alpha-2 gliadin and a 26-mer peptide from gamma-gliadin have been identified as the primary initiators of the inflammatory response of gluten in Celiac Sprue patients (Shan et al., (2005) J Proteome Res., 4(5): 1732-1741). Both 26-mer and 33-mer peptides contain multiple copies of antigenic epitopes. They are very rich in proline and reported to be resistant to pepsin, trypsin and chymotrypsin (Bethune and Khosla, (2012) Methods Enzymol, 502: 241-271).

There is a continuing need for proteases that are capable of degrading the immunogenic proline rich protein sequences in wheat gliadins and similar proteins from barley, rye, oats and maize.

### SUMMARY OF THE INVENTION

In accordance with an aspect of the instant invention, an isolated polypeptide is described having proline specific endopeptidase activity having a polypeptide which is at least 70% sequence identity to one of SEQ ID NO: 4, SEQ ID NO: 6 and SEQ ID NO: 8 or a fragment thereof. Optionally, the polypeptide has at least 80% sequence identity to one of SEQ ID NO: 4, SEQ ID NO: 6 and SEQ ID NO: 8 or a fragment thereof. Optionally, the polypeptide has at least 90% sequence identity to one of SEQ ID NO: 4, SEQ ID NO: 6 and SEQ ID NO: 8 or a fragment thereof. Optionally, the polypeptide has at least 95% sequence identity to one of SEQ ID NO: 4, SEQ ID NO:6 and SEQ ID NO:8 or a fragment thereof. Optionally, the polypeptide has at least 99% sequence identity to one of SEQ ID NO: 4, SEQ ID NO:6 and SEQ ID NO:8. Optionally, the polypeptide is a sequence according to one of SEQ ID NO:4, SEQ ID NO:6 and SEQ ID NO:8 or a fragment thereof.

In accordance with an aspect of the present invention, a method for the reduction or prevention of haze in a beverage is presented having the step of adding an isolated polypeptide having proline specific endopeptidase as described above to the beverage. Optionally, the beverage contains at least one protein. Optionally, the protein comprises hordein. Optionally, the beverage further comprises polyphenols. Optionally, the beverage has a pH of less than 7.

Optionally, the beverage is a fruit juice. Optionally, the beverage is a wine. Optionally, the beverage is a beer. Optionally, the isolated polypeptide is added to a mash.

Optionally, the isolated polypeptide is added before haze formation. Optionally, the isolated polypeptide is added after haze formation.

Optionally, the method of haze reduction has the further step of adding a second isolated polypeptide having proline specific endopeptidase activity as described above wherein the second isolated polypeptide is different than the isolated polypeptide. Optionally, the isolated polypeptide is a polypeptide according to SEQ ID NO:4 or a fragment thereof and the second isolated polypeptide is a polypeptide according to SEQ ID NO:8 or a fragment thereof.

In another aspect of the present invention, a method for forming a protein hydrolysate is presented having the step of adding to a protein substrate an isolated polypeptide having endopeptidase as described above. Optionally, the method includes the further step of adding a protease wherein the protease is different than the isolated polypeptide. Optionally, the protease is selected from the group consisting of a serine protease, a cysteine protease, an endopeptidase, and an exopeptidase.

Optionally, the protease is a serine protease. Optionally, the serine protease is a subtilisin.

Optionally, the protease is an endopeptidase.

Optionally, the endopeptidase is a second isolated polypeptide having proline specific endopeptidase activity as described above. Optionally, the isolated polypeptide is a polypeptide according to SEQ ID NO:4 or a fragment thereof and the second isolated polypeptide is a polypeptide according to SEQ ID NO:8 or a fragment thereof.

Preferably, the protease is an exopeptidase. Optionally, the exopeptidase is a tripeptidyl aminopeptidase. Optionally, the tripeptidyl aminopeptidase is a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the polypeptide has at least 70% sequence identity to SEQ ID NO:15, SEQ ID NO:16 or SEQ ID NO:17 or a fragment thereof.

Optionally, the polypeptide has at least 80% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the polypeptide has at least 80% sequence identity to SEQ ID NO:15, SEQ ID NO:16 or SEQ ID NO:17 or a fragment thereof.

Optionally, the polypeptide has at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the polypeptide has at least 90% sequence identity to SEQ ID NO:15, SEQ ID NO:16 or SEQ ID NO:17 or a fragment thereof.

Optionally, the polypeptide has at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the polypeptide has at least 90% sequence identity to SEQ ID NO:15, SEQ ID NO:16 or SEQ ID NO:17 or a fragment thereof.

Optionally, the polypeptide has at least 99% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase has at least 99% sequence identity to SEQ ID NO:15, SEQ ID NO:16 or SEQ ID NO:17 or a fragment thereof.

Optionally, the polypeptide is a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the polypeptide is a sequence as set forth in SEQ ID NO:15, SEQ ID NO:16 and SEQ ID NO:17 or a fragment thereof.

Optionally, in the method of making a hydrolysate, in addition to the isolated polypeptide having proline specific endopeptidase and the polypeptide having tripeptidyl amino peptidase activity a second isolated polypeptide having proline specific endopeptidase activity as described above is added wherein the second isolated polypeptide is different than the isolated polypeptide having proline specific endopeptidase activity.

Optionally, the isolated polypeptide is a polypeptide according to SEQ ID NO:4 or a fragment thereof and the second isolated polypeptide is a polypeptide according to SEQ ID NO:8 or a fragment thereof.

Optionally, the protein substrate is derived from milk. Optionally, the protein substrate is derived from wheat.

In another aspect of the present invention, a method for degrading gluten in food is presented having the step of contacting gluten-containing food with an isolated polypeptide having proline specific endopeptidase activity as described above.

Optionally, the food is bread or beer.

In another aspect of the present invention, a method for treating gluten intolerance, celiac disease, dermatitis herpetiformis and/or gluten sensitivity in a patient in need of such treatment, wherein the treatment reduces exposure of the patient to an immunogenic gluten peptide, having the step of orally administering to the patient a therapeutically effective dose of an isolated polypeptide having proline specific endopeptidase activity as described above contemporaneously with the ingestion of a food that may contain gluten.

In another aspect of the present invention, the use is presented of an isolated polypeptide having proline specific endopeptidase activity as described above for the manufacture of a dietary supplement or medicament.

Optionally, the isolated polypeptide having proline specific endopeptidase activity as described above digests gluten fragments that are resistant to normal digestive enzymes.

Optionally, the isolated polypeptide having proline specific endopeptidase activity as described above is admixed with food.

Optionally, the isolated polypeptide having proline specific endopeptidase activity as described above is stable to acid conditions.

In another aspect of the present invention, a formulation is presented having the isolated polypeptide having proline specific endopeptidase activity as described above and a pharmaceutically acceptable excipient.

In other aspect of the present invention, an enzyme blend is presented having a proline specific endopeptidase as described above and a protease wherein the proline specific endopeptidase is different than said protease. Optionally, the protease is selected from the group consisting of a serine protease, a cysteine protease, an endopeptidase, and an exopeptidase.

Optionally, the protease is a serine protease. Optionally, the serine protease is a subtilisin.

Optionally, the protease is an endopeptidase. Optionally, the endopeptidase is a second isolated polypeptide having proline specific endopeptidase activity as described above. Optionally, the isolated polypeptide is a polypeptide according to SEQ ID NO:4 or a fragment thereof and the second isolated polypeptide is a polypeptide according to SEQ ID NO:8 or a fragment thereof.

Optionally, the protease is an exopeptidase. Optionally, the exopeptidase is a tripeptidyl aminopeptidase. Optionally, the tripeptidyl aminopeptidase is a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof. Optionally, the polypeptide has at least 70% sequence identity to SEQ ID NO:15, SEQ ID NO:16 or SEQ ID NO:17 or a fragment thereof.

Optionally, the tripeptidyl aminopeptidase is a polypeptide having tripeptidyl aminopeptidase activity having at least 80% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase is a polypeptide having tripeptidyl aminopeptidase activity having at least 80% sequence identity to SEQ ID NO:15, SEQ ID NO:16 or SEQ ID NO:17 or a fragment thereof.

Optionally, the tripeptidyl aminopeptidase is a polypeptide having tripeptidyl aminopeptidase activity having at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase is a polypeptide having tripeptidyl aminopeptidase activity having at least 90% sequence identity to SEQ ID NO:15, SEQ ID NO:16 or SEQ ID NO:17 or a fragment thereof.

Optionally, the tripeptidyl aminopeptidase is a polypeptide having tripeptidyl aminopeptidase activity having at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase is a polypeptide having tripeptidyl aminopeptidase activity having 95% sequence identity to SEQ ID NO:15, SEQ ID NO:16 or SEQ ID NO:17 or a fragment thereof.

Optionally, the tripeptidyl aminopeptidase is a polypeptide having tripeptidyl aminopeptidase activity having at least 99% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase has at least 99% sequence identity to SEQ ID NO:15, SEQ ID NO:16 or SEQ ID NO:17 or a fragment thereof.

Optionally, the tripeptidyl aminopeptidase is a polypeptide having a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Optionally, the tripeptidyl aminopeptidase is a sequence as set forth in SEQ ID NO:15, SEQ ID NO:16 or SEQ ID NO:17 or a fragment thereof.

Optionally, where an enzyme blend has a polypeptide having proline specific endopeptidase activity as described above and a tripeptidyl aminopeptidase as described above, a second isolated polypeptide having proline specific endopeptidase activity as describe above is included in the blend wherein the second isolated polypeptide is different than the isolated polypeptide having proline specific endopeptidase activity.

According to this aspect of the present invention, the isolated polypeptide is optionally a polypeptide according to SEQ ID NO:4 or a fragment thereof and the second isolated polypeptide having proline specific endopeptidase activity is optionally a polypeptide according to SEQ ID NO:8 or a fragment thereof.

In another aspect of the present invention, a polynucleotide is presented having a nucleic acid sequence encoding the isolated polypeptide having proline specific endopeptidase activity as described above.

In another aspect of the present invention, a recombinant expression vector is presented having the polynucleotide.

In another aspect of the present invention, a host cell is presented having the recombinant expression vector.

### BRIEF DESCRIPTION OF THE BIOLOGICAL SEQUENCES

SEQ ID NO: 1 is the amino acid sequence of the synthesized 26-mer peptide discussed in the examples.

SEQ ID NO:2 is the amino acid sequence of the synthesized 33-mer peptide discussed in the examples.

SEQ ID NO: 3 is the nucleotide sequence of full-length MorPro1 gene.

SEQ ID NO: 4 is the amino acid sequence of MorPro1 precursor protein.

SEQ ID NO: 5 is the nucleotide sequence of full-length AflPro3 gene.

SEQ ID NO: 6 is the amino acid sequence of AflPro3 precursor protein.

SEQ ID NO: 7 is the nucleotide sequence of full-length CpoPro1 gene.

SEQ ID NO: 8 is the amino acid sequence of CpoPro1 precursor protein.

SEQ ID NO: 9 is the predicted, mature amino acid sequence of MorPro1, lacking the signal sequence.

SEQ ID NO: 10 is the predicted, mature amino acid sequence of AflPro3, lacking the signal sequence.

SEQ ID NO: 11 is the predicted, mature amino acid sequence of CpoPro1, lacking the signal sequence.

SEQ ID NO: 12 is the synthesized nucleotide sequence encoding full-length MorPro1.

SEQ ID NO: 13 is the synthesized nucleotide sequence encoding full-length AflPro3.

SEQ ID NO: 14 is the synthesized nucleotide sequence encoding full-length CpoPro1.

SEQ ID NO: 15 is the peptidase with leader sequence from Trichoderma reesei.

SEQ ID NO: 16 is the peptidase with no leader sequence from Trichoderma reesei.

SEQ ID NO: 17 is the peptidase from Aspergillus oryzae.

SEQ ID NO: 18 is the peptidase from Phaeosphaeria nodorum.

SEQ ID NO: 19 is the peptidase from Trichoderma atroviride.

SEQ ID NO: 20 is the peptidase from Arthroderma benhamiae.

SEQ ID NO: 21 is the peptidase from Fusarium graminearum.

SEQ ID NO: 22 is the peptidase from Acremonium alcalophilum.

SEQ ID NO: 23 is the peptidase from Sodimomyces alkalinus.

SEQ ID NO: 24 is the peptidase from Aspergillus kawachii.

SEQ ID NO: 25 is the peptidase from Talaromyces stipitatus.

SEQ ID NO: 26 is the peptidase from Fusarium oxysporum.

SEQ ID NO: 27 is the peptidase from Trichoderma virens.

SEQ ID NO: 28 is the peptidase from Trichoderma atroviride.

SEQ ID NO: 29 is the peptidase from Agaricus bisporus.

SEQ ID NO: 30 is the peptidase from Magnaporthe oryzae.

SEQ ID NO: 31 is the peptidase from Togninia minima.

SEQ ID NO: 32 is the peptidase from Bipolaris maydi.

SEQ ID NO: 33 is the peptidase from Aspergillus kawachii.

SEQ ID NO: 34 is the peptidase from Aspergillus nidulans.

SEQ ID NO: 35 is the peptidase from Aspergillus ruber.

SEQ ID NO: 36 is the peptidase from Aspergillus terreus.

SEQ ID NO: 37 is the peptidase from Penicillium digitatum.

SEQ ID NO: 38 is the peptidase from Penicillium oxalicum.

SEQ ID NO: 39 is the peptidase from Penicillium roquefortis.

SEQ ID NO: 40 is the peptidase from Penicillium rubens.

SEQ ID NO: 41 is the peptidase from Neosartorya fischeri.

SEQ ID NO: 42 is the peptidase from Aspergillus fumigatus.

SEQ ID NO: 43 is the peptidase from Trichoderma reesei.

SEQ ID NO: 44 is the peptidase from Aspergillus oryzae.

SEQ ID NO: 45 is the peptidase from Phaeosphaeria nodorum.

SEQ ID NO: 46 is the peptidase from Trichoderma atroviride.

SEQ ID NO: 47 is the peptidase from Arthroderma benhamiae.

SEQ ID NO: 48 is the peptidase from Fusarium graminearum.

SEQ ID NO: 49 is the peptidase from Acremonium alcalophilum.

SEQ ID NO: 50 is the peptidase from Sodiomyces alkalinus.

SEQ ID NO: 51 is the peptidase from Aspergillus kawachii.

SEQ ID NO: 52 is the peptidase from Talaromyces stipitatus.

SEQ ID NO: 53 is the peptidase from Fusarium oxysporum.

SEQ ID NO: 54 is the peptidase from Trichoderma virens.

SEQ ID NO: 55 is the peptidase from Trichoderma atrovirde.

SEQ ID NO: 56 is the peptidase from Agaricus bisporus.

SEQ ID NO: 57 is the peptidase from Magnaporthe oryzae.

SEQ ID NO: 58 is the peptidase from Togninia minima.

SEQ ID NO: 59 is the peptidase from Bipolaris maydis.

SEQ ID NO: 60 is the peptidase from Aspergillus kawachii.

SEQ ID NO: 61 is the peptidase from Aspergillus nidulans.

SEQ ID NO: 62 is the peptidase from Aspergillus ruber.

SEQ ID NO: 63 is the peptidase from Aspergillus terreus.

SEQ ID NO: 64 is the peptidase from Penicillium digitatum.

SEQ ID NO: 65 is the peptidase from Penicillium oxalicum.

SEQ ID NO: 66 is the peptidase from Penicillium roqueforti.

SEQ ID NO: 67 is the peptidase from Penicillium rubens.

SEQ ID NO: 68 is the peptidase from Neosartorya fischeri.

SEQ ID NO: 69 is the peptidase from Aspergillus fumigatus.

### DESCRIPTION OF FIGURES

FIG. 1 shows the plasmid map of pGX256(Trex3gM-MorPro1).

FIG. 2 shows the dose response curve of MorPro1, AflPro3 and CpoPro1.

FIG. 3 shows the pH profile of MorPro1, AflPro3 and CpoPro1.

FIG. 4 shows the temperature profile of MorPro1, AflPro3 and CpoPro1.

FIG. 5 shows the thermostability of MorPro1, AflPro3 and CpoPro1.

FIG. 6 shows the gliadin-catechin haze reduction performance of purified MorPro1 FIG. 7 shows the gliadin-catechin haze reduction performance of purified AflPro3.

FIG. 8 shows the gliadin-catechin haze reduction performance of purified CpoPro1.

### DETAILED DESCRIPTION OF THE INVENTION

### Definitions

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an endopeptidase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a proline specific endopeptidase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF. |

Deletions are counted as non-identical residues, compared to a reference sequence. Deletions occurring at either termini are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

The term “about” refers to ±5% to the referenced value.

The present proline specific endopeptidases may be “precursor,” “immature,” or “full-length,” in which case they include a signal sequence, or “mature,” in which case they lack a signal sequence. Unless otherwise noted, the amino acid residue numbering used herein refers to the mature forms of the respective endopeptidase polypeptides. The present endopeptidase polypeptides may also be truncated to remove the N or C-termini, so long as the resulting polypeptides retain endopeptidase activity.

The terms “protein”, ‘polypeptide” and “peptide” are used interchangeable herein.

A “protease” is an enzyme that breaks down proteins and polypeptides by hydrolyzing amide bonds. The term “peptidase” is used herein interchangeably with protease.

An “exopeptidase” is a protease which cleaves the terminal amino acids of a protein or polypeptide. Typically, an exopeptidase can release one, two or three amino acids from either the N- or C-terminus of a protein or polypeptide.

An “endopeptidase” is a protease which cleaves internal amide bonds within a protein or polypeptide as opposed to an exopeptidase which cleaves the terminal (e.g. 1^{st}, 2^{nd}, or 3^{rd} terminal amino acid).

A “proline specific endopeptidase” or an enzyme, protein or polypeptide having such activity cuts proteins or polypeptides at or near places near proline residues.

As used herein, “beverage” means beer, wine or fruit juice. Also, beverage as used herein includes the above beverages at all stages of their production. For example, with respect to beer, beverage also can mean a wort or malt.

“Gluten” is a composite of storage proteins found in many cereal grains such as wheat, rye, oats, barley, maize and rice.

“Celiac disease”, also known as gluten-sensitive enteropathy, is a widespread, autoimmune disease of the small intestine induced in patients having susceptible genetic backgrounds by the intake of gluten proteins from common sources such as wheat, rye and barley.

Polypeptides of the invention include full length polypeptides as described herein in for example the sequence ids and variants thereof, including fragments.

“Fragments” of the polypeptides of the instant invention are shorter sequences of the polypeptides than as described in the Sequence IDs that retain activity, e.g., proline specific endopeptidase activity. Fragments include N-terminally deleted polypeptides, C-terminally deleted polypeptides, internally deleted polypeptides or any combination(s) thereof.

“Variants” may include the deletion, modification or addition of single amino acids or groups of amino acids within the protein sequence, as long as the peptide maintains the basic biological functionality of the proline specific endopeptidases. Variants include wild type variants such as those exhibited from similar enzymes derived from other sources and those introduced using recombinant DNA technology.

Amino acid substitutions may be made, for example from 1, 2 or from 3 to 10, 20 or 30 substitutions. The modified polypeptide will generally retain activity as a proline specific endopeptidase. Conservative substitutions may be made; such substitutions are well known in the art. Preferably substitutions do not affect the folding or activity of the polypeptide.

### Production of Endopeptidases

The present proline specific endopeptidases can be produced in host cells, for example, by secretion or intracellular expression. A cultured cell material (e.g., a whole-cell broth) comprising an endopeptidase can be obtained following secretion of the endopeptidase into the cell medium. Optionally, the endopeptidase can be isolated from the host cells, or even isolated from the cell broth, depending on the desired purity of the final endopeptidase. A gene encoding a proline specific endopeptidase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, and E. Coli.

The host cell further may express a nucleic acid encoding a homologous or heterologous endopeptidase, i.e., a proline specific endopeptidase that is not the same species as the host cell, or one or more other enzymes. The endopeptidase may be a variant endopeptidase. Additionally, the host may express one or more accessory enzymes, proteins, peptides.

### Vectors

A DNA construct comprising a nucleic acid encoding a proline specific endopeptidase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding endopeptidase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a proline specific endopeptidase can be transformed and replicated in a bacterial host cell as a means of propagating and amplifying the vector. The vector also may be transformed into an expression host, so that the encoding nucleic acids can be expressed as a functional endopeptidase. Host cells that serve as expression hosts can include filamentous fungi, for example.

A nucleic acid encoding a proline specific endopeptidase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a proline specific endopeptidase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a proline specific endopeptidaseis expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the endopeptidase gene to be expressed or from a different genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant endopeptidase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol, or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of endopeptidase for subsequent enrichment or purification. Extracellular secretion of endopeptidase into the culture medium can also be used to make a cultured cell material comprising the isolated endopeptidase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the endopeptidase to a host cell organelle such as a peroxisome, or to a particular host cell compartment. Such a targeting sequence includes but is not limited to the sequence, SKL. For expression under the direction of control sequences, the nucleic acid sequence of the endopeptidase is operably linked to the control sequences in proper manner with respect to expression.

The procedures used to ligate the DNA construct encoding a proline specific endopeptidase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

### Transformation and Culture of Host Cells

An isolated cell, either comprising a DNA construct or an expression vector, is advantageously used as a host cell in the recombinant production of a proline specific endopeptidase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A proline specific endopeptidase expressed by a fungal host cell can be glycosylated, i.e., will comprise a glycosyl moiety. The glycosylation pattern can be the same or different as present in the wild-type endopeptidase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. A gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a proline specific endopeptidase is stably integrated into a host cell chromosome. Transformants are then selected and purified by known techniques.

### Expression

A method of producing a proline specific endopeptidase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a proline specific endopeptidase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a proline specific endopeptidase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the endopeptidase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding a proline specific endopeptidase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a proline specific endopeptidase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

### Methods for Enriching and Purifying Endopeptidases

Fermentation, separation, and concentration techniques are well known in the art and conventional methods can be used in order to prepare a proline specific endopeptidase polypeptide-containing solution.

After fermentation, a fermentation broth is obtained, the microbial cells and various suspended solids, including residual raw fermentation materials, are removed by conventional separation techniques in order to obtain a proline specific endopeptidase solution. Filtration, centrifugation, microfiltration, rotary vacuum drum filtration, ultrafiltration, centrifugation followed by ultra-filtration, extraction, or chromatography, or the like, are generally used.

It is desirable to concentrate a proline specific endopeptidase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary drum vacuum filtration and/or ultrafiltration.

### PREFERRED EMBODIMENTS OF THE INVENTION

| SEQ | | |
|---|---|---|
| ID | | |
| NO.: | Sequence | Origin |
| 1 | FLQPQQPFPQQPQQPYPQQPQQPFPQ | Synthetic |
| | | 26mer |
| | | peptide |
| 2 | LQLQPFPQPQLPYPQPQLPYPQPQLPYPQPQPF | Synthetic |
| | | 33mer |
| | | peptide |
| 3 | ATGCTGTTCCTTTCTTCTCTCCTTCTCCTGGCCCTGTCCGGGGCTCCGGCCTACGCAGTC | Magnaporthe |
| | CGCGTCGGCAACCTTTTGGAGCCGCCTATGCCCCCGCCCTTTGCCATCGAGGATATCGAG | oryzae |
| | GATATAGACCCCAAGCAACTTACCAAGCGTAAGATCAGCAGCGGGTTCTTTGATCAATAC | full |
| | ATCGACCACAGCAATCCTTCATTGGGCACGTTTCGGCAGAAGTTTTGGTGGAGTGATGAG | length |
| | TTCTACAAGGGTCCAGGCTCTCCTGTGATTCTGTTCAACCCAGGAGAATCAAGGGCCGAT | MorPro1 |
| | ATCTACACCGGCTACCTGACGAACCTTACCGTTCCCGGCATGTATGCGCAGGCTGTTGGT | DNA |
| | GCCGCCGTCGTCATGCTCGAGCACCGCTACTGGGGAGAGTCGTCACCTTTCGCAAACCTC | |
| | AGCACCAAGAACATGCAGTATCTGACCCTCAACAACTCCATCTCCGATACAACTCGCTTT | |
| | GCCCGCCAGGTGAAGCTGCCTTTTGACACCAGCGGGGCGACCAATGCCCCCAATGCTCCG | |
| | TGGGTCTTTGTTGGTGGTTCATACCCTGGTGCCCTTGCCGGATGGGTAGAGAGCGTCGCC | |
| | CCTGGAACTTTCTGGGCCTACCATGCGTCAAGCGCCGTGGTCCAGGATATCGGTGATTAC | |
| | TGGCGCTACTTTAGCCCAATTAATGAAGGCATGCCCAAGAACTGCAGCGCCGACATCGGC | |
| | CGGGTCGTCGAGCACATTGACAAAGTATTGGGCACTGGATCAGACAGCGACAAGTCTGCC | |
| | CTGCAGACAGCTTTTGGCCTTGGATCCCTCGAGCATGATGACTTTGTCGAGACTCTGGCG | |
| | AACGGCCCATACCTGTGGCAGGGCATTGATTTCAGCACTGGCTACTCGGACTTTTTCAAG | |
| | TTCTGTGACTATGTTGAGGTATGCTCGCTATCGCCTATCCTCTTTGTTAAAATTGGCAGG | |
| | AAACTGTGGCGCAGTAATTTGCTGACTTCATCCTCTAGAACGTACCCCCGAAAGCAGCGA | |
| | CAAGAGTGCCCCCAGGAGTTGATGGTGTTGGTCTTGAGAAGGCATTGACGGGCTACCAGG | |
| | ATTGGATCAAGAAGGAATACCTCCCAACCGCCTGCGACAGCTTGGGATACCCCAAGGGTG | |
| | ACCTGGGCTGCCTGAGCAGCCACAACTTCTCAGCCCCCTTCTACCGTGACCAGACAGTAT | |
| | TAAACCCGGGGAACCGGCAGTGGTTTTGGTTTCTTTGCAATGAACCGTGAGTGGCGACGG | |
| | CAGTGGGCTTTGATTTAAACTTACTACTGTCTTCTTTTGTACTGACACGAGTTTGCCCAT | |
| | CCTTCAGCTTCAAGTTTTGGCAAAACGGCGCCCCCAAGGGCGAGCCGTCGATTGTTTCGC | |
| | GTATCATAGGCAGCAAATACTTTGAGAGCCAGTGTGCGTTGTGGTTCCCCGACGAGCCGC | |
| | GTGAAGGCGGTGGCGTTTACACGTACGGCATCGCCGAGGGCAAGGATGTCGCCAGTGTCA | |
| | ACAAATTCACCGGTGGGTGGGACCACACCGACACGAAACGACTTCTTTGGGTCAACGGCC | |
| | AGTTTGACCCATGGCTGCACGCTACAGTGTCGTCGCCCTCCCGCCCCGGAGGTCCCCTTC | |
| | AATCGACAGACAAGGCACCTGTTCTGGTTATCCCGGGTGGAGTACACTGCACCGACTTGA | |
| | TTATACGCAACGGAGACGCCAACGAGGGCGCGCGCAAGGTCCAGAGTCAGGCACGCGAAA | |
| | TCATCAAGAAATGGGTGTCCGAGTTTCCCAAGAGCGGAAAGAGCCCTTGA | |
| 4 | MLFLSSLLLLALSGAPAYAVRVGNLLEPPMPPPFAIEDIEDIDPKQLTKRKISSGFFDQY | Magnaporthe |
| | IDHSNPSLGTFRQKFWWSDEFYKGPGSPVILFNPGESRADIYTGYLTNLTVPGMYAQAVG | oryzae |
| | AAVVMLEHRYWGESSPFANLSTKNMQYLTLNNSISDTTRFARQVKLPFDTSGATNAPNAP | full |
| | WVFVGGSYPGALAGWVESVAPGTFWAYHASSAVVQDIGDYWRYFSPINEGMPKNCSADIG | length |
| | RVVEHIDKVLGTGSDSDKSALQTAFGLGSLEHDDFVETLANGPYLWQGIDFSTGYSDFFK | MorPro1 |
| | FCDYVENVPPKAATRVPPGVDGVGLEKALTGYQDWIKKEYLPTACDSLGYPKGDLGCLSS | precursor |
| | HNFSAPFYRDQTVLNPGNRQWFWFLCNEPFKFWQNGAPKGEPSIVSRIIGSKYFESQCAL | |
| | WFPDEPREGGGVYTYGIAEGKDVASVNKFTGGWDHTDTKRLLWVNGQFDPWLHATVSSPS | |
| | RPGGPLQSTDKAPVLVIPGGVHCTDLIIRNGDANEGARKVQSQAREIIKKWVSEFPKSGK | |
| | SP | |
| 5 | ATGGTGTCCCTCACGCATATATTTTCGAAGGCCCTCCTCACACTGCTGGTGGGCCAGTCT | Aspergillus |
| | GCTGCCCTAAGCTTTCTCCCCGGCATCAAGGCCAATAATCTCCAACTCGCCTCGGTATTA | flavus |
| | GGTATCGATGGCCATACCGCCAGGTTCAATCCTGAGAAGATCGCAGAGACCGCTATCTCG | Full- |
| | CGCGGTTCTGGCTCAGAAGTCCCTGCCCGGCGGATATCGGTATGTCTTTACCAGTCAAGC | length |
| | TTTCTAGTATATGAGGTAAAATCTAACTCGGCGTTCAGATCCCCATTGACCATGAGGATC | Af1Pro3 |
| | CATCTATGGGCACCTATCAGAACCGCTACTGGGTTTCAGCAGACTTTTACAAGCCCGGTG | gene |
| | GCCCCGTCTTTGTACTAGATGCCGGTGAAGGCAATGCCTACTCCGTGGCGCAATCGTATC | |
| | TCGGCGGATCGGATAACTTCTTCGCGGAGTACCTCAAGGAATTCAATGGGCTGGGCCTTG | |
| | TGTGGGAGCATCGGTGAGCCACCTACCCTAGTCATCATTGTCATGATTGACCGCTAACCT | |
| | CCGGTCCGATTGAAGTTACTATGGTGACTCTCTGCCCTTCCCTGTCAACACTAGCACCCC | |
| | CAACGAGCATTTCAAGTACCTCACCAACAGCCAGGCACTGGCTGACCTCCCTTACTTCGC | |
| | TGAGAAGTTCACTCTCAACGGGACAGACTTGAGCCCCAAGTCCAGTCCCTGGATCATGCT | |
| | CGGTGGCTCATACCCGGGCATGCGCGCGGCCTTCACCCGCAACGAGTACCCGGACACCAT | |
| | TTTCGCCTCGTTCGCCATGTCTGCGCCCGTCGAAGCCTGGGTCAACATGACCATCTACTT | |
| | CGAGCAAGTCTACCGCGGCATGGTTGCGAACGGACTGGGCGGCTGTGCCAAGGACCTCAA | |
| | GGCCATCAACGACTACATCGACAGCCAACTCGACAAGAAGGGCCAAGCCGCCGACGCCAT | |
| | CAAGACACTCTTCCTCGGTAAAGAAGGCATCCACAACTCCAACGGCGACTTCACCGCCGC | |
| | GCTCGGAAGCATCTACAACCTCTTCCAGAGCTACGGCGTCGACGGCGGCGAAGAAAGTCT | |
| | CTCCCAGCTCTGCAGCTACCTCGACAAAGAAGCCAGCCCCAACGGCATCGCCCGGAAAAT | |
| | CGGAGTCAAGGAACTGACCGAGAAGTTCGCCGCCTGGCCCCCGCTTCTGTACCTCATCAA | |
| | CCAGTGGGGCAGCCAGGTCGGTAACGGCGACTCCAACTGCAAGGGCCAGAACAATTCCAC | |
| | CGAGACCGTCTGTGAGCTGGGCGGGCAGTTCACCGACCCCGACACCATCAGCTGGACCTG | |
| | GCAGTACTGCACCGAATGGGGCTATCTCCAGGCCGACAACGTGGGCCCTCACTCCCTACT | |
| | CTCCAAGTACCAGTCCCTGGAGTACCAGCAGTCCCTTTGCTACCGACAGTTCCCCGGCGC | |
| | AAAGGAGAGTGGCCTGCTCCCCGAGCACCCGGAGGCGAACGAGACGAACGCCGAAACAGG | |
| | CGGATGGACCATCCGTCCTTCCAATGTCTTCTGGAGCGCGGGCGAGTTCGATCCCTGGCG | |
| | GACGTTGACGCCCTTGTCGAATGAGACATTCGCGCCGAAGGGCGTGCAGATCTCCACCAA | |
| | TATCCCCAAGTGTGGTGTCGAGACACCTGAGAATGTGCTCTTCGGCTATGTCATTCCGAG | |
| | GGCGGAGCATTGCTTTGACTATGACTTGAGTTACAAGCCGGCTGATAAGTCGCGGAAGTT | |
| | GTTCAGTCTTGCCTTGAAGAAGTGGCTCCCGTGCTGGCGGTCGGAGCATGCTCCTAAGGG | |
| | TGTACAGAGGAAGTGGATGTAA | |
| 6 | MVSLTHIFSKALLTLLVGQSAALSFLPGIKANNLQLASVLGIDGHTARFNPEKIAETAIS | Full- |
| | RGSGSEVPARRISIPIDHEDPSMGTYQNRYWVSADFYKPGGPVFVLDAGEGNAYSVAQSY | length |
| | LGGSDNEFAEYLKEENGLGLVWEHRYYGDSLPFPVNTSTPNEHFKYLTNSQALADLPYFA | AflPro3 |
| | EKFTLNGTDLSPKSSPWIMLGGSYPGMRAAFTRNEYPDTIFASFAMSAPVEAWVNMTIYF | precursor |
| | EQVYRGMVANGLGGCAKDLKAINDYIDSQLDKKGQAADAIKTLFLGKEGIHNSNGDFTAA | protein; |
| | LGSIYNLFQSYGVDGGEESLSQLCSYLDKEASPNGIARKIGVKELTEKFAAWPPLLYLIN | PRT; |
| | QWGSQVGNGDSNCKGQNNSTETVCELGGQFTDPDTISWTWQYCTEWGYLQADNVGPHSLL | Aspergillus |
| | SKYQSLEYQQSLCYRQFPGAKESGLLPEHPEANETNAETGGWTIRPSNVFWSAGEFDPWR | flavus |
| | TLTPLSNETFAPKGVQISTNIPKCGVETPENVLFGYVIPRAEHCFDYDLSYKPADKSRKL | |
| | FSLALKKWLPCWRSEHAPKGVQRKWM | |
| 7 | ATGAGGTTTCTCCAAAACCTACTCGGGGGCACTGCTTTGGCACTGCTTACAGGCCTTGGG | Full- |
| | TCGGCCTTTGGACCAAGATGGGCACGCTATCAGAACGACCTTCACCTAGCTGCAATGCTA | length |
| | GGTATGGATGCTGATTCTGTCTTGACCAACCGAAGCAGCCTCGCCTCTGCCATTGACAGT | CpoPro1 |
| | CTTGCCGAGACATCCGCTGTGGTCGCTGAATACGCAAATGTACGCCATCCCCATAGCTCC | gene; |
| | TTTGGGTGTGCTCTGTCTTAATTCCTGAAATAGATTCCTATCGATCACAGAAACCCTGGA | Coccidioides |
| | AGAATGTACAGGAATCGATACTGGGTGAACGATCAATATTATCAGCCAGGAGGGCCTGTG | posadasii |
| | GTTATTTTCGATACCGGTGAGACCAATGGTCAAGCTTTTGCCGATTATTATTTGGTCGAT | str.C735 |
| | CCTACGTCCTACATTGTCCAATTGCTTCGGGAATTTCATGGCGTAGGCCTTGTTTGGGAG | delta |
| | CACCGGTATGAAGTCAATTTCTACTAATAGGAACGGATAGGAGGCTAACTTTATGGAAGA | SOWgp |
| | TATTATGGCGAATCTCTCCCTTACCCCGTCAATGGGCAGACGTCTGCTGCGCAATTCCAA | |
| | TACTTGACGCTCGAACAAGCTTTGCAGGATCTCCCTTACTTTGCCAGAACATTTCGCCGA | |
| | CCTCGGCTCCCTAATGCTGATCTGACACCAAGATCAACCCCGTGGATTATGGTCGGCGGC | |
| | TCATACCCAGGCATGCGTGCAGCTTTCTCGAGACTCAAGTATCCCGACACTATTTTTGCT | |
| | GCCTTTTCCTCTTCTGCACCTGCTCAAGCTAGGATTGACATGAGCGTTTATTATGAGCAG | |
| | GTGTACAGGGGTTTGGTAGCATATGGCTATGGAAATTGCACCAGGGACGTCAATGCTGCA | |
| | TACCGATATATTGATGCCCAACTTGCCAACCCCAGTACCGCTGCTCAAATTAAGAGACAA | |
| | TTCTTAGGTCCCGGTGCCGAGCAAAACAGCAATGGCGATTTTACTGCAGTTTTGCTCTAT | |
| | AATTGGGCGACATGGCAGAGCTTTGGGGCAAATGGCCCTGCGGGTCAGTTCTGTAATTGG | |
| | CTCGAAACAGACCAATATGGCAGAGTGGCCCCTGCTGAAGGCTGGGCACCTTCAAGAGGT | |
| | GCTAGATCTGTGGTCGACAGATGGGCTGCATGGCCGGGACTCAGCCGAGCGATCAACTCC | |
| | ATTTTTGAAACAAACTGCAATTGCCCAGAAGAGACTTGCTCCTGTGACCTTTCTGCGCCA | |
| | CCTGCAGACCCCCTGGCCATCAGCTGGTCGTGGCAGTTTTGCTCGCAGTTCGGTTACTTC | |
| | CAGTACCAGAATCCCAGACCCCATGAGATCGCTTCGCGCTATCAAACGGAAGCTTACATC | |
| | CAAGATAACTGCTACCGGCAGTTCCCTGACGGCGTGAGCAGCGGCCATCTTCCCCGCCGC | |
| | CCTCGAGCCGATGCGACAAACAATTATACTGGAGGCTGGAACATGCGCCCTTCAAACGTC | |
| | TTCCACGGCGCTGGACAATACGACCCGTGGACTCCTTTGACTGTGCTTTCCCAGGAGCCT | |
| | TGGGGACCACGCCGTCGCGTCACCACTCAAATCCCGGCGTGCAATCAGGAACAAGAGGCG | |
| | GTTTTTGGCGTCCTGCTTCCCAATGCAGAGCACGTTTACGATCTTCAAACCTCTTACCAA | |
| | CCGGGCGAGGTATCCAGGCAACTGTTCAGAAGGGCGCTGCACCAGTGGCTTCCTTGCTTC | |
| | CGGAGGAGGAATTCAACGGCAGATCATGATTGA | |
| 8 | MRFLQNLLGGTALALLTGLGSAFGPRWARYQNDLHLAAMLGMDADSVLTNRSSLASAIDS | Full- |
| | LAETSAVVAEYANIPIDHRNPGRMYRNRYWVNDQYYQPGGPVVIFDTGETNGQAFADYYL | length |
| | VDPTSYIVQLLREFHGVGLVWEHRYYGESLPYPVNGQTSAAQFQYLTLEQALQDLPYFAR | CpoPro1 |
| | TFRRPRLPNADLTPRSTPWIMVGGSYPGMRAAFSRLKYPDTIFAAFSSSAPAQARIDMSV | precursor |
| | YYEQVYRGLVAYGYGNCTRDVNAAYRYIDAQLANPSTAAQIKRQFLGPGAEQNSNGDFTA | protein; |
| | VLLYNWATWQSFGANGPAGQFCNWLETDQYGRVAPAEGWAPSRGARSVVDRWAAWPGLSR | PRT; |
| | AINSIFETNCNCPEETCSCDLSAPPADPLAISWSWQFCSQFGYFQYQNPRPHEIASRYQT | Coccidioides |
| | EAYIQDNCYRQFPDGVSSGHLPRRPRADATNNYTGGWNMRPSNVFHGAGQYDPWTPLTVL | posadasii |
| | SQEPWGPRRRVTTQIPACNQEQEAVFGVLLPNAEHVYDLQTSYQPGEVSRQLFRRALHQW | str.C735 |
| | LPCFRRRNSTADHD | delta |
| | | SOWgp |
| 9 | VRVGNLLEPPMPPPFAIEDIEDIDPKQLTKRKISSGFFDQYIDHSNPSLGTFRQKFWWSD | MorPro1 |
| | EFYKGPGSPVILFNPGESRADIYTGYLTNLTVPGMYAQAVGAAVVMLEHRYWGESSPFAN | predicted |
| | LSTKNMQYLTLNNSISDTTRFARQVKLPFDTSGATNAPNAPWVFVGGSYPGALAGWVESV | mature |
| | APGTFWAYHASSAVVQDIGDYWRYFSPINEGMPKNCSADIGRVVEHIDKVLGTGSDSDKS | enzyme; |
| | ALQTAFGLGSLEHDDFVETLANGPYLWQGIDFSTGYSDFFKFCDYVENVPPKAATRVPPG | PRT; |
| | VDGVGLEKALTGYQDWIKKEYLPTACDSLGYPKGDLGCLSSHNFSAPFYRDQTVLNPGNR | Magnaporthe |
| | QWFWFLCNEPFKFWQNGAPKGEPSIVSRIIGSKYFESQCALWFPDEPREGGGVYTYGIAE | oryzae |
| | GKDVASVNKFTGGWDHTDTKRLLWVNGQFDPWLHATVSSPSRPGGPLQSTDKAPVLVIPG | 70-15 |
| | GVHCTDLIIRNGDANEGARKVQSQAREIIKKWVSEFPKSGKSP | |
| 10 | LSFLPGIKANNLQLASVLGIDGHTARFNPEKIAETAISRGSGSEVPARRISIPIDHEDPS | AflPro3 |
| | MGTYQNRYWVSADFYKPGGPVFVLDAGEGNAYSVAQSYLGGSDNFFAEYLKEFNGLGLVW | predicted, |
| | EHRYYGDSLPFPVNTSTPNEHFKYLTNSQALADLPYFAEKFTLNGTDLSPKSSPWIMLGG | mature; |
| | SYPGMRAAFTRNEYPDTIFASFAMSAPVEAWVNMTIYFEQVYRGMVANGLGGCAKDLKAI | PRT; |
| | NDYIDSQLDKKGQAADAIKTLFLGKEGIHNSNGDFTAALGSIYNLFQSYGVDGGEESLSQ | Aspergillus |
| | LCSYLDKEASPNGIARKIGVKELTEKFAAWPPLLYLINQWGSQVGNGDSNCKGQNNSTET | flavus |
| | VCELGGQFTDPDTISWTWQYCTEWGYLQADNVGPHSLLSKYQSLEYQQSLCYRQFPGAKE | |
| | SGLLPEHPEANETNAETGGWTIRPSNVFWSAGEFDPWRTLTPLSNETFAPKGVQISTNIP | |
| | KCGVETPENVLFGYVIPRAEHCFDYDLSYKPADKSRKLFSLALKKWLPCWRSEHAPKGVQ | |
| | RKWM | |
| 11 | FGPRWARYQNDLHLAAMLGMDADSVLTNRSSLASAIDSLAETSAVVAEYANIPIDHRNPG | CpoPro1 |
| | RMYRNRYWVNDQYYQPGGPVVIFDTGETNGQAFADYYLVDPTSYIVQLLREFHGVGLVWE | predicted, |
| | HRYYGESLPYPVNGQTSAAQFQYLTLEQALQDLPYFARTFRRPRLPNADLTPRSTPWIMV | mature |
| | GGSYPGMRAAFSRLKYPDTIFAAFSSSAPAQARIDMSVYYEQVYRGLVAYGYGNCTRDVN | enzyme; |
| | AAYRYIDAQLANPSTAAQIKRQFLGPGAEQNSNGDFTAVLLYNWATWQSFGANGPAGQFC | PRT; |
| | NWLETDQYGRVAPAEGWAPSRGARSVVDRWAAWPGLSRAINSIFETNCNCPEETCSCDLS | Coccidioides |
| | APPADPLAISWSWQFCSQFGYFQYQNPRPHEIASRYQTEAYIQDNCYRQFPDGVSSGHLP | posadasii |
| | RRPRADATNNYTGGWNMRPSNVFHGAGQYDPWTPLTVLSQEPWGPRRRVTTQIPACNQEQ | str.C735 |
| | EAVFGVLLPNAEHVYDLQTSYQPGEVSRQLFRRALHQWLPCFRRRNSTADHD | delta SOWgp |
| 12 | ATGCTCTTTCTGAGCTCCCTCCTGCTGCTCGCTCTCAGCGGCGCTCCCGCCTACGCCGTT | Synthesized |
| | CGAGTTGGCAACCTCCTGGAGCCTCCCATGCCTCCTCCCTTTGCTATTGAGGACATCGAA | nucleotide |
| | GACATTGACCCTAAGCAGCTCACCAAGCGAAAAATCAGCAGCGGTTTCTTCGACCAGTAC | sequence |
| | ATCGACCACTCCAACCCCAGCCTCGGTACTTTCCGCCAAAAGTTTTGGTGGTCCGACGAG | encoding |
| | TTCTACAAGGGCCCCGGTTCCCCCGTCATCCTGTTCAACCCTGGCGAAAGCCGCGCTGAT | full- |
| | ATCTACACCGGCTATCTGACTAACCTCACCGTCCCCGGCATGTACGCTCAAGCCGTCGGT | length |
| | GCTGCCGTTGTCATGCTGGAGCACCGCTATTGGGGCGAGTCCAGCCCCTTCGCCAATCTC | MorPro1 |
| | TCCACCAAGAACATGCAGTACCTGACCCTCAACAACAGCATTAGCGACACCACCCGCTTT | |
| | GCCCGCCAGGTCAAGCTGCCCTTTGACACCTCCGGCGCCACCAATGCTCCTAATGCCCCC | |
| | TGGGTCTTTGTCGGTGGTAGCTATCCTGGTGCCCTGGCCGGTTGGGTCGAGAGCGTTGCT | |
| | CCTGGCACCTTCTGGGCCTATCATGCCAGCTCCGCCGTCGTTCAAGATATCGGCGACTAT | |
| | TGGCGCTACTTTAGCCCCATCAACGAGGGCATGCCTAAAAACTGCAGCGCCGACATCGGT | |
| | CGCGTCGTCGAACACATCGATAAGGTCCTGGGTACCGGCTCCGACAGCGATAAGAGCGCC | |
| | CTGCAGACCGCTTTCGGCCTCGGCAGCCTGGAACACGACGACTTCGTCGAGACCCTCGCC | |
| | AACGGCCCCTACCTCTGGCAGGGCATCGACTTCAGCACTGGCTACAGCGACTTCTTCAAG | |
| | TTCTGCGACTACGTCGAGAATGTCCCTCCCAAGGCCGCCACTCGCGTTCCTCCCGGCGTC | |
| | GACGGCGTCGGCCTGGAGAAGGCCCTGACCGGTTACCAGGACTGGATCAAGAAGGAGTAC | |
| | CTCCCCACCGCCTGCGATTCCCTCGGCTACCCCAAAGGCGATCTCGGTTGCCTCAGCTCC | |
| | CACAACTTCTCCGCCCCTTTCTACCGCGATCAGACCGTCCTCAACCCCGGTAATCGCCAG | |
| | TGGTTCTGGTTCCTCTGCAACGAGCCCTTCAAGTTCTGGCAAAACGGCGCCCCCAAGGGC | |
| | GAGCCCAGCATCGTCAGCCGCATTATTGGCAGCAAGTACTTCGAGTCCCAGTGCGCCCTC | |
| | TGGTTTCCCGATGAGCCCCGCGAGGGCGGCGGTGTTTATACTTACGGCATCGCCGAAGGT | |
| | AAGGATGTCGCCAGCGTCAATAAGTTTACTGGCGGCTGGGACCATACTGACACCAAACGC | |
| | CTCCTGTGGGTTAACGGCCAGTTCGACCCCTGGCTCCACGCCACTGTCAGCAGCCCTAGC | |
| | CGACCCGGTGGCCCCCTCCAGAGCACTGACAAGGCCCCTGTCCTCGTTATTCCCGGCGGC | |
| | GTCCACTGCACCGATCTCATCATCCGCAACGGCGACGCTAACGAAGGCGCTCGCAAGGTT | |
| | CAAAGCCAGGCCCGCGAGATCATTAAGAAGTGGGTCAGCGAGTTTCCTAAAAGCGGCAAG | |
| | TCCCCCTAA | |
| 13 | ATGGTCTCCCTCACTCATATTTTCTCCAAGGCCCTCCTGACTCTCCTGGTCGGTCAATCC | Synthesized |
| | GCCGCTCTGAGCTTCCTCCCCGGTATCAAGGCTAACAATCTGCAACTGGCTTCCGTCCTG | nucleotide |
| | GGCATTGACGGCCACACCGCTCGCTTTAATCCCGAAAAAATCGCTGAAACCGCCATCTCC | sequence |
| | CGCGGTTCCGGCTCCGAGGTTCCCGCTCGACGCATCTCCATCCCCATCGATCATGAGGAC | encoding |
| | CCTTCCATGGGCACCTACCAGAACCGCTATTGGGTCTCCGCCGATTTCTACAAGCCCGGT | full- |
| | GGCCCCGTTTTCGTCCTCGATGCCGGTGAAGGCAACGCCTACTCCGTTGCCCAGTCCTAC | length |
| | CTCGGTGGCAGCGACAATTTCTTCGCCGAGTACCTGAAGGAGTTCAACGGTCTGGGCCTC | AflPro3 |
| | GTCTGGGAACACCGATACTACGGCGATTCCCTGCCCTTCCCCGTCAACACTTCCACCCCT | |
| | AACGAGCACTTCAAGTATCTCACCAACTCCCAGGCTCTCGCCGACCTCCCTTACTTTGCC | |
| | GAAAAGTTTACCCTGAACGGCACCGATCTGTCCCCCAAATCCAGCCCCTGGATTATGCTG | |
| | GGTGGTAGCTATCCCGGCATGCGAGCTGCTTTCACCCGCAATGAGTACCCCGATACCATT | |
| | TTCGCCAGCTTCGCCATGTCCGCTCCCGTTGAGGCCTGGGTCAACATGACTATCTACTTC | |
| | GAGCAAGTCTACCGCGGCATGGTTGCCAATGGCCTCGGCGGTTGCGCTAAGGATCTGAAA | |
| | GCCATCAACGATTATATCGACAGCCAACTGGACAAGAAAGGTCAAGCCGCCGACGCTATC | |
| | AAAACCCTCTTTCTCGGCAAGGAAGGCATCCACAACAGCAATGGCGACTTTACCGCCGCC | |
| | CTCGGTTCCATCTACAACCTGTTCCAAAGCTATGGCGTCGACGGTGGCGAGGAAAGCCTG | |
| | AGCCAGCTCTGCAGCTATCTCGACAAGGAGGCCAGCCCTAATGGCATCGCCCGCAAGATC | |
| | GGCGTCAAAGAGCTGACCGAGAAGTTCGCCGCTTGGCCCCCCCTGCTCTACCTCATCAAC | |
| | CAGTGGGGCTCCCAAGTTGGTAACGGCGACAGCAACTGTAAAGGCCAGAACAACTCCACC | |
| | GAAACTGTCTGCGAACTGGGCGGTCAGTTCACCGACCCCGACACCATTTCCTGGACCTGG | |
| | CAGTACTGCACTGAATGGGGCTACCTCCAGGCTGATAACGTCGGCCCTCACAGCCTCCTC | |
| | AGCAAGTACCAGAGCCTCGAATACCAGCAGTCCCTGTGCTACCGCCAATTCCCCGGCGCC | |
| | AAGGAGAGCGGTCTCCTGCCCGAGCACCCTGAGGCCAATGAGACCAACGCCGAGACTGGT | |
| | GGCTGGACCATCCGCCCTAGCAACGTCTTCTGGTCCGCCGGCGAATTTGATCCCTGGCGC | |
| | ACCCTCACCCCCCTCTCCAACGAGACCTTCGCTCCTAAGGGCGTCCAGATCTCCACCAAT | |
| | ATCCCCAAGTGCGGCGTTGAAACCCCTGAGAACGTCCTCTTCGGCTACGTCATCCCCCGA | |
| | GCCGAACACTGCTTCGACTACGACCTGTCCTACAAACCCGCCGACAAGAGCCGCAAACTG | |
| | TTCAGCCTCGCCCTGAAGAAGTGGCTGCCCTGTTGGCGCAGCGAGCACGCCCCTAAAGGC | |
| | GTTCAGCGCAAGTGGATGTAA | |
| 14 | ATGCGCTTTCTGCAAAATCTCCTGGGCGGCACTGCTCTGGCTCTCCTCACTGGCCTCGGC | Synthesized |
| | TCCGCCTTTGGTCCCCGCTGGGCCCGCTACCAAAACGATCTCCACCTGGCCGCTATGCTG | nucleotide |
| | GGCATGGACGCCGACAGCGTCCTGACCAACCGCAGCAGCCTCGCCTCCGCCATTGATTCC | sequence |
| | CTGGCTGAAACTTCCGCCGTCGTCGCCGAATACGCCAACATTCCCATCGACCACCGAAAC | encoding |
| | CCCGGTCGCATGTACCGCAACCGATACTGGGTCAACGACCAATATTACCAGCCCGGTGGC | full- |
| | CCTGTCGTTATCTTCGACACCGGCGAAACTAATGGCCAAGCCTTTGCTGACTACTACCTC | length |
| | GTCGACCCCACCTCCTATATCGTCCAACTCCTCCGCGAGTTCCATGGCGTCGGCCTCGTC | CpoPro1 |
| | TGGGAGCATCGCTACTACGGCGAGAGCCTCCCCTACCCCGTCAACGGCCAGACCTCCGCT | |
| | GCCCAATTCCAATATCTCACTCTGGAGCAGGCCCTCCAAGATCTGCCCTACTTCGCCCGA | |
| | ACTTTCCGACGACCCCGCCTGCCTAATGCCGATCTCACCCCCCGAAGCACCCCCTGGATC | |
| | ATGGTCGGCGGTTCCTATCCTGGCATGCGCGCTGCTTTTAGCCGACTGAAGTACCCCGAC | |
| | ACTATTTTTGCCGCCTTCAGCAGCTCCGCTCCCGCTCAGGCCCGCATTGACATGAGCGTC | |
| | TACTACGAGCAGGTTTATCGCGGCCTGGTCGCTTATGGTTACGGCAACTGCACTCGCGAC | |
| | GTTAATGCTGCCTACCGCTACATTGACGCCCAGCTCGCCAACCCTAGCACTGCCGCTCAA | |
| | ATCAAACGCCAATTTCTCGGTCCCGGTGCCGAGCAGAATAGCAACGGCGACTTCACTGCT | |
| | GTCCTGCTCTACAACTGGGCCACTTGGCAATCCTTTGGCGCTAATGGTCCTGCCGGCCAG | |
| | TTTTGTAACTGGCTGGAGACCGACCAGTACGGTCGAGTCGCCCCTGCCGAAGGCTGGGCT | |
| | CCTTCCCGCGGTGCTCGATCCGTTGTCGACCGATGGGCTGCCTGGCCCGGTCTGTCCCGC | |
| | GCTATTAACTCCATTTTTGAGACTAATTGTAATTGTCCCGAAGAGACCTGTAGCTGCGAC | |
| | CTCAGCGCCCCTCCTGCTGACCCTCTGGCCATCAGCTGGAGCTGGCAGTTCTGCAGCCAA | |
| | TTCGGCTACTTCCAGTACCAGAATCCTCGCCCCCACGAGATCGCTAGCCGATACCAGACT | |
| | GAGGCTTATATCCAAGACAATTGCTACCGACAGTTCCCCGACGGCGTTAGCTCCGGTCAC | |
| | CTGCCCCGCCGCCCTCGAGCCGATGCCACTAACAACTACACTGGCGGCTGGAACATGCGC | |
| | CCCAGCAATGTCTTTCACGGCGCTGGTCAGTATGACCCTTGGACTCCCCTCACCGTCCTG | |
| | TCCCAGGAACCTTGGGGCCCTCGCCGCCGAGTCACCACTCAGATCCCCGCCTGCAATCAA | |
| | GAACAGGAGGCCGTCTTCGGTGTTCTCCTCCCCAACGCCGAACACGTTTACGACCTGCAG | |
| | ACCAGCTATCAACCTGGTGAGGTCAGCCGACAACTGTTTCGACGCGCCCTGCATCAGTGG | |
| | CTGCCCTGCTTTCGACGCCGCAACTCCACCGCTGATCATGACTAA | |
| 15 | MAKLSTLRLASLLSLVSVQVSASVHLLESLEKLPHGWKAAETPSPSSQIVLQVALTQQNI | Trichoderma |
| | DQLESRLAAVSTPTSSTYGKYLDVDEINSIFAPSDASSSAVESWLQSHGVTSYTKQGSSI | reesei |
| | WFQTNISTANAMLSTNEHTYSDLTGAKKVRTLKYSIPESLIGHVDLISPTTYFGTTKAMR | QM6a |
| | KLKSSGVSPAADALAARQEPSSCKGTLVFEGETFNVFQPDCLRTEYSVDGYTPSVKSGSR | (1) |
| | IGEGSFLNESASFADQALFEKHENIPSQNFSVVLINGGTDLPQPPSDANDGEANLDAQTI | Leader |
| | LTIAHPLPITEFITAGSPPYFPDPVEPAGTPNENEPYLQYYEFLLSKSNAEIPQVITNSY | |
| | GDEEQTVPRSYAVRVCNLIGLLGLRGISVLHSSGDEGVGASCVATNSTTPQFNPIFPATC | |
| | PYVTSVGGTVSFNPEVAWAGSSGGFSYYFSRPWYQQEAVGTYLEKYVSAETKKYYGPYVD | |
| | FSGRGFPDVAAHSVSPDYPVFQGGELTPSGGTSAASPVVAAIVALLNDARLREGKPTLGF | |
| | LNPLIYLHASKGFTDITSGQSEGCNGNNTQTGSPLPGAGFIAGAHWNATKGWDPTTGFGV | |
| | PNLKKLLALVRF | |
| 16 | SVHLLESLEKLPHGWKAAETPSPSSQIVLQVALTQQNIDQLESRLAAVSTPTSSTYGKYL | Trichoderma |
| | DVDEINSIFAPSDASSSAVESWLQSHGVTSYTKQGSSIWFQTNISTANAMLSTNEHTYSD | reesei |
| | LTGAKKVRTLKYSIPESLIGHVDLISPTTYFGTTKAMRKLKSSGVSPAADALAARQEPSS | QM6a |
| | CKGTLVFEGETFNVFQPDCLRTEYSVDGYTPSVKSGSRIGFGSFLNESASFADQALFEKH | (2) |
| | FNIPSQNFSVVLINGGTDLPQPPSDANDGEANLDAQTILTIAHPLPITEFITAGSPPYFP | |
| | DPVEPAGTPNENEPYLQYYEFLLSKSNAEIPQVITNSYGDEEQTVPRSYAVRVCNLIGLL | |
| | GLRGISVLHSSGDEGVGASCVATNSTTPQFNPIFPATCPYVTSVGGTVSFNPEVAWAGSS | |
| | GGFSYYFSRPWYQQEAVGTYLEKYVSAETKKYYGPYVDFSGRGFPDVAAHSVSPDYPVFQ | |
| | GGELTPSGGTSAASPVVAAIVALLNDARLREGKPTLGELNPLIYLHASKGFTDITSGQSE | |
| | GCNGNNTQTGSPLPGAGFIAGAHWNATKGWDPTTGEGVPNLKKLLALVRF | |
| 17 | EAFEKLSAVPKGWHYSSTPKGNTEVCLKIALAQKDAAGFEKTVLEMSDPDHPSYGQHFTT | Aspergillus |
| | HDEMKRMLLPRDDTVDAVRQWLENGGVTDFTQDADWINFCTTVDTANKLLNAQFKWYVSD | oryzae |
| | VKHIRRLRTLQYDVPESVTPHINTIQPTTREGKISPKKAVTHSKPSQLDVTALAAAVVAK | RIB40 |
| | NISHCDSIITPTCLKELYNIGDYQADANSGSKIAFASYLEEYARYADLENFENYLAPWAK | (3) |
| | GQNFSVTTENGGLNDQNSSSDSGEANLDLQYILGVSAPLPVTEFSTGGRGPLVPDLTQPD | |
| | PNSNSNEPYLEFFQNVLKLDQKDLPQVISTSYGENEQEIPEKYARTVCNLIAQLGSRGVS | |
| | VLFSSGDSGVGEGCMTNDGTNRTHFPPQFPAACPWVTSVGATFKTTPERGTYFSSGGFSD | |
| | YWPRPEWQDEAVSSYLETIGDTFKGLYNSSGRAFPDVAAQGMNFAVYDKGTLGEFDGTSA | |
| | SAPAFSAVIALLNDARLRAGKPTLGELNPWLYKTGRQGLQDITLGASIGCTGRAREGGAP | |
| | DGGPVVPYASWNATQGWDPVTGLGTPDFAELKKLALGN | |
| 18 | EPFEKLFSTPEGWKMQGLATNEQIVKLQIALQQGDVAGFEQHVIDISTPSHPSYGAHYGS | Phaeosphaeria |
| | HEEMKRMIQPSSETVASVSAWLKAAGINDAEIDSDWVTEKTTVGVANKMLDTKFAWYVSE | nodorum |
| | EAKPRKVLRTLEYSVPDDVAEHINLIQPTTRFAAIRQNHEVAHEIVGLQFAALANNTVNC | SN15 |
| | DATITPQCLKTLYKIDYKADPKSGSKVAFASYLEQYARYNDLALFEKAFLPEAVGQNFSV | (4) |
| | VQFSGGLNDQNTTQDSGEANLDLQYIVGVSAPLPVTEFSTGGRGPWVADLDQPDEADSAN | |
| | EPYLEFLQGVLKLPQSELPQVISTSYGENEQSVPKSYALSVCNLFAQLGSRGVSVIFSSG | |
| | DSGPGSACQSNDGKNTTKFQPQYPAACPFVTSVGSTRYLNETATGFSSGGFSDYWKRPSY | |
| | QDDAVKAYFHHLGEKFKPYFNRHGRGFPDVATQGYGFRVYDQGKLKGLQGTSASAPAFAG | |
| | VIGLLNDARLKAKKPTLGFLNPLLYSNSDALNDIVLGGSKGCDGHARFNGPPNGSPVIPY | |
| | AGWNATAGWDPVTGLGTPNFPKLLKAAVPSRYRA | |
| 19 | NAAVLLDSLDKVPVGWQAASAPAPSSKITLQVALTQQNIDQLESKLAAVSTPNSSNYGKY | Trichoderma |
| | LDVDEINQIFAPSSASTAAVESWLKSYGVDYKVQGSSIWFQTDVSTANKMLSTNFHTYTD | atroviride |
| | SVGAKKVRTLQYSVPETLADHIDLISPTTYFGTSKAMRALKIQNAASAVSPLAARQEPSS | IMI206040 |
| | CKGTIEFENRTFNVFQPDCLRTEYSVNGYKPSAKSGSRIGFGSFLNQSASSSDLALFEKH | (5) |
| | FGFASQGFSVELINGGSNPQPPTDANDGEANLDAQNIVSFVQPLPITEFIAGGTAPYFPD | |
| | PVEPAGTPDENEPYLEYYEYLLSKSNKELPQVITNSYGDEEQTVPQAYAVRVCNLIGLMG | |
| | LRGISILESSGDEGVGASCLATNSTTTPQFNPIFPATCPYVTSVGGTVSFNPEVAWDGSS | |
| | GGFSYYFSRPWYQEAAVGTYLNKYVSEETKEYYKSYVDFSGRGFPDVAAHSVSPDYPVFQ | |
| | GGELTPSGGTSAASPIVASVIALLNDARLRAGKPALGFLNPLIYGYAYKGFTDITSGQAV | |
| | GCNGNNTQTGGPLPGAGVIPGAFWNATKGWDPTTGFGVPNFKKLLELVRY | |
| 20 | KPTPGASHKVIEHLDFVPEGWQMVGAADPAAIIDFWLAIERENPEKLYDTIYDVSTPGRA | Arthroderma |
| | QYGKHLKREELDDLLRPRAETSESIINWLTNGGVNPQHIRDEGDWVRFSTNVKTAETLMN | benhamiae |
| | TRFNVFKDNLNSVSKIRTLEYSVPVAISAHVQMIQPTTLFGRQKPQNSLILNPLTKDLES | CBS |
| | MSVEEFAASQCRSLVTTACLRELYGLGDRVTQARDDNRIGVSGFLEEYAQYRDLELFLSR | 112371 |
| | FEPSAKGFNFSEGLIAGGKNTQGGPGSSTEANLDMQYVVGLSHKAKVTYYSTAGRGPLIP | (6) |
| | DLSQPSQASNNNEPYLEQLRYLVKLPKNQLPSVLTTSYGDTEQSLPASYTKATCDLFAQL | |
| | GTMGVSVIFSSGDTGPGSSCQTNDGKNATRFNPIYPASCPFVTSIGGTVGTGPERAVSFS | |
| | SGGFSDRFPRPQYQDNAVKDYLKILGNQWSGLFDPNGRAFPDIAAQGSNYAVYDKGRMTG | |
| | VSGTSASAPAMAAIIAQLNDFRLAKGSPVLGFLNPWIYSKGFSGFTDIVDGGSRGCTGYD | |
| | IYSGLKAKKVPYASWNATKGWDPVTGFGTPNFQALTKVLP | |
| 21 | KSYSHHAEAPKGWKVDDTARVASTGKQQVFSIALTMQNVDQLESKLLDLSSPDSKNYGQW | Fusarium |
| | MSQKDVTTAFYPSKEAVSSVTKWLKSKGVKHYNVNGGFIDFALDVKGANALLDSDYQYYT | graminearum |
| | KEGQTKLRTLSYSIPDDVAEHVQFVDPSTNFGGTLAFAPVTHPSRTLTERKNKPTKSTVD | PH-1 |
| | ASCQTSITPSCLKQMYNIGDYTPKVESGSTIGFSSFLGESAIYSDVFLFEEKFGIPTQNF | (7) |
| | TTVLINNGTDDQNTAHKNFGEADLDAENIVGIAHPLPFTQYITGGSPPFLPNIDQPTAAD | |
| | NQNEPYVPFFRYLLSQKEVPAVVSTSYGDEEDSVPREYATMTCNLIGLLGLRGISVIFSS | |
| | GDIGVGAGCLGPDHKTVEFNAIFPATCPYLTSVGGTVDVTPEIAWEGSSGGFSKYFPRPS | |
| | YQDKAVKTYMKTVSKQTKKYYGPYTNWEGRGFPDVAGHSVSPNYEVIYAGKQSASGGTSA | |
| | AAPVWAAIVGLLNDARFRAGKPSLGWLNPLVYKYGPKVLTDITGGYAIGCDGNNTQSGKP | |
| | EPAGSGIVPGARWNATAGWDPVTGYGTPDFGKLKDLVLSF | |
| 22 | AVVIRAAVLPDAVKLMGKAMPDDIISLQFSLKQQNIDQLETRLRAVSDPSSPEYGQYMSE | Acremonium |
| | SEVNEFFKPRDDSFAEVIDWVAASGFQDIHLTPQAAAINLAATVETADQLLGANFSWFDV | alcalophilum |
| | DGTRKLRTLEYTIPDRLADHVDLISPTTYFGRARLDGPRETPTRLDKRQRDPVADKAYFH | (8) |
| | LKWDRGTSNCDLVITPPCLEAAYNYKNYMPDPNSGSRVSFTSFLEQAAQQSDLTKFLSLT | |
| | GLDRLRPPSSKPASFDTVLINGGETHQGTPPNKTSEANLDVQWLAAVIKARLPITQWITG | |
| | GRPPFVPNLRLRHEKDNTNEPYLEFFEYLVRLPARDLPQVISNSYAEDEQTVPEAYARRV | |
| | CNLIGIMGLRGVTVLTASGDSGVGAPCRANDGSDRLEFSPQFPTSCPYITAVGGTEGWDP | |
| | EVAWEASSGGFSHYFLRPWYQANAVEKYLDEELDPATRAYYDGNGFVQFAGRAYPDLSAH | |
| | SSSPRYAYIDKLAPGLTGGTSASCPVVAGIVGLLNDARLRRGLPTMGFINPWLYTRGFEA | |
| | LQDVTGGRASGCQGIDLQRGTRVPGAGIIPWASWNATPGWDPATGLGLPDFWAMRGLALG | |
| | RGT | |
| 23 | AVVIRAAPLPESVKLVRKAAAEDGINLQLSLKRQNMDQLEKFLRAVSDPFSPKYGQYMSD | Sodiomyces |
| | AEVHEIFRPTEDSFDQVIDWLTKSGFGNLHITPQAAAINVATTVETADQLFGANFSWFDV | alkalinus |
| | DGTPKLRTGEYTIPDRLVEHVDLVSPTTYFGRMRPPPRGDGVNDWITENSPEQPAPLNKR | (9) |
| | DTKTESDQARDHPSWDSRTPDCATIITPPCLETAYNYKGYIPDPKSGSRVSFTSFLEQAA | |
| | QQADLTKFLSLTRLEGFRTPASKKKTFKTVLINGGESHEGVHKKSKTSEANLDVQWLAAV | |
| | TQTKLPITQWITGGRPPFVPNLRIPTPEANTNEPYLEFLEYLFRLPDKDLPQVISNSYAE | |
| | DEQSVPEAYARRVCGLLGIMGLRGVTVLTASGDSGVGAPCRANDGSGREEFSPQFPSSCP | |
| | YITTVGGTQAWDPEVAWKGSSGGFSNYFPRPWYQVAAVEKYLEEQLDPAAREYYEENGFV | |
| | RFAGRAFPDLSAHSSSPKYAYVDKRVPGLTGGTSASCPVVAGIVGLLNDARLRRGLPTMG | |
| | FINPWLYAKGYQALEDVTGGAAVGCQGIDIQTGKRVPGAGIIPGASWNATPDWDPATGLG | |
| | LPNFWAMRELALED | |
| 24 | VVHEKLAAVPSGWHHLEDAGSDHQISLSIALARKNLDQLESKLKDLSTPGESQYGQWLDQ | Aspergillus |
| | EEVDTLFPVASDKAVISWLRSANITHIARQGSLVNFATTVDKVNKLLNTTFAYYQRGSSQ | kawachii |
| | RLRTTEYSIPDDLVDSIDLISPTTFFGKEKTSAGLTQRSQKVDNHVAKRSNSSSCADTIT | IFO 4308 |
| | LSCLKEMYNFGNYTPSASSGSKLGFASFLNESASYSDLAKFERLFNLPSQNFSVELINGG | (10) |
| | VNDQNQSTASLTEADLDVELLVGVGHPLPVTEFITSGEPPFIPDPDEPSAADNENEPYLQ | |
| | YYEYLLSKPNSALPQVISNSYGDDEQTVPEYYAKRVCNLIGLVGLRGISVLESSGDEGIG | |
| | SGCRTTDGTNSTQFNPIFPATCPYVTAVGGTMSYAPEIAWEASSGGFSNYFERAWFQKEA | |
| | VQNYLANHITNETKQYYSQFANFSGRGFPDVSAHSFEPSYEVIFYGARYGSGGTSAACPL | |
| | FSALVGMLNDARLRAGKSTLGELNPLLYSKGYKALTDVTAGQSIGCNGIDPQSDEAVAGA | |
| | GIIPWAHWNATVGWDPVTGLGLPDFEKLRQLVLSL | |
| 25 | AAALVGHESLAALPVGWDKVSTPAAGTNIQLSVALALQNIEQLEDHLKSVSTPGSASYGQ | Talaromyces |
| | YLDSDGIAAQYGPSDASVEAVTNWLKEAGVTDIYNNGQSIHFATSVSKANSLLGADFNYY | stipitatus |
| | SDGSATKLRTLAYSVPSDLKEAIDLVSPTTYFGKTTASRSIQAYKNKRASTTSKSGSSSV | ATCC |
| | QVSASCQTSITPACLKQMYNVGNYTPSVAHGSRVGEGSFLNQSAIFDDLFTYEKVNDIPS | 10500 |
| | QNFTKVIIANASNSQDASDGNYGEANLDVQNIVGISHPLPVTEFLTGGSPPFVASLDTPT | (11) |
| | NQNEPYIPYYEYLLSQKNEDLPQVISNSYGDDEQSVPYKYAIRACNLIGLTGLRGISVLE | |
| | SSGDLGVGAGCRSNDGKNKTQFDPIFPATCPYVTSVGGTQSVTPEIAWVASSGGFSNYFP | |
| | RTWYQEPAIQTYLGLLDDETKTYYSQYTNFEGRGFPDVSAHSLTPDYQVVGGGYLQPSGG | |
| | TSAASPVFAGIIALLNDARLAAGKPTLGFLNPFFYLYGYKGLNDITGGQSVGCNGINGQT | |
| | GAPVPGGGIVPGAAWNSTTGWDPATGLGTPDFQKLKELVLSF | |
| 26 | KSFSHHAEAPQGWQVQKTAKVASNTQHVFSLALTMQNVDQLESKLLDLSSPDSANYGNWL | Fusarium |
| | SHDELTSTFSPSKEAVASVTKWLKSKGIKHYKVNGAFIDFAADVEKANTLLGGDYQYYTK | oxysporum |
| | DGQTKLRTLSYSIPDDVAGHVQFVDPSTNEGGTVAFNPVPHPSRTLQERKVSPSKSTVDA | f. sp. |
| | SCQTSITPSCLKQMYNIGDYTPDAKSGSEIGFSSFLGQAAIYSDVFKFEELFGIPKQNYT | Cubense |
| | TILINNGTDDQNTAHGNFGEANLDAENIVGIAHPLPFKQYITGGSPPFVPNIDQPTEKDN | Race 4 |
| | QNEPYVPFFRYLLGQKDLPAVISTSYGDEEDSVPREYATLTCNMIGLLGLRGISVIFSSG | (12) |
| | DIGVGSGCLAPDYKTVEFNAIFPATCPYLTSVGGTVDVTPEIAWEGSSGGFSKYFPRPSY | |
| | QDKAIKKYMKTVSKETKKYYGPYTNWEGRGFPDVAGHSVAPDYEVIYNGKQARSGGTSAA | |
| | APVWAAIVGLLNDARFKAGKKSLGWLNPLIYKHGPKVLTDITGGYAIGCDGNNTQSGKPE | |
| | RAGSGLVPGARWNATAGWDPTTGYGTPNFQKLKDLVLSL | |
| 27 | SVLVESLEKLPHGWKAASAPSPSSQITLQVALTQQNIDQLESRLAAVSTPNSKTYGNYLD | Trichoderma |
| | LDEINEIFAPSDASSAAVESWLHSHGVTKYTKQGSSIWFQTEVSTANAMLSTNFHTYSDA | virens |
| | AGVKKLRTLQYSIPESLVGHVDLISPTTYFGTSNAMRALRSKSVASVAQSVAARQEPSSC | Gv29-8 |
| | KGTLVFEGRTFNVFQPDCLRTEYNVNGYTPSAKSGSRIGFGSFLNQSASFSDLALFEKHF | (13) |
| | GFSSQNFSVVLINGGTDLPQPPSDDNDGEANLDVQNILTIAHPLPITEFITAGSPPYFPD | |
| | PVEPAGTPDENEPYLQYFEYLLSKPNRDLPQVITNSYGDEEQTVPQAYAVRVCNLIGLMG | |
| | LRGISILESSGDEGVGASCVATNSTTPQFNPIFPATCPYVTSVGGTVNFNPEVAWDGSSG | |
| | GFSYYFSRPWYQEEAVGNYLEKHVSAETKKYYGPYVDFSGRGFPDVAAHSVSPDYPVFQG | |
| | GQLTPSGGTSAASPVVASIIALLNDARLREGKPTLGFLNPLIYQYAYKGFTDITSGQSDG | |
| | CNGNNTQTDAPLPGAGVVLGAHWNATKGWDPTTGFGVPNFKKLLELIRYI | |
| 28 | AVLVESLKQVPNGWNAVSTPDPSTSIVLQIALAQQNIDELEWRLAAVSTPNSGNYGKYLD | Trichoderma |
| | IGEIEGIFAPSNASYKAVASWLQSHGVKNFVKQAGSIWFYTTVSTANKMLSTDFKHYSDP | atroviride |
| | VGIEKLRTLQYSIPEELVGHVDLISPTTYFGNNHPATARTPNMKAINVTYQIFHPDCLKT | IMI |
| | KYGVDGYAPSPRCGSRIGFGSFLNETASYSDLAQFEKYFDLPNQNLSTLLINGAIDVQPP | 206040 |
| | SNKNDSEANMDVQTILTFVQPLPITEFVVAGIPPYIPDAALPIGDPVQNEPWLEYFEFLM | (14) |
| | SRTNAELPQVIANSYGDEEQTVPQAYAVRVCNQIGLLGLRGISVIASSGDTGVGMSCMAS | |
| | NSTTPQFNPMFPASCPYITTVGGTQHLDNEIAWELSSGGFSNYFTRPWYQEDAAKTYLER | |
| | HVSTETKAYYERYANFLGRGFPDVAALSLNPDYPVIIGGELGPNGGTSAAAPVVASIIAL | |
| | LNDARLCLGKPALGFLNPLIYQYADKGGFTDITSGQSWGCAGNTTQTGPPPPGAGVIPGA | |
| | HWNATKGWDPVTGFGTPNFKKLLSLALSV | |
| 29 | SPLARRWDDFAEKHAWVEVPRGWEMVSEAPSDHTFDLRIGVKSSGMEQLIENLMQTSDPT | Agaricus |
| | HSRYGQHLSKEELHDFVQPHPDSTGAVEAWLEDFGISDDFIDRTGSGNWVTVRVSVAQAE | bisporus |
| | RMLGTKYNVYRHSESGESVVRTMSYSLPSELHSHIDVVAPTTYFGTMKSMRVTSFLQPEI | var. |
| | EPVDPSAKPSAAPASCLSTTVITPDCLRDLYNTADYVPSATSRNAIGIAGYLDRSNRADL | burnetti |
| | QTFFRRFRPDAVGFNYTTVQLNGGGDDQNDPGVEANLDIQYAAGIAFPTPATYWSTGGSP | JB137-S8 |
| | PFIPDTQTPTNTNEPYLDWINFVLGQDEIPQVISTSYGDDEQTVPEDYATSVCNLFAQLG | (15) |
| | SRGVTVFFSSGDFGVGGGDCLTNDGSNQVLFQPAFPASCPFVTAVGGTVRLDPEIAVSFS | |
| | GGGFSRYFSRPSYQNQTVAQFVSNLGNTFNGLYNKNGRAYPDLAAQGNGFQVVIDGIVRS | |
| | VGGTSASSPTVAGIFALLNDFKLSRGQSTLGFINPLIYSSATSGFNDIRAGTNPGCGTRG | |
| | FTAGTGWDPVTGLGTPDFLRLQGLI | |
| 30 | RVFDSLPHPPRGWSYSHAAESTEPLTLRIALRQQNAAALEQVVLQVSNPRHANYGQHLTR | Magnaporthe |
| | DELRSYTAPTPRAVRSVTSWLVDNGVDDYTVEHDWVTLRTTVGAADRLLGADFAWYAGPG | oryzae |
| | ETLQLRTLSYGVDDSVAPHVDLVQPTTRFGGPVGQASHIFKQDDFDEQQLKTLSVGFQVM | 70-15 |
| | ADLPANGPGSIKAACNESGVTPLCLRTLYRVNYKPATTGNLVAFASFLEQYARYSDQQAF | (16) |
| | TQRVLGPGVPLQNFSVETVNGGANDQQSKLDSGEANLDLQYVMAMSHPIPILEYSTGGRG | |
| | PLVPTLDQPNANNSSNEPYLEFLTYLLAQPDSAIPQTLSVSYGEEEQSVPRDYAIKVCNM | |
| | FMQLGARGVSVMFSSGDSGPGNDCVRASDNATFFGSTFPAGCPYVTSVGSTVGFEPERAV | |
| | SFSSGGFSIYHARPDYQNEVVPKYIESIKASGYEKFFDGNGRGIPDVAAQGARFVVIDKG | |
| | RVSLISGTSASSPAFAGMVALVNAARKSKDMPALGFLNPMLYQNAAAMTDIVNGAGIGCR | |
| | KQRTEFPNGARFNATAGWDPVTGLGTPLFDKLLAVGAPGVPNA | |
| 31 | SDVVLESLREVPQGWKRLRDADPEQSIKLRIALEQPNLDLFEQTLYDISSPDHPKYGQHL | Togninia |
| | KSHELRDIMAPREESTAAVIAWLQDAGLSGSQIEDDSDWINIQTTVAQANDMLNTTFGLF | minima |
| | AQEGTEVNRIRALAYSVPEEIVPHVKMIAPIIRFGQLRPQMSHIFSHEKVEETPSIGTIK | UCRPA7 |
| | AAAIPSVDLNVTACNASITPECLRALYNVGDYEADPSKKSLFGVCGYLEQYAKHDQLAKF | (17) |
| | EQTYAPYAIGADFSVVTINGGGDNQTSTIDDGEANLDMQYAVSMAYKTPITYYSTGGRGP | |
| | LVPDLDQPDPNDVSNEPYLDFVSYLLKLPDSKLPQTITTSYGEDEQSVPRSYVEKVCTMF | |
| | GALGARGVSVIESSGDTGVGSACQTNDGKNTTRFLPIFPAACPYVTSVGGTRYVDPEVAV | |
| | SFSSGGFSDIFPTPLYQKGAVSGYLKILGDRWKGLYNPHGRGFPDVSGQSVRYHVFDYGK | |
| | DVMYSGTSASAPMFAALVSLLNNARLAKKLPPMGFLNPWLYTVGFNGLTDIVHGGSTGCT | |
| | GTDVYSGLPTPFVPYASWNATVGWDPVTGLGTPLFDKLLNLSTPNFHLPHIGGH | |
| 32 | STTSHVEGEVVERLHGVPEGWSQVGAPNPDQKLRFRIAVRSADSELFERTLMEVSSPSHP | Bipolaris |
| | RYGQHLKRHELKDLIKPRAKSTSNILNWLQESGIEARDIQNDGEWISFYAPVKRAEQMMS | maydiC5 |
| | TTFKTYQNEARANIKKIRSLDYSVPKHIRDDIDIIQPTTRFGQIQPERSQVFSQEEVPFS | (18) |
| | ALVVNATCNKKITPDCLANLYNFKDYDASDANVTIGVSGFLEQYARFDDLKQFISTFQPK | |
| | AAGSTFQVTSVNAGPFDQNSTASSVEANLDIQYTTGLVAPDIETRYFTVPGRGILIPDLD | |
| | QPTESDNANEPYLDYFTYLNNLEDEELPDVLTTSYGESEQSVPAEYAKKVCNLIGQLGAR | |
| | GVSVIESSGDTGPGSACQTNDGKNTTRFLPIFPASCPYVTSVGGTVGVEPEKAVSFSSGG | |
| | FSDLWPRPAYQEKAVSEYLEKLGDRWNGLYNPQGRGFPDVAAQGQGFQVFDKGRLISVGG | |
| | TSASAPVFASVVALLNNARKAAGMSSLGFLNPWIYEQGYKGLTDIVAGGSTGCTGRSIYS | |
| | GLPAPLVPYASWNATEGWDPVTGYGTPDFKQLLTLATAPKSGERRVRRGGLGGQA | |
| 33 | MLSSFLSQGAAVSLALLSLLPSPVAAEIFEKLSGVPNGWRYANNPHGNEVIRLQIALQQH | Aspergillus |
| | DVAGFEQAVMDMSTPGHADYGKHFRTHDEMKRMLLPSDTAVDSVRDWLESAGVHNIQVDA | kawachii |
| | DWVKFHTTVNKANALLDADFKWYVSEAKHIRRLRTLQYSIPDALVSHINMIQPTTRFGQI | IFO 4308 |
| | QPNRATMRSKPKHADETFLTAATLAQNTSHCDSIITPHCLKQLYNIGDYQADPKSGSKVG | (19) |
| | FASYLEEYARYADLERFEQHLAPNAIGQNFSVVQFNGGLNDQLSLSDSGEANLDLQYILG | |
| | VSAPVPVTEYSTGGRGELVPDLSSPDPNDNSNEPYLDFLQGILKLDNSDLPQVISTSYGE | |
| | DEQTIPVPYARTVCNLYAQLGSRGVSVIFSSGDSGVGAACLTNDGTNRTHFPPQFPASCP | |
| | WVTSVGATSKTSPEQAVSFSSGGESDLWPRPSYQQAAVQTYLTQHLGNKFSGLENASGRA | |
| | FPDVAAQGVNYAVYDKGMLGQFDGTSCSAPTFSGVIALLNDARLRAGLPVMGFLNPFLYG | |
| | VGSESGALNDIVNGGSLGCDGRNRFGGTPNGSPVVPFASWNATTGWDPVSGLGTPDFAKL | |
| | RGVALGEAKAYGN | |
| 34 | MAATGRFTAFWNVASVPALIGILPLAGSHLRAVLCPVCIWRHSKAVCAPDTLQAMRAFTR | Aspergillus |
| | VTAISLAGFSCFAAAAAAAFESLRAVPDGWIYESTPDPNQPLRLRIALKQHNVAGFEQAL | nidulans |
| | LDMSTPGHSSYGQHFGSYHEMKQLLLPTEEASSSVRDWLSAAGVEFEQDADWINFRTTVD | FGSC A4 |
| | QANALLDADFLWYTTTGSTGNPTRILRTLSYSVPSELAGYVNMIQPTTRFGGTHANRATV | (20) |
| | RAKPIFLETNRQLINAISSGSLEHCEKAITPSCLADLYNTEGYKASNRSGSKVAFASFLE | |
| | EYARYDDLAEFEETYAPYAIGQNFSVISINGGLNDQDSTADSGEANLDLQYIIGVSSPLP | |
| | VTEFTTGGRGKLIPDLSSPDPNDNTNEPFLDFLEAVLKLDQKDLPQVISTSYGEDEQTIP | |
| | EPYARSVCNLYAQLGSRGVSVLFSSGDSGVGAACQTNDGKNTTHFPPQFPASCPWVTAVG | |
| | GTNGTAPESGVYFSSGGFSDYWARPAYQNAAVESYLRKLGSTQAQYFNRSGRAFPDVAAQ | |
| | AQNFAVVDKGRVGLEDGTSCSSPVFAGIVALLNDVRLKAGLPVLGELNPWLYQDGLNGLN | |
| | DIVDGGSTGCDGNNRFNGSPNGSPVIPYAGWNATEGWDPVTGLGTPDFAKLKALVLDA | |
| 35 | MLSFVRRGALSLALVSLLTSSVAAEVFEKLHVVPEGWRYASTPNPKQPIRLQIALQQHDV | Aspergillus |
| | TGFEQSLLEMSTPDHPNYGKHFRTHDEMKRMLLPNENAVHAVREWLQDAGISDIEEDADW | ruber |
| | VRFHTTVDQANDLLDANFLWYAHKSHRNTARLRTLEYSIPDSIAPQVNVIQPTTRFGQIR | CBS |
| | ANRATHSSKPKGGLDELAISQAATADDDSICDQITTPHCLRKLYNVNGYKADPASGSKIG | 135680 |
| | FASFLEEYARYSDLVLFEENLAPFAEGENFTVVMYNGGKNDQNSKSDSGEANLDLQYIVG | (21) |
| | MSAGAPVTEFSTAGRAPVIPDLDQPDPSAGTNEPYLEFLQNVLHMDQEHLPQVISTSYGE | |
| | NEQTIPEKYARTVCNMYAQLGSRGVSVIFSSGDSGVGSACMTNDGTNRTHFPPQFPASCP | |
| | WVTSVGATEKMAPEQATYFSSGGFSDLFPRPKYQDAAVSSYLQTLGSRYQGLYNGSNRAF | |
| | PDVSAQGTNFAVYDKGRLGQFDGTSCSAPAFSGIIALLNDVRLQNNKPVLGFLNPWLYGA | |
| | GSKGLNDVVHGGSTGCDGQERFAGKANGSPVVPYASWNATQGWDPVTGLGTPDFGKLKDL | |
| | ALSA | |
| 36 | MLPSLVNNGALSLAVLSLLTSSVAGEVFEKLSAVPKGWHFSHAAQADAPINLKIALKQHD | Aspergillus |
| | VEGFEQALLDMSTPGHENYGKHFHEHDEMKRMLLPSDSAVDAVQTWLTSAGITDYDLDAD | terreus |
| | WINLRTTVEHANALLDTQFGWYENEVRHITRLRTLQYSIPETVAAHINMVQPTTRFGQIR | NIH2624 |
| | PDRATFHAHHTSDARILSALAAASNSTSCDSVITPKCLKDLYKVGDYEADPDSGSQVAFA | (22) |
| | SYLEEYARYADMVKFQNSLAPYAKGQNFSVVLYNGGVNDQSSSADSGEANLDLQTIMGLS | |
| | APLPITEYITGGRGKLIPDLSQPNPNDNSNEPYLEFLQNILKLDQDELPQVISTSYGEDE | |
| | QTIPRGYAESVCNMLAQLGSRGVSVVFSSGDSGVGAACQTNDGRNQTHFNPQFPASCPWV | |
| | TSVGATTKTNPEQAVYFSSGGFSDFWKRPKYQDEAVAAYLDTLGDKFAGLFNKGGRAFPD | |
| | VAAQGMNYAIYDKGTLGRLDGTSCSAPAFSAIISLLNDARLREGKPTMGFLNPWLYGEGR | |
| | EALNDVVVGGSKGCDGRDRFGGKPNGSPVVPFASWNATQGWDPVTGLGTPNFAKMLELAP | |
| 37 | MIASLFNRRALTLALLSLFASSATADVFESLSAVPQGWRYSRTPSANQPLKLQIALAQGD | Penicillium |
| | VAGFEAAVIDMSTPDHPSYGNHFNTHEEMKRMLQPSAESVDSIRNWLESAGISKIEQDAD | digitatum |
| | WMTFYTTVKTANELLAANFQFYINGVKKIERLRTLKYSVPDALVSHINMIQPTTRFGQLR | Pd1 (23) |
| | AQRAILHTEVKDNDEAFRSNAMSANPDCNSIITPQCLKDLYSIGDYEADPTNGNKVAFAS | |
| | YLEEYARYSDLALFEKNIAPFAKGQNFSVVQYNGGGNDQQSSSGSSEANLDLQYIVGVSS | |
| | PVPVTEFSTGGRGELVPDLDQPNPNDNNNEPYLEFLQNVLKLHKKDLPQVISTSYGEDEQ | |
| | SVPEKYARAVCNLYSQLGSRGVSVIFSSGDSGVGAACQTNDGRNATHFPPQFPAACPWVT | |
| | SVGATTHTAPERAVYFSSGGFSDLWDRPTWQEDAVSEYLENLGDRWSGLFNPKGRAFPDV | |
| | AAQGENYAIYDKGSLISVDGTSCSAPAFAGVIALLNDARIKANRPPMGFLNPWLYSEGRS | |
| | GLNDIVNGGSTGCDGHGRFSGPTNGGTSIPGASWNATKGWDPVSGLGSPNFAAMRKLANA | |
| | E | |
| 38 | MHVPLLNQGALSLAVVSLLASTVSAEVFDKLVAVPEGWRFSRTPSGDQPIRLQVALTQGD | Penicillium |
| | VEGFEKAVLDMSTPDHPNYGKHFKSHEEVKRMLQPAGESVEATHQWLEKAGITHIQQDAD | oxalicum |
| | WMTFYTTVEKANNLLDANFQYYLNENKQVERLRTLEYSVPDELVSHINLVTPTTRFGQLH | 114-2 |
| | AEGVTLHGKSKDVDEQFRQAATSPSSDCNSAITPQCLKDLYKVGDYKASASNGNKVAFTS | (24) |
| | YLEQYARYSDLALFEQNIAPYAQGQNFTVIQYNGGLNDQSSPADSSEANLDLQYIIGTSS | |
| | PVPVTEFSTGGRGPLVPDLDQPDINDNNNEPYLDFLQNVIKMSDKDLPQVISTSYGEDEQ | |
| | SVPASYARSVCNLIAQLGGRGVSVIFSSGDSGVGSACQTNDGKNTTRFPAQFPAACPWVT | |
| | SVGATTGISPERGVFFSSGGFSDLWSRPSWQSHAVKAYLHKLGKRQDGLFNREGRAFPDV | |
| | SAQGENYAIYAKGRLGKVDGTSCSAPAFAGLVSLLNDARIKAGKSSLGFLNPWLYSHPDA | |
| | LNDITVGGSTGCDGNARFGGRPNGSPVVPYASWNATEGWDPVTGLGTPNFQKLLKSAVKQ | |
| | K | |
| 39 | MIASLFSRGALSLAVLSLLASSAAADVFESLSAVPQGWRYSRRPRADQPLKLQIALTQGD | Penicillium |
| | TAGFEEAVMEMSTPDHPSYGHHFTTHEEMKRMLQPSAESAESIRDWLEGAGITRIEQDAD | rogueforti |
| | WMTFYTTVETANELLAANFQFYVSNVRHIERLRTLKYSVPKALVPHINMIQPTTRFGQLR | FM 164 |
| | AHRGILHGQVKESDEAFRSNAVSAQPDCNSIITPQCLKDIYNIGDYQANDTNGNKVGFAS | (25) |
| | YLEEYARYSDLALFEKNIAPSAKGQNFSVTRYNGGLNDQSSSGSSSEANLDLQYIVGVSS | |
| | PVPVTEFSVGGRGELVPDLDQPDPNDNNNEPYLEFLQNVLKLDKKDLPQVISTSYGEDEQ | |
| | SIPEKYARSVCNLYSQLGSRGVSVIFSSGDSGVGSACLTNDGRNATRFPPQFPAACPWVT | |
| | SVGATTHTAPEQAVYFSSGGFSDLWARPKWQEEAVSEYLEILGNRWSGLFNPKGRAFPDV | |
| | TAQGRNYAIYDKGSLTSVDGTSCSAPAFAGVVALLNDARLKVNKPPMGFLNPWLYSTGRA | |
| | GLKDIVDGGSTGCDGKSRFGGANNGGPSIPGASWNATKGWDPVSGLGSPNFATMRKLANA | |
| | E | |
| 40 | MIASLFNRGALSLAVLSLLASSASADVFESLSAVPQGWRYSRRPRADQPLKLQIALAQGD | Penicillium |
| | TAGFEEAVMDMSTPDHPSYGNHFHTHEEMKRMLQPSAESADSIRDWLESAGINRIEQDAD | rubens |
| | WMTFYTTVETANELLAANFQFYANSAKHIERLRTLQYSVPEALMPHINMIQPTTRFGQLR | Wisconsin |
| | VQGAILHTQVKETDEAFRSNAVSTSPDCNSIITPQCLKNMYNVGDYQADDDNGNKVGFAS | 54-1255 |
| | YLEEYARYSDLELFEKNVAPFAKGQNFSVIQYNGGLNDQHSSASSSEANLDLQYIVGVSS | (26) |
| | PVPVTEFSVGGRGELVPDLDQPDPNDNNNEPYLEFLQNVLKMEQQDLPQVISTSYGENEQ | |
| | SVPEKYARTVCNLFSQLGSRGVSVIFASGDSGVGAACQTNDGRNATRFPAQFPAACPWVT | |
| | SVGATTHTAPEKAVYFSSGGFSDLWDRPKWQEDAVSDYLDTLGDRWSGLFNPKGRAFPDV | |
| | SAQGQNYAIYDKGSLTSVDGTSCSAPAFAGVIALLNDARLKANKPPMGFLNPWLYSTGRD | |
| | GLNDIVHGGSTGCDGNARFGGPGNGSPRVPGASWNATKGWDPVSGLGSPNFATMRKLANG | |
| | E | |
| 41 | MLSSTLYAGLLCSLAAPALGVVHEKLSAVPSGWTLVEDASESDTTTLSIALARQNLDQLE | Neosartorya |
| | SKLTTLATPGNAEYGKWLDQSDIESLFPTASDDAVIQWLKDAGVTQVSRQGSLVNFATTV | fischeri |
| | GTANKLFDTKFSYYRNGASQKLRTTQYSIPDSLTESIDLIAPTVFFGKEQDSALPPHAVK | NRRL 181 |
| | LPALPRRAATNSSCANLITPDCLVEMYNLGDYKPDASSGSRVGFGSFLNQSANYADLAAY | (27) |
| | EQLFNIPPQNFSVELINGGANDQNWATASLGEANLDVELIVAVSHALPVVEFITGGSPPF | |
| | VPNVDEPTAADNQNEPYLQYYEYLLSKPNSHLPQVISNSYGDDEQTVPEYYARRVCNLIG | |
| | LMGLRGITVLESSGDTGIGSACMSNDGTNTPQFTPTFPGTCPFITAVGGTQSYAPEVAWD | |
| | ASSGGFSNYFSRPWYQYFAVENYLNNHITKDTKKYYSQYTNFKGRGFPDVSAHSLTPDYE | |
| | VVLTGKHYKSGGTSAACPVFAGIVGLLNDARLRAGKSTLGFLNPLLYSILAEGFTDITAG | |
| | SSIGCNGINPQTGKPVPGGGIIPYAHWNATAGWDPVTGLGVPDFMKLKELVLSL | |
| 42 | MLSSTLYAGWLLSLAAPALCVVQEKLSAVPSGWTLIEDASESDTITLSIALARQNLDQLE | Aspergillus |
| | SKLTTLATPGNPEYGKWLDQSDIESLFPTASDDAVLQWLKAAGITQVSRQGSLVNFATTV | fumigatus |
| | GTANKLFDTKFSYYRNGASQKLRTTQYSIPDHLTESIDLIAPTVFFGKEQNSALSSHAVK | CAE17675 |
| | LPALPRRAATNSSCANLITPDCLVEMYNLGDYKPDASSGSRVGFGSFLNESANYADLAAY | (28) |
| | EQLFNIPPQNFSVELINRGVNDQNWATASLGEANLDVELIVAVSHPLPVVEFITGALPPV | |
| | LRVLALQTQLPSSSGDFQLTVPEYYARRVCNLIGLMGLRGITVLESSGDTGIGSACMSND | |
| | GTNKPQFTPTFPGTCPFITAVGGTQSYAPEVAWDGSSGGFSNYFSRPWYQSFAVDNYLNN | |
| | HITKDTKKYYSQYTNFKGRGFPDVSAHSLTPYYEVVLTGKHYKSGGTSAASPVFAGIVGL | |
| | LNDARLRAGKSTLGFLNPLLYSILAEGFTDITAGSSIGCNGINPQTGKPVPGGGIIPYAH | |
| | WNATAGWDPVTGLGVPDFMKLKELVLSL | |
| 43 | QEPSSCKGTLVFEGETFNVFQPDCLRTEYSVDGYTPSVKSGSRIGFGSFLNESASFADQA | Trichoderma |
| | LFEKHFNIPSQNFSVVLINGGTDLPQPPSDANDGEANLDAQTILTIAHPLPITEFITAGS | reesei |
| | PPYFPDPVEPAGTPNENEPYLQYYEFLLSKSNAEIPQVITNSYGDEEQTVPRSYAVRVCN | QM6a |
| | LIGLLGLRGISVLHSSGDEGVGASCVATNSTTPQFNPIFPATCPYVTSVGGTVSFNPEVA | (29) |
| | WAGSSGGFSYYFSRPWYQQEAVGTYLEKYVSAETKKYYGPYVDFSGRGFPDVAAHSVSPD | |
| | YPVFQGGELTPSGGTSAASPVVAAIVALLNDARLREGKPTLGFLNPLIYLHASKGFTDIT | |
| | SGQSEGCNGNNTQTGSPLPGAGFIAGAHWNATKGWDPTTGFGVPNLKKLLALVRF | |
| 44 | CDSIITPTCLKELYNIGDYQADANSGSKIAFASYLEEYARYADLENFENYLAPWAKGQNF | Aspergillus |
| | SVTTFNGGLNDQNSSSDSGEANLDLQYILGVSAPLPVTEFSTGGRGPLVPDLTQPDPNSN | oryzae |
| | SNEPYLEFFQNVLKLDQKDLPQVISTSYGENEQEIPEKYARTVCNLIAQLGSRGVSVLFS | RIB40 |
| | SGDSGVGEGCMTNDGTNRTHFPPQFPAACPWVTSVGATFKTTPERGTYFSSGGFSDYWPR | (30) |
| | PEWQDEAVSSYLETIGDTFKGLYNSSGRAFPDVAAQGMNFAVYDKGTLGEFDGTSASAPA | |
| | FSAVIALLNDARLRAGKPTLGFLNPWLYKTGRQGLQDITLGASIGCTGRARFGGAPDGGP | |
| | VVPYASWNATQGWDPVTGLGTPDFAELKKLA | |
| 45 | CDATITPQCLKTLYKIDYKADPKSGSKVAFASYLEQYARYNDLALFEKAFLPEAVGQNFS | Phaeosphaeria |
| | VVQFSGGLNDQNTTQDSGEANLDLQYIVGVSAPLPVTEFSTGGRGPWVADLDQPDEADSA | nodorum |
| | NEPYLEFLQGVLKLPQSELPQVISTSYGENEQSVPKSYALSVCNLFAQLGSRGVSVIFSS | SN15 |
| | GDSGPGSACQSNDGKNTTKFQPQYPAACPFVTSVGSTRYLNETATGFSSGGFSDYWKRPS | (31) |
| | YQDDAVKAYFHHLGEKFKPYFNRHGRGFPDVATQGYGFRVYDQGKLKGLQGTSASAPAFA | |
| | GVIGLLNDARLKAKKPTLGFLNPLLYSNSDALNDIVLGGSKGCDGHARFNGPPNGSPVIP | |
| | YAGWNATAGWDPVTGLGTPNFPKLLKAA | |
| 46 | VFQPDCLRTEYSVNGYKPSAKSGSRIGFGSFLNQSASSSDLALFEKHFGFASQGFSVELI | Trichoderma |
| | NGGSNPQPPTDANDGEANLDAQNIVSFVQPLPITEFIAGGTAPYFPDPVEPAGTPDENEP | atroviride |
| | YLEYYEYLLSKSNKELPQVITNSYGDEEQTVPQAYAVRVCNLIGLMGLRGISILESSGDE | IMI |
| | GVGASCLATNSTTTPQFNPIFPATCPYVTSVGGTVSFNPEVAWDGSSGGFSYYFSRPWYQ | 206040 |
| | EAAVGTYLNKYVSEETKEYYKSYVDFSGRGFPDVAAHSVSPDYPVFQGGELTPSGGTSAA | (32) |
| | SPIVASVIALLNDARLRAGKPALGFLNPLIYGYAYKGFTDITSGQAVGCNGNNTQTGGPL | |
| | PGAGVIPGAFWNATKGWDPTTGFGVPNFKKLLELV | |
| 47 | CRSLVTTACLRELYGLGDRVTQARDDNRIGVSGFLEEYAQYRDLELFLSRFEPSAKGFNF | Arthroderma |
| | SEGLIAGGKNTQGGPGSSTEANLDMQYVVGLSHKAKVTYYSTAGRGPLIPDLSQPSQASN | benhamiae |
| | NNEPYLEQLRYLVKLPKNQLPSVLTTSYGDTEQSLPASYTKATCDLFAQLGTMGVSVIFS | CBS |
| | SGDTGPGSSCQTNDGKNATRFNPIYPASCPFVTSIGGTVGTGPERAVSFSSGGFSDRFPR | 112371 |
| | PQYQDNAVKDYLKILGNQWSGLFDPNGRAFPDIAAQGSNYAVYDKGRMTGVSGTSASAPA | (33) |
| | MAAIIAQLNDFRLAKGSPVLGFLNPWIYSKGFSGFTDIVDGGSRGCTGYDIYSGLKAKKV | |
| | PYASWNATKGWDPVTGFGTPNFQALTKVL | |
| 48 | CQTSITPSCLKQMYNIGDYTPKVESGSTIGFSSFLGESAIYSDVFLFEEKFGIPTQNFTT | Fusarium |
| | VLINNGTDDQNTAHKNFGEADLDAENIVGIAHPLPFTQYITGGSPPFLPNIDQPTAADNQ | graminearum |
| | NEPYVPFFRYLLSQKEVPAVVSTSYGDEEDSVPREYATMTCNLIGLLGLRGISVIFSSGD | PH-1 |
| | IGVGAGCLGPDHKTVEFNAIFPATCPYLTSVGGTVDVTPEIAWEGSSGGFSKYFPRPSYQ | (34) |
| | DKAVKTYMKTVSKQTKKYYGPYTNWEGRGFPDVAGHSVSPNYEVIYAGKQSASGGTSAAA | |
| | PVWAAIVGLLNDARFRAGKPSLGWLNPLVYKYGPKVLTDITGGYAIGCDGNNTQSGKPEP | |
| | AGSGIVPGARWNATAGWDPVTGYGTPDFGKLKDLVLS | |
| 49 | CDLVITPPCLEAAYNYKNYMPDPNSGSRVSFTSFLEQAAQQSDLTKFLSLTGLDRLRPPS | Acremonium |
| | SKPASFDTVLINGGETHQGTPPNKTSEANLDVQWLAAVIKARLPITQWITGGRPPFVPNL | alcalophilum |
| | RLRHEKDNTNEPYLEFFEYLVRLPARDLPQVISNSYAEDEQTVPEAYARRVCNLIGIMGL | (35) |
| | RGVTVLTASGDSGVGAPCRANDGSDRLEFSPQFPTSCPYITAVGGTEGWDPEVAWEASSG | |
| | GFSHYFLRPWYQANAVEKYLDEELDPATRAYYDGNGFVQFAGRAYPDLSAHSSSPRYAYI | |
| | DKLAPGLTGGTSASCPVVAGIVGLLNDARLRRGLPTMGFINPWLYTRGFEALQDVTGGRA | |
| | SGCQGIDLQRGTRVPGAGIIPWASWNATPGWDPATGLGLPDFWAMRGL | |
| 50 | CATIITPPCLETAYNYKGYIPDPKSGSRVSFTSFLEQAAQQADLTKFLSLTRLEGFRTPA | Sodiomyces |
| | SKKKTFKTVLINGGESHEGVHKKSKTSEANLDVQWLAAVTQTKLPITQWITGGRPPFVPN | alkalinus |
| | LRIPTPEANTNEPYLEFLEYLFRLPDKDLPQVISNSYAEDEQSVPEAYARRVCGLLGIMG | (36) |
| | LRGVTVLTASGDSGVGAPCRANDGSGREEFSPQFPSSCPYITTVGGTQAWDPEVAWKGSS | |
| | GGFSNYFPRPWYQVAAVEKYLEEQLDPAAREYYEENGFVRFAGRAFPDLSAHSSSPKYAY | |
| | VDKRVPGLTGGTSASCPVVAGIVGLLNDARLRRGLPTMGFINPWLYAKGYQALEDVTGGA | |
| | AVGCQGIDIQTGKRVPGAGIIPGASWNATPDWDPATGLGLPNFWAMRELA | |
| 51 | CADTITLSCLKEMYNFGNYTPSASSGSKLGFASFLNESASYSDLAKFERLFNLPSQNFSV | Aspergillus |
| | ELINGGVNDQNQSTASLTEADLDVELLVGVGHPLPVTEFITSGEPPFIPDPDEPSAADNE | kawachii |
| | NEPYLQYYEYLLSKPNSALPQVISNSYGDDEQTVPEYYAKRVCNLIGLVGLRGISVLESS | IFO 4308 |
| | GDEGIGSGCRTTDGTNSTQFNPIFPATCPYVTAVGGTMSYAPEIAWEASSGGFSNYFERA | (37) |
| | WFQKEAVQNYLANHITNETKQYYSQFANFSGRGFPDVSAHSFEPSYEVIFYGARYGSGGT | |
| | SAACPLFSALVGMLNDARLRAGKSTLGELNPLLYSKGYKALTDVTAGQSIGCNGIDPQSD | |
| | EAVAGAGIIPWAHWNATVGWDPVTGLGLPDFEKLRQLVLS | |
| 52 | CQTSITPACLKQMYNVGNYTPSVAHGSRVGEGSFLNQSAIFDDLFTYEKVNDIPSQNFTK | Talaromyces |
| | VIIANASNSQDASDGNYGEANLDVQNIVGISHPLPVTEFLTGGSPPFVASLDTPTNQNEP | stipitatus |
| | YIPYYEYLLSQKNEDLPQVISNSYGDDEQSVPYKYAIRACNLIGLTGLRGISVLESSGDL | ATCC10500 |
| | GVGAGCRSNDGKNKTQFDPIFPATCPYVTSVGGTQSVTPEIAWVASSGGFSNYFPRTWYQ | (38) |
| | EPAIQTYLGLLDDETKTYYSQYTNFEGRGFPDVSAHSLTPDYQVVGGGYLQPSGGTSAAS | |
| | PVFAGIIALLNDARLAAGKPTLGFLNPFFYLYGYKGLNDITGGQSVGCNGINGQTGAPVP | |
| | GGGIVPGAAWNSTTGWDPATGLGTPDFQKLKELVLS | |
| 53 | CQTSITPSCLKQMYNIGDYTPDAKSGSEIGFSSFLGQAAIYSDVFKFEELFGIPKQNYTT | Fusarium |
| | ILINNGTDDQNTAHGNFGEANLDAENIVGIAHPLPFKQYITGGSPPFVPNIDQPTEKDNQ | oxysporum |
| | NEPYVPFFRYLLGQKDLPAVISTSYGDEEDSVPREYATLTCNMIGLLGLRGISVIFSSGD | f. sp. |
| | IGVGSGCLAPDYKTVEFNAIFPATCPYLTSVGGTVDVTPEIAWEGSSGGFSKYFPRPSYQ | Cubense |
| | DKAIKKYMKTVSKETKKYYGPYTNWEGRGFPDVAGHSVAPDYEVIYNGKQARSGGTSAAA | race 4 |
| | PVWAAIVGLLNDARFKAGKKSLGWLNPLIYKHGPKVLTDITGGYAIGCDGNNTQSGKPEP | (39) |
| | AGSGLVPGARWNATAGWDPTTGYGTPNFQKLKDLVLS | |
| 54 | VFQPDCLRTEYNVNGYTPSAKSGSRIGEGSFLNQSASFSDLALFEKHFGESSQNFSVVLI | Trichoderma |
| | NGGTDLPQPPSDDNDGEANLDVQNILTIAHPLPITEFITAGSPPYFPDPVEPAGTPDENE | virens |
| | PYLQYFEYLLSKPNRDLPQVITNSYGDEEQTVPQAYAVRVCNLIGLMGLRGISILESSGD | Gv29-8 |
| | EGVGASCVATNSTTPQFNPIFPATCPYVTSVGGTVNENPEVAWDGSSGGESYYFSRPWYQ | (40) |
| | EEAVGNYLEKHVSAETKKYYGPYVDFSGRGFPDVAAHSVSPDYPVFQGGQLTPSGGTSAA | |
| | SPVVASIIALLNDARLREGKPTLGFLNPLIYQYAYKGFTDITSGQSDGCNGNNTQTDAPL | |
| | PGAGVVLGAHWNATKGWDPTTGFGVPNFKKLLELI | |
| 55 | QIFHPDCLKTKYGVDGYAPSPRCGSRIGFGSFLNETASYSDLAQFEKYFDLPNQNLSTLL | Trichoderma |
| | INGAIDVQPPSNKNDSEANMDVQTILTFVQPLPITEFVVAGIPPYIPDAALPIGDPVQNE | atrovirde |
| | PWLEYFEFLMSRTNAELPQVIANSYGDEEQTVPQAYAVRVCNQIGLLGLRGISVIASSGD | IMI |
| | TGVGMSCMASNSTTPQFNPMFPASCPYITTVGGTQHLDNEIAWELSSGGFSNYFTRPWYQ | 206040 |
| | EDAAKTYLERHVSTETKAYYERYANFLGRGFPDVAALSLNPDYPVIIGGELGPNGGTSAA | (41) |
| | APVVASIIALLNDARLCLGKPALGFLNPLIYQYADKGGFTDITSGQSWGCAGNTTQTGPP | |
| | PPGAGVIPGAHWNATKGWDPVTGFGTPNFKKLLSLALS | |
| 56 | TVITPDCLRDLYNTADYVPSATSRNAIGIAGYLDRSNRADLQTFFRRFRPDAVGFNYTTV | Agaricus |
| | QLNGGGDDQNDPGVEANLDIQYAAGIAFPTPATYWSTGGSPPFIPDTQTPTNTNEPYLDW | bisporus |
| | INFVLGQDEIPQVISTSYGDDEQTVPEDYATSVCNLFAQLGSRGVTVFFSSGDFGVGGGD | var. |
| | CLTNDGSNQVLFQPAFPASCPFVTAVGGTVRLDPEIAVSFSGGGFSRYFSRPSYQNQTVA | burnettii |
| | QFVSNLGNTFNGLYNKNGRAYPDLAAQGNGFQVVIDGIVRSVGGTSASSPTVAGIFALLN | JB137-58 |
| | DFKLSRGQSTLGFINPLIYSSATSGFNDIRAGTNPGCGTRGFTAGTGWDPVTGLGTPDFL | (42) |
| | RLQ | |
| 57 | GVTPLCLRTLYRVNYKPATTGNLVAFASFLEQYARYSDQQAFTQRVLGPGVPLQNFSVET | Magnaporthe |
| | VNGGANDQQSKLDSGEANLDLQYVMAMSHPIPILEYSTGGRGPLVPTLDQPNANNSSNEP | oryzae |
| | YLEFLTYLLAQPDSAIPQTLSVSYGEEEQSVPRDYAIKVCNMFMQLGARGVSVMFSSGDS | 70-15 |
| | GPGNDCVRASDNATFFGSTFPAGCPYVTSVGSTVGFEPERAVSFSSGGFSIYHARPDYQN | (43) |
| | EVVPKYIESIKASGYEKFFDGNGRGIPDVAAQGARFVVIDKGRVSLISGTSASSPAFAGM | |
| | VALVNAARKSKDMPALGFLNPMLYQNAAAMTDIVNGAGIGCRKQRTEFPNGARFNATAGW | |
| | DPVTGLGTPLFDKLLA | |
| 58 | CNASITPECLRALYNVGDYEADPSKKSLFGVCGYLEQYAKHDQLAKFEQTYAPYAIGADF | Togninia |
| | SVVTINGGGDNQTSTIDDGEANLDMQYAVSMAYKTPITYYSTGGRGPLVPDLDQPDPNDV | minima |
| | SNEPYLDFVSYLLKLPDSKLPQTITTSYGEDEQSVPRSYVEKVCTMFGALGARGVSVIFS | UCRPA7 |
| | SGDTGVGSACQTNDGKNTTRFLPIFPAACPYVTSVGGTRYVDPEVAVSFSSGGFSDIFPT | (44) |
| | PLYQKGAVSGYLKILGDRWKGLYNPHGRGFPDVSGQSVRYHVFDYGKDVMYSGTSASAPM | |
| | FAALVSLLNNARLAKKLPPMGFLNPWLYTVGFNGLTDIVHGGSTGCTGTDVYSGLPTPFV | |
| | PYASWNATVGWDPVTGLGTPLFDKLLNL | |
| 59 | CNKKITPDCLANLYNFKDYDASDANVTIGVSGFLEQYARFDDLKQFISTFQPKAAGSTFQ | Bipolaris |
| | VTSVNAGPFDQNSTASSVEANLDIQYTTGLVAPDIETRYFTVPGRGILIPDLDQPTESDN | maydis C5 |
| | ANEPYLDYFTYLNNLEDEELPDVLTTSYGESEQSVPAEYAKKVCNLIGQLGARGVSVIFS | (45) |
| | SGDTGPGSACQTNDGKNTTRFLPIFPASCPYVTSVGGTVGVEPEKAVSFSSGGFSDLWPR | |
| | PAYQEKAVSEYLEKLGDRWNGLYNPQGRGFPDVAAQGQGFQVFDKGRLISVGGTSASAPV | |
| | FASVVALLNNARKAAGMSSLGFLNPWIYEQGYKGLTDIVAGGSTGCTGRSIYSGLPAPLV | |
| | PYASWNATEGWDPVTGYGTPDFKQLLTLAT | |
| 60 | CDSIITPHCLKQLYNIGDYQADPKSGSKVGFASYLEEYARYADLERFEQHLAPNAIGQNF | Aspergillus |
| | SVVQFNGGLNDQLSLSDSGEANLDLQYILGVSAPVPVTEYSTGGRGELVPDLSSPDPNDN | kawachii |
| | SNEPYLDFLQGILKLDNSDLPQVISTSYGEDEQTIPVPYARTVCNLYAQLGSRGVSVIFS | IFO 4308 |
| | SGDSGVGAACLTNDGTNRTHFPPQFPASCPWVTSVGATSKTSPEQAVSFSSGGFSDLWPR | (46) |
| | PSYQQAAVQTYLTQHLGNKFSGLFNASGRAFPDVAAQGVNYAVYDKGMLGQFDGTSCSAP | |
| | TFSGVIALLNDARLRAGLPVMGFLNPFLYGVGSESGALNDIVNGGSLGCDGRNRFGGTPN | |
| | GSPVVPFASWNATTGWDPVSGLGTPDFAKLRGV | |
| 61 | CEKAITPSCLADLYNTEGYKASNRSGSKVAFASFLEEYARYDDLAEFEETYAPYAIGQNF | Aspergillus |
| | SVISINGGLNDQDSTADSGEANLDLQYIIGVSSPLPVTEFTTGGRGKLIPDLSSPDPNDN | nidulans |
| | TNEPFLDFLEAVLKLDQKDLPQVISTSYGEDEQTIPEPYARSVCNLYAQLGSRGVSVLFS | FGSC A4 |
| | SGDSGVGAACQTNDGKNTTHFPPQFPASCPWVTAVGGTNGTAPESGVYFSSGGFSDYWAR | (47) |
| | RAYQNAAVESYLRKLGSTQAQYFNRSGRAFPDVAAQAQNFAVVDKGRVGLFDGTSCSSPV | |
| | FAGIVALLNDVRLKAGLPVLGFLNPWLYQDGLNGLNDIVDGGSTGCDGNNRFNGSPNGSP | |
| | VIPYAGWNATEGWDPVTGLGTPDFAKLKALVL | |
| 62 | CDQITTPHCLRKLYNVNGYKADPASGSKIGFASFLEEYARYSDLVLFEENLAPFAEGENF | Aspergillus |
| | TVVMYNGGKNDQNSKSDSGEANLDLQYIVGMSAGAPVTEFSTAGRAPVIPDLDQPDPSAG | ruber |
| | TNEPYLEFLQNVLHMDQEHLPQVISTSYGENEQTIPEKYARTVCNMYAQLGSRGVSVIFS | CBS |
| | SGDSGVGSACMTNDGTNRTHFPPQFPASCPWVTSVGATEKMAPEQATYFSSGGFSDLFPR | 135680 |
| | PKYQDAAVSSYLQTLGSRYQGLYNGSNRAFPDVSAQGTNFAVYDKGRLGQFDGTSCSAPA | (48) |
| | FSGIIALLNDVRLQNNKPVLGFLNPWLYGAGSKGLNDVVHGGSTGCDGQERFAGKANGSP | |
| | VVPYASWNATQGWDPVTGLGTPDFGKLKDLAL | |
| 63 | CDSVITPKCLKDLYKVGDYEADPDSGSQVAFASYLEEYARYADMVKFQNSLAPYAKGQNF | Aspergillus |
| | SVVLYNGGVNDQSSSADSGEANLDLQTIMGLSAPLPITEYITGGRGKLIPDLSQPNPNDN | terreus |
| | SNEPYLEFLQNILKLDQDELPQVISTSYGEDEQTIPRGYAESVCNMLAQLGSRGVSVVFS | NIH2624 |
| | SGDSGVGAACQTNDGRNQTHFNPQFPASCPWVTSVGATTKTNPEQAVYFSSGGFSDFWKR | (49) |
| | PKYQDEAVAAYLDTLGDKFAGLFNKGGRAFPDVAAQGMNYAIYDKGTLGRLDGTSCSAPA | |
| | FSAIISLLNDARLREGKPTMGFLNPWLYGEGREALNDVVVGGSKGCDGRDRFGGKPNGSP | |
| | VVPFASWNATQGWDPVTGLGTPNFAKMLELA | |
| 64 | CNSIITPQCLKDLYSIGDYEADPTNGNKVAFASYLEEYARYSDLALFEKNIAPFAKGQNF | Penicillium |
| | SVVQYNGGGNDQQSSSGSSEANLDLQYIVGVSSPVPVTEFSTGGRGELVPDLDQPNPNDN | digitatum |
| | NNEPYLEFLQNVLKLHKKDLPQVISTSYGEDEQSVPEKYARAVCNLYSQLGSRGVSVIFS | Pd1 |
| | SGDSGVGAACQTNDGRNATHFPPQFPAACPWVTSVGATTHTAPERAVYFSSGGFSDLWDR | (50) |
| | PTWQEDAVSEYLENLGDRWSGLFNPKGRAFPDVAAQGENYAIYDKGSLISVDGTSCSAPA | |
| | FAGVIALLNDARIKANRPPMGFLNPWLYSEGRSGLNDIVNGGSTGCDGHGRFSGPTNGGT | |
| | SIPGASWNATKGWDPVSGLGSPNFAAMRKLA | |
| 65 | CNSAITPQCLKDLYKVGDYKASASNGNKVAFTSYLEQYARYSDLALFEQNIAPYAQGQNF | Penicillium |
| | TVIQYNGGLNDQSSPADSSEANLDLQYIIGTSSPVPVTEFSTGGRGPLVPDLDQPDINDN | oxalicum |
| | NNEPYLDFLQNVIKMSDKDLPQVISTSYGEDEQSVPASYARSVCNLIAQLGGRGVSVIFS | 114-2 |
| | SGDSGVGSACQTNDGKNTTRFPAQFPAACPWVTSVGATTGISPERGVFFSSGGFSDLWSR | (51) |
| | PSWQSHAVKAYLHKLGKRQDGLFNREGRAFPDVSAQGENYAIYAKGRLGKVDGTSCSAPA | |
| | FAGLVSLLNDARIKAGKSSLGFLNPWLYSHPDALNDITVGGSTGCDGNARFGGRPNGSPV | |
| | VPYASWNATEGWDPVTGLGTPNFQKLLKSAV | |
| 66 | CNSIITPQCLKDIYNIGDYQANDTNGNKVGFASYLEEYARYSDLALFEKNIAPSAKGQNF | Penicillium |
| | SVTRYNGGLNDQSSSGSSSEANLDLQYIVGVSSPVPVTEFSVGGRGELVPDLDQPDPNDN | roqueforti |
| | NNEPYLEFLQNVLKLDKKDLPQVISTSYGEDEQSIPEKYARSVCNLYSQLGSRGVSVIFS | FM164 |
| | SGDSGVGSACLTNDGRNATRFPPQFPAACPWVTSVGATTHTAPEQAVYFSSGGFSDLWAR | (52) |
| | PKWQEEAVSEYLEILGNRWSGLFNPKGRAFPDVTAQGRNYAIYDKGSLTSVDGTSCSAPA | |
| | FAGVVALLNDARLKVNKPPMGFLNPWLYSTGRAGLKDIVDGGSTGCDGKSRFGGANNGGP | |
| | SIPGASWNATKGWDPVSGLGSPNFATMRKLA | |
| 67 | CNSIITPQCLKNMYNVGDYQADDDNGNKVGFASYLEEYARYSDLELFEKNVAPFAKGQNF | Penicillium |
| | SVIQYNGGLNDQHSSASSSEANLDLQYIVGVSSPVPVTEFSVGGRGELVPDLDQPDPNDN | rubens |
| | NNEPYLEFLQNVLKMEQQDLPQVISTSYGENEQSVPEKYARTVCNLFSQLGSRGVSVIFA | Wisconsin |
| | SGDSGVGAACQTNDGRNATRFPAQFPAACPWVTSVGATTHTAPEKAVYFSSGGFSDLWDR | 54-1255 |
| | PKWQEDAVSDYLDTLGDRWSGLFNPKGRAFPDVSAQGQNYAIYDKGSLTSVDGTSCSAPA | (53) |
| | FAGVIALLNDARLKANKPPMGFLNPWLYSTGRDGLNDIVHGGSTGCDGNARFGGPGNGSP | |
| | RVPGASWNATKGWDPVSGLGSPNFATMRKLA | |
| 68 | CANLITPDCLVEMYNLGDYKPDASSGSRVGFGSFLNQSANYADLAAYEQLFNIPPQNFSV | Neosartorya |
| | ELINGGANDQNWATASLGEANLDVELIVAVSHALPVVEFITGGSPPFVPNVDEPTAADNQ | fischeri |
| | NEPYLQYYEYLLSKPNSHLPQVISNSYGDDEQTVPEYYARRVCNLIGLMGLRGITVLESS | NRRL 181 |
| | GDTGIGSACMSNDGTNTPQFTPTFPGTCPFITAVGGTQSYAPEVAWDASSGGFSNYFSRP | (54) |
| | WYQYFAVENYLNNHITKDTKKYYSQYTNFKGRGFPDVSAHSLTPDYEVVLTGKHYKSGGT | |
| | SAACPVFAGIVGLLNDARLRAGKSTLGFLNPLLYSILAEGFTDITAGSSIGCNGINPQTG | |
| | KPVPGGGIIPYAHWNATAGWDPVTGLGVPDFMKLKELVLS | |
| 69 | CANLITPDCLVEMYNLGDYKPDASSGSRVGFGSFLNESANYADLAAYEQLFNIPPQNFSV | Aspergillus |
| | ELINRGVNDQNWATASLGEANLDVELIVAVSHPLPVVEFITGALPPVLRVLALQTQLPSS | fumigatus |
| | SGDFQLTVPEYYARRVCNLIGLMGLRGITVLESSGDTGIGSACMSNDGTNKPQFTPTFPG | CAE17675 |
| | TCPFITAVGGTQSYAPEVAWDGSSGGFSNYFSRPWYQSFAVDNYLNNHITKDTKKYYSQY | (55) |
| | TNFKGRGFPDVSAHSLTPYYEVVLTGKHYKSGGTSAASPVFAGIVGLLNDARLRAGKSTL | |
| | GFLNPLLYSILAEGFTDITAGSSIGCNGINPQTGKPVPGGGIIPYAHWNATAGWDPVTGL | |
| | GVPDFMKLKELVLS | |

In accordance with an aspect of the instant invention, an isolated polypeptide is described having proline specific endopeptidase activity having a polypeptide which is at least 70% sequence identity to one of SEQ ID NO: 4, SEQ ID NO: 6 and SEQ ID NO: 8 or a fragment thereof. More, preferably the polypeptide has at least 80% sequence identity to one of SEQ ID NO: 4, SEQ ID NO: 6 and SEQ ID NO: 8 or a fragment thereof. Still more preferably, the polypeptide has at least 90% sequence identity to one of SEQ ID NO: 4, SEQ ID NO: 6 and SEQ ID NO: 8 or a fragment thereof. In yet more preferred embodiments, the polypeptide has at least 95% sequence identity to one of SEQ ID NO: 4, SEQ ID NO:6 and SEQ ID NO:8 or a fragment thereof. In still more preferred embodiments, the polypeptide has at least 99% sequence identity to one of SEQ ID NO: 4, SEQ ID NO:6 and SEQ ID NO:8. In the most preferred embodiments, the polypeptide is a sequence according to one of SEQ ID NO:4, SEQ ID NO:6 and SEQ ID NO:8 or a fragment thereof.

In accordance with an aspect of the present invention, a method for the reduction or prevention of haze in a beverage is presented having the step of adding an isolated polypeptide having proline specific endopeptidase as described above to the beverage. Preferably, the beverage contains at least one protein. More preferably, the protein comprises hordein. Still more preferably, the beverage further comprises polyphenols. Preferably, the beverage has a pH of less than 7.

Preferably, the beverage is a fruit juice. In other preferred embodiments, the beverage is a wine. In yet other preferred embodiments, the beverage is a beer. Preferably, the isolated polypeptide is added to a mash.

Preferably, the isolated polypeptide is added before haze formation. In other preferred embodiments, the isolated polypeptide is added after haze formation.

In other preferred embodiments, the method of haze reduction has the further step of adding a second isolated polypeptide having proline specific endopeptidase activity as described above wherein the second isolated polypeptide is different than the isolated polypeptide. In still more preferred embodiments, the isolated polypeptide is a polypeptide according to SEQ ID NO:4 or a fragment thereof and the second isolated polypeptide is a polypeptide according to SEQ ID NO:8 or a fragment thereof.

In another aspect of the present invention, a method for forming a protein hydrolysate is presented having the step of adding to a protein substrate an isolated polypeptide having endopeptidase as described above. Preferably, the method includes the further step of adding a protease wherein the protease is different than the isolated polypeptide. More preferably, the protease is a second isolated polypeptide having proline specific endopeptidase activity as described above. Still more preferably, the isolated polypeptide is a polypeptide according to SEQ ID NO:4 or a fragment thereof and the second isolated polypeptide is a polypeptide according to SEQ ID NO:8 or a fragment thereof.

In other preferred embodiments, the protease is an exopeptidase. More preferably, the exopeptidase is a tripeptidyl aminopeptidase. Yet more preferably, the tripeptidyl aminopeptidase is a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Preferably, the polypeptide has at least 70% sequence identity to SEQ ID NO:15, SEQ ID NO:16 or SEQ ID NO:17 or a fragment thereof.

More preferably, the polypeptide has at least 80% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Preferably, the polypeptide has at least 80% sequence identity to SEQ ID NO:15, SEQ ID NO:16 or SEQ ID NO:17 or a fragment thereof.

More preferably, the polypeptide has at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Preferably, the polypeptide has at least 90% sequence identity to SEQ ID NO:15, SEQ ID NO:16 or SEQ ID NO:17 or a fragment thereof.

Still more preferably, the polypeptide has at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Preferably, the polypeptide has at least 90% sequence identity to SEQ ID NO:15, SEQ ID NO:16 or SEQ ID NO:17 or a fragment thereof.

Preferably, the polypeptide has at least 99% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

Still more preferably, the tripeptidyl aminopeptidase has at least 99% sequence identity to SEQ ID NO:15, SEQ ID NO:16 or SEQ ID NO:17 or a fragment thereof.

In still more preferred embodiments, the polypeptide is a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

In the most preferred embodiments, the polypeptide is a sequence as set forth in SEQ ID NO:15, SEQ ID NO:16 and SEQ ID NO:17 or a fragment thereof.

In yet other preferred embodiments of the present invention, in the method of making a hydrolysate, in addition to the isolated polypeptide having proline specific endopeptidase and the polypeptide having tripeptidyl amino peptidase activity a second isolated polypeptide having proline specific endopeptidase activity as described above is added wherein the second isolated polypeptide is different than the isolated polypeptide having proline specific endopeptidase activity.

More preferably, the isolated polypeptide is a polypeptide according to SEQ ID NO:4 or a fragment thereof and the second isolated polypeptide is a polypeptide according to SEQ ID NO:8 or a fragment thereof.

Preferably, the protein substrate is derived from milk. In other preferred embodiments, the protein substrate is derived from wheat.

In another aspect of the present invention, a method for degrading gluten in food is presented having the step of contacting gluten-containing food with an isolated polypeptide having proline specific endopeptidase activity as described above.

Preferably, the food is bread or beer.

In another aspect of the present invention, a method for treating gluten intolerance, celiac disease, dermatitis herpetiformis and/or gluten sensitivity in a patient in need of such treatment, wherein the treatment reduces exposure of said patient to an immunogenic gluten peptide, having the step of orally administering to the patient a therapeutically effective dose of an isolated polypeptide having proline specific endopeptidase activity as described above contemporaneously with the ingestion of a food that may contain gluten.

In another aspect of the present invention, the use is presented of an isolated polypeptide having proline specific endopeptidase activity as described above for the manufacture of a dietary supplement or medicament.

Preferably, the isolated polypeptide having proline specific endopeptidase activity as described above digests gluten fragments that are resistant to normal digestive enzymes.

Preferably, the isolated polypeptide having proline specific endopeptidase activity as described above is admixed with food.

Preferably, the isolated polypeptide having proline specific endopeptidase activity as described above is stable to acid conditions.

In another aspect of the present invention, a formulation is presented having the isolated polypeptide having proline specific endopeptidase activity as described above and a pharmaceutically acceptable excipient.

In other aspect of the present invention, an enzyme blend is presented having a proline specific endopeptidase as described above and a protease wherein the proline specific endopeptidase is different than said protease. Preferably, the protease is selected from the group consisting of a serine protease, a cysteine protease, an endopeptidase, and an exopeptidase.

More preferably, the protease is a serine protease. Still more preferably, the serine protease is a subtilisin.

In other preferred embodiments, the protease is an endopeptidase. Preferably the endopeptidase is a second isolated polypeptide having proline specific endopeptidase activity as described above. More preferably, the isolated polypeptide is a polypeptide according to SEQ ID NO:4 or a fragment thereof and the second isolated polypeptide is a polypeptide according to SEQ ID NO:8 or a fragment thereof.

In other preferred embodiments, the protease is an exopeptidase. Preferably, the exopeptidase is a tripeptidyl aminopeptidase. More preferably, the tripeptidyl aminopeptidase is a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof. Still more preferably, the polypeptide has at least 70% sequence identity to SEQ ID NO:15, SEQ ID NO:16 or SEQ ID NO:17 or a fragment thereof.

In yet more preferred embodiments, the tripeptidyl aminopeptidase is a polypeptide having tripeptidyl aminopeptidase activity having at least 80% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

More preferably, the tripeptidyl aminopeptidase is a polypeptide having tripeptidyl aminopeptidase activity having at least 80% sequence identity to SEQ ID NO:15, SEQ ID NO:16 or SEQ ID NO:17 or a fragment thereof.

In still more preferred embodiments, the tripeptidyl aminopeptidase is a polypeptide having tripeptidyl aminopeptidase activity having at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

More preferably, the tripeptidyl aminopeptidase is a polypeptide having tripeptidyl aminopeptidase activity having at least 90% sequence identity to SEQ ID NO:15, SEQ ID NO:16 or SEQ ID NO:17 or a fragment thereof.

In yet more preferred embodiments, the tripeptidyl aminopeptidase is a polypeptide having tripeptidyl aminopeptidase activity having at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

More preferably, the tripeptidyl aminopeptidase is a polypeptide having tripeptidyl aminopeptidase activity having 95% sequence identity to SEQ ID NO:15, SEQ ID NO:16 or SEQ ID NO:17 or a fragment thereof.

In still more preferred embodiments, the tripeptidyl aminopeptidase is a polypeptide having tripeptidyl aminopeptidase activity having at least 99% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

More preferably, the tripeptidyl aminopeptidase has at least 99% sequence identity to SEQ ID NO:15, SEQ ID NO:16 or SEQ ID NO:17 or a fragment thereof.

In still more preferred embodiments, the tripeptidyl aminopeptidase is a polypeptide having a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

In the most preferred embodiments, the tripeptidyl aminopeptidase is a sequence as set forth in SEQ ID NO:15, SEQ ID NO:16 or SEQ ID NO:17 or a fragment thereof.

In more preferred embodiments where an enzyme blend has a polypeptide having proline specific endopeptidase activity as described above and a tripeptidyl aminopeptidase as described above, a second isolated polypeptide having proline specific endopeptidase activity as describe above is included in the blend wherein the second isolated polypeptide is different than the isolated polypeptide having proline specific endopeptidase activity.

According to this aspect of the present invention, the isolated polypeptide is preferably a polypeptide according to SEQ ID NO:4 or a fragment thereof and the second isolated polypeptide having proline specific endopeptidase activity is a polypeptide according to SEQ ID NO:8 or a fragment thereof.

In another aspect of the present invention, a polynucleotide is presented having a nucleic acid sequence encoding the isolated polypeptide having proline specific endopeptidase activity as described above.

In another aspect of the present invention, a recombinant expression vector is presented having the polynucleotide.

In another aspect of the present invention, a host cell is presented having the recombinant expression vector.

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Example 1

Cloning of MorPro1, AflPro3 and CpoPro1

Three fungal strains (Magnaporthe oryzae 70-15, Aspergillus flavus and Coccidioides posadasii str.C735 delta SOWgp) were selected as potential sources of enzymes which may be useful in various industrial applications. A BLAST search (Altschul et al., J Mol Biol, 215: 403-410, 1990) led to the identification of three genes that encode proteins with homology to a fungal protease: MorPro1 from Magnaporthe oryzae 70-15, AflPro3 from Aspergillus flavus and CpoPro1 from Coccidioides posadasii str.C735 delta SOWgp.

The nucleic acid sequence of full-length MorPro1 gene, as identified from NCBI database (NCBI Reference Sequence: NC_017851.1 from 2214046 to 2215835; complement), is provided in SEQ ID NO: 3. The corresponding full-length protein encoded by the MorPro1 gene is shown in SEQ ID NO: 4 (NCBI Reference Sequence: XP_003716615.1). The nucleic acid sequence of full-length AflPro3 gene, as identified from Broad Institute database (Broad Institute database Reference Sequence: AFL2G_02145), is provided in SEQ ID NO: 5. The corresponding full-length protein encoded by the AflPro3 gene is shown in SEQ ID NO: 6 (NCBI Reference Sequence: XP_002374452.1). The nucleic acid sequence of full-length CpoPro1 gene, as identified from NCBI database (NCBI Reference Sequence: NW_003316003.1 from 2687540 to 2689312; complement), is provided in SEQ ID NO: 7. The corresponding full-length protein encoded by the CpoPro1 gene is shown in SEQ ID NO: 8 (NCBI Reference Sequence: XP_003069863.1).

MorPro1, AflPro3 and CpoPro1 have an N-terminal signal peptide as predicted by SignalP version 4.0 (Nordahl Petersen et al. (2011) Nature Methods, 8:785-786), suggestion that they are all secreted enzymes. The corresponding, predicted, mature enzyme sequence for MorPro1, AflPro3 or CpoPro1 is provided in SEQ ID NO: 9, SEQ ID NO: 10, or SEQ ID NO: 11, respectively.

### Example 2

### Expression of MorPro1, AflPro3 and CpoPro1

The DNA sequence encoding full-length MorPro1 (SEQ ID NO: 4), AflPro3 (SEQ ID NO: 6) or CpoPro1 (SEQ ID NO: 8) was chemically synthesized and inserted into the Trichoderma reesei expression vector pTrex3gM (described in U.S. Published Application 2011/0136197 A1) by Generay (Shanghai, China). The synthesized nucleotide sequences for full-length MorPro1, AflPro3 and CpoPro1 are set forth as SEQ ID NO: 12, 13 and 14, respectively. The pTrex3gM expression vector contained the T. reesei cbh1-derived promoter (cbh1) and cbh1 terminator regions allowing for a strong inducible expression of the gene of interest. The A. nidulans amdS selective marker confer growth of transformants on acetamide as a sole nitrogen source.

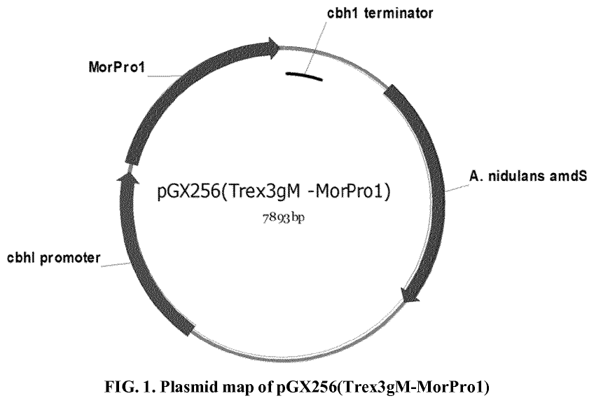

The resulting plasmids were labeled pGX256(Trex3gM-MorPro1), pGX256(Trex3gM-AflPro3) or pGX256(Trex3gM-CpoPro1), respectively. The plasmid map of pGX256(Trex3gM-MorPro1) is provided in FIG. 1 and the other two plasmids have similar composition except for the inserted gene encoding each fungal protease.

Each individual expression plasmid was then transformed into a quad deleted Trichoderma reesei strain (described in WO 05/001036) using biolistic method (Te'o V S et al., J Microbiol Methods, 51:393-9, 2002). Transformants were selected on a medium containing acetamide as a sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies (about 50-100) appeared in about 1 week. After growth on acetamide plates, transformants were picked and transferred individually to acetamide agar plates. After 5 days of growth on acetamide plates, transformants displaying stable morphology were inoculated into 200 μL Glucose/Sophorose defined media in 96-well microtiter plates. The microtiter plate was incubated in an oxygen growth chamber at 28° C. for 5 days. Supernatants from these cultures were used to confirm the protein expression by SDS-PAGE analysis. The stable strain with the highest protein expression was selected and subjected to fermentation in a 250 mL shake flask with Glucose/Sophorose defined media.

To purify MorPro1, AflPro3 and CpoPro1, the crude broth from the shake flask was concentrated using a VivaFlow 200 ultra-filtration device (Sartorius Stedium). Ammonium sulfate was then added to the concentrated solution to a final concentration of 1 M. After filtering, the resulting soluble fraction was applied to a 60 mL Phenyl-FF Sepharose column pre-equilibrated with the loading buffer containing 20 mM Tris-HCl (pH 8.0) and 1 M ammonium sulfate. The corresponding active fractions were pooled, concentrated and subsequently loaded onto a Superdex 75 gel filtration column pre-equilibrated with 20 mM sodium phosphate buffer (pH 7.0) supplemented with additional 0.15 M NaCl and 10% glycerol. The resulting active protein fractions were then pooled and concentrated via the 10K Amicon Ultra devices, and stored in 40% glycerol at −20° C. until usage.

### Example 3

### Proteolytic Activity of MorPro1, AflPro3 and CpoPro1

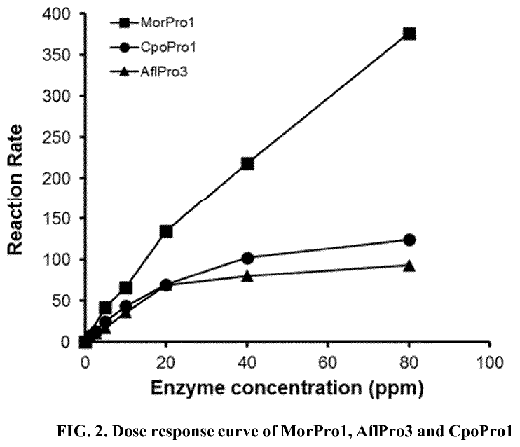

The proteolytic activity of purified MorPro1 or CpoPro1 was measured in 25 mM citrate/phosphate buffer (pH 5), using Succinyl-Ala-Ala-Ala-Pro-paranitroanilide (Suc-AAAP-pNA) (GL Biochem, Shanghai) as the substrate. Prior to the reaction, the enzyme was diluted with water to specific concentrations. The Suc-AAAP-pNA substrate was dissolved in 100% Dimethylsulfoxide (DMSO) to a final concentration of 10 mM. To initiate the reaction, 5 μl of substrate was mixed with 85 μL of citrate/phosphate buffer in a non-binding 96-well Microtiter Plate (96-MTP) (Corning Life Sciences, #3641), and after 5 min pre-incubation at 37° C. in a Thermomixer (Eppendorf), 10 μl of properly diluted purified enzyme (or water as the blank) was added. After sealing the 96-MTP, the reaction was carried out in a Thermomixer at 37° C. and 600 rpm for 10 min, and the absorbance of the resulting solution was measured at 410 nm (A₄₁₀) using a SpectraMax 190. Reaction rate was subsequently calculated (Reaction rate=δA₄₁₀/10 (min)*1000, where δA₄₁₀ is the increase of A₄₁₀ reading within the 10 min incubation time) and plotted against different protein concentrations (from 1.25 ppm to 80 ppm) to demonstrate the proteolytic activity (FIG. 2). Each value was the mean of triplicate assays, with variance less than 5%. The proteolytic assay with Suc-AAAP-pNA as the substrate (shown in FIG. 2) indicates that MorPro1 and CpoPro1 are active proteases.

The proteolytic activity of purified AflPro3 was measured in 25 mM citrate/phosphate buffer (pH 5), using Benzylcarboxy-Glycine-Proline-paranitroanilide (Z-GP-pNA) (Invitrogen, Cat. No. 254295) as the substrate. Prior to the reaction, the enzyme was diluted with water to specific concentrations. The Z-GP-pNA substrate was dissolved in 100% Dimethylsulfoxide (DMSO) to a final concentration of 10 mM. To initiate the reaction, 5 μL of substrate was mixed with 85 μL of citrate/phosphate buffer in a non-binding 96-well Microtiter Plate (96-MTP) (Corning Life Sciences, #3641), and after 5 min pre-incubation at 37° C. in a Thermomixer (Eppendorf), 10 μl of properly diluted purified enzyme (or water as the blank) was added. After sealing the 96-MTP, the reaction was carried out in a Thermomixer at 37° C. and 600 rpm for 30 min, and the absorbance of the resulting solution was measured at 410 nm (A410) using a SpectraMax 190. Reaction rate was subsequently calculated (Reaction rate=δA410/10 (min)*1000, where δA410 is the increase of A410 reading within the 10 min incubation time) and plotted against different protein concentrations (from 1.25 ppm to 80 ppm) to demonstrate the proteolytic activity (FIG. 2). Each value was the mean of triplicate assays, with variance less than 5%. The proteolytic assay with Z-GP-pNA as the substrate (shown in FIG. 2) indicates that AflPro3 is an active protease.

### Example 4

### pH Profile of MorPro1, AflPro3 and CpoPro1

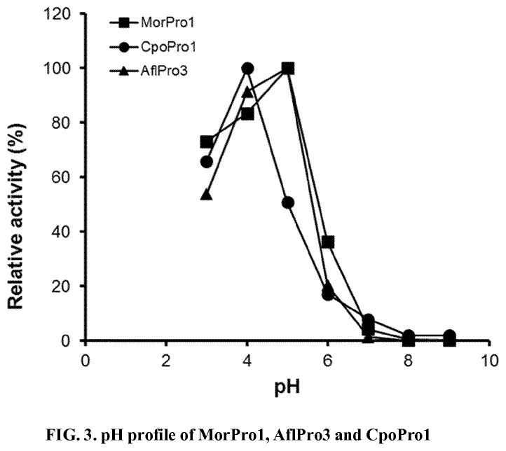

With Suc-AAAP-pNA as the substrate for MorPro1 and CpoPro1, and Z-GP-pNA for AflPro3, the pH profiles of three purified fungal proteases were studied in 16.7 mM citrate/phosphate/CHES buffer with different pH values ranging from 3 to 9. To initiate the assay, 85 μl of citrate/phosphate/CHES buffer with a specific pH was first mixed with 5 μl of 10 mM specific substrate in a 96-MTP and pre-incubated at 37° C. for 5 min, followed by the addition of 10 μl of each water diluted enzyme (100 ppm) (or water alone as the blank control). The reaction was performed and analyzed as described in Example 3. Enzyme activity as each pH was reported as the relatively activity, where the activity at the optimal pH was set to be 100%. The pH vales tested were 3, 4, 5, 6, 7, 8, and 9. Each value was the mean of triplicate assays, with variance less than 5%. As shown in FIG. 3, the optimal pH of MorPro1, AflPro3 and CpoPro1 is 5, 5 and 4, respectively.

### Example 5

Temperature profile of MorPro1, AflPro3 and CpoPro1

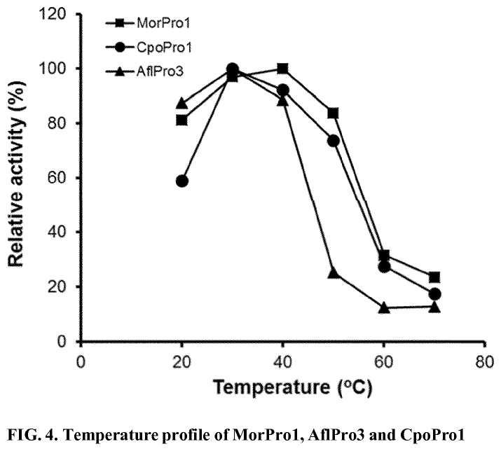

The temperature profiles of three purified fungal proteases were analyzed in 25 mM citrate/phosphate buffer (pH 5) using Suc-AAAP-pNA as the substrate for MorPro1 and CpoPro1, and Z-GP-pNA for AflPro3. The enzyme sample and pNA substrate were prepared as in Example 3. Prior to the reaction, 85 □l of citrate/phosphate buffer and 5 μl of 10 mM pNA substrate were mixed in a 200 □l PCR tube, which was then incubated in a Peltier Thermal Cycler (BioRad) at desired temperatures (i.e. 20˜70° C.) for 5 min. After the incubation, 10 □l of each water diluted enzyme (100 ppm) (or water alone as the blank control) was added to the solution, and the reaction was carried out in the Peltier Thermal Cycle for 10 min at different temperatures. Subsequent absorbance measurements were performed as described in Example 3. The activity was reported as the relative activity, where the activity at the optimal temperature was set to be 100%. The tested temperatures are 20, 30, 40, 50, 60, and 70° C. Each value was the mean of triplicate assays (the value varies no more than 5%). The data in FIG. 4 suggest that MorPro1, AflPro3 and CpoPro1 showed an optimal temperature at 40° C., 30° C. and 30° C., respectively.

### Example 6

### Thermostability of MorPro1, AflPro3 and CpoPro1

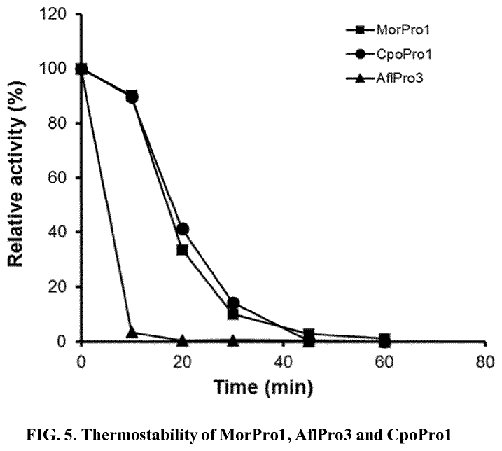

The thermostability analyses of three purified fungal proteases were performed using 50 mM acetate/phosphate buffer (pH 4.5) supplemented with additional 5% (w/w) ethanol as the incubation buffer. For remaining activity measurement, Suc-AAAP-pNA was applied as the substrate for MorPro1 and CpoPro1, while Z-GP-pNA was applied for AflPro3. The purified enzyme was diluted in 1 mL incubation buffer to a final concentration of 1 mg/mL and subsequently incubated at 60° C. for 0, 10, 20, 30, 60 or 90 min. At the end of each incubation period, 100 μL of the enzyme-buffer mixture was transferred to a 96-MTP placed on ice. After the completion of the entire incubation, activity was measured as in Example 3. The activity was reported as the relative activity, where the activity at 0 min incubation time was set to be 100%. Each value was the mean of duplicate assays with variance less than 5%. The result in FIG. 5 shows that after 20 min incubation at 60° C., MorPro1, AflPro3 and CpoPro1 lost 67%, 100% and 60% of its activity, respective. And after 1 hr incubation, all three proteases lost 100% of its activity.

### Example 7

Haze reduction performance of MorPro1, AflPro3 and CpoPro1

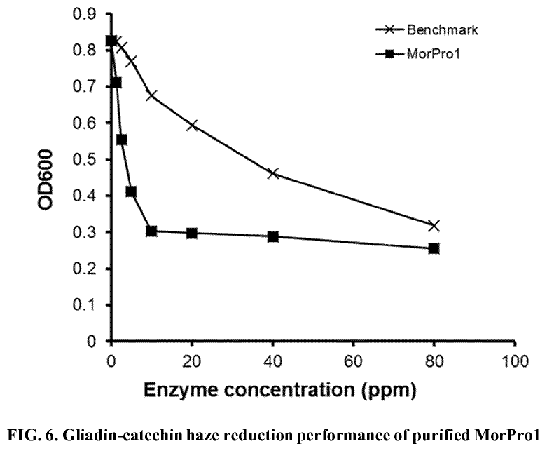

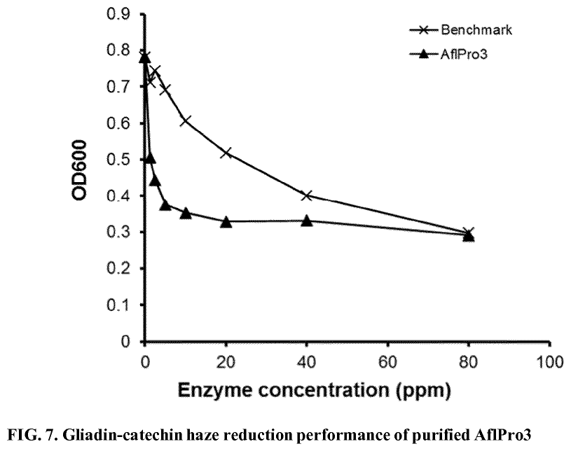

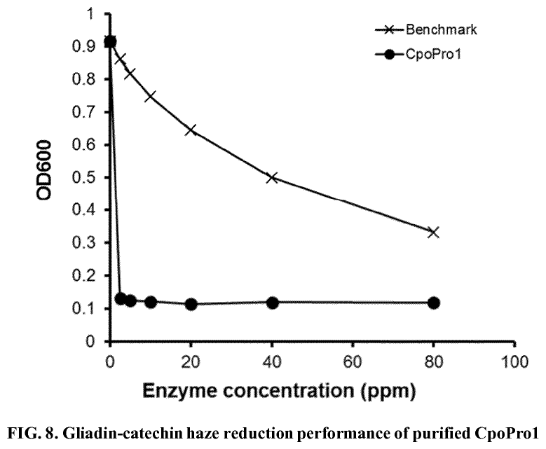

The haze reduction performances of three purified fungal enzymes were evaluated using the gliadin-catechin assay. Prior to the reaction, each enzyme was diluted with water to specific concentrations. And Brewers Clarex® was used as the benchmark. The gliadin substrate (Sigma, Cat. No. G3375) was dissolved in 20 mM acetate/phosphate buffer (pH 4.5) supplemented with additional 0.2% ethanol to a final concentration of 2 mg/mL and the catechin substrate (Sigma, Cat. No. C1251) was dissolved in 20 mM citrate/phosphate buffer (pH 4.5) supplemented with additional 0.2% ethanol to a final concentration of 2 mg/mL. To initiate the assay, 100 μL of gliadin solution was mixed with 5 μL of properly diluted enzyme in a 96-MTP; and after 90 min incubation at 45° C. in a Thermomixer, the resulting 96-MTP was then placed on ice for 5 min, followed by the addition of 100 μl catechin solution. Haze was developed at room temperature for 30 min. The absorbance of the developed haze at 600 nm (A₆₀₀) was measured using a SpectraMax 190 and subsequently plotted against different enzyme concentrations (from 0 to 80 ppm). Each value was the mean of triplicate assays with variance less than 1%. The data in FIGS. 6, 7 and 8 indicate that MorPro1, AflPro3 and CpoPro1 can significantly reduce the gliadin-catechin haze and all of them are more efficient than the benchmark.

### Example 8

### The Performances of MorPro1, AflPro3 and CpoPro1 in Degrading the Immunogenic Gliadin 26-Mer and 33-Mer Peptides

The 26-mer (SEQ ID NO: 1) and 33-mer peptide (SEQ ID NO: 2) test peptides were synthesized by GL Biochem (Shanghai, China).

Prior to the reaction, each purified protease was diluted with water to specific concentrations (20 ppm, 10 ppm or 5 ppm); and each peptide was dissolved in 25 mM Sodium acetate buffer (pH 4.5) to a final concentration of 1 mg/mL. The reaction was initiated by mixing 90 μL of peptide solution with 10 μL of diluted enzyme in a 200 μL PCR tube; and thus the final concentration of the enzyme used in the assay was 2 ppm, 1 ppm or 0.5 ppm. The water diluted Brewers Clarex® or water alone was applied as the Benchmark or blank control, respectively. After 10 min incubation at 40° C. in a Peltier Thermal Cycler (BioRad), the reaction was terminated by heating at 90° C. for 10 min; and 150 μL of the reaction mixture was then filtered via a 96-well 0.22 μm filtration plate (Corning Life Sciences, #3505).

5 μL of the resulting filtrate was subjected to the HPLC analyses using an Agilent 1260 Series HPLC system equipped with a VWD detector. The reaction product was chromatographed on a C18 column (ZORBAX 300SB-C18, 5 μm, 4.6×150 mm, Agilent) at a flow rate of 1 mL min⁻¹ using a gradient from 90% solution A (100% water with 0.1% TFA(v/v))/10% solution B (100% acetonitrile with 0.1% TFA (v/v)) to 60% solution A/40% solution B over 16 min. The substrate peptides are detected by their UV absorbance at 210 nm; and the HPLC retention time for 26-mer or 33-mer is 9.4 min or 13.7 min, respectively. The residual amount of the substrate peptide after enzyme treatment was calculated by comparing its peak area with that of the blank control. The results were summarized in Table 1 and each value was the mean of triplicate assays, with variance less than 5%. As shown in Table 1, after 1 ppm enzyme treatment, the residual amount of 26-mer peptide for MorPro1, AflPro3, CpoPro1 or the Benchmark is 0.07 mg/mL, 0.17 mg/mL, 0.01 mg/mL or 0.46 mg/mL, respectively; while the residue amount of 33-mer peptide for MorPro1, AflPro3, CpoPro1 or the Benchmark is 0.26 mg/mL, 0.01 mg/mL, 0.02 mg/mL or 0.39 mg/mL, respectively. The data suggest that MorPro1, AflPro3 and CpoPro1 are efficient in both peptide degradation.

TABLE 1
Residual amount of 26-mer or 33-mer peptide after protease treatment

| | Residual 26-mer peptide (mg/mL) | | | Residual 33-mer peptide (mg/mL) | | |
|---|---|---|---|---|---|---|
| | 2 ppm | 1 ppm | 0.5 ppm | 2 ppm | 1 ppm | 0.5 ppm |
| Enzyme | protease | protease | protease | protease | protease | protease |
| MorPro1 | 0.01 | 0.07 | 0.24 | 0.12 | 0.26 | 0.44 |
| AflPro3 | 0.05 | 0.17 | 0.38 | 0.01 | 0.01 | 0.09 |
| CpoPro1 | 0.00 | 0.01 | 0.07 | 0.00 | 0.02 | 0.18 |
| Benchmark | 0.20 | 0.46 | 0.65 | 0.18 | 0.39 | 0.58 |

### Example 9

### The Performances of MorPro1, AflPro3 and CpoPro1 in Reducing the Immunogenicity of 26-Mer and 33-Mer Peptides

26-mer immunogenicity assay: Prior to the reaction, each purified protease was diluted with water to a final concentration of 5 ppm; and the 26-mer peptide was dissolved in 25 mM Sodium acetate buffer (pH 4.5) to a final concentration of 1 mg/mL. The reaction was initiated by mixing 45 μL of peptide solution with 5 μL of diluted enzyme in a 200 μL PCR tube. The water diluted Brewers Clarex® or water alone was applied as the Benchmark or blank control, respectively. After 10 min incubation at 40° C. in a Peltier Thermal Cycler (BioRad), the reaction was terminated by heating at 90° C. for 10 min; and the resulting mixture was subjected to the competitive enzyme-linked immunosorbent assays (ELISA) using the RIDASCREEN® Gliadin competitive kit (R-Biopharm, Germany). Following the standard manufacturer's instruction, the absorbance for each specific enzyme assay sample was measured at 450 nm (A₄₅₀) using a SpectraMax 190. The relative A₄₅₀ was then calculated by dividing the A₄₅₀ of the enzyme assay sample by that of the blank control. And the data were subsequently applied to measure the corresponding residual immunogenicity (%) using the standard curve constructed from different concentrations (from 0.0625 mg/mL to 1 mg/mL) of the 26-mer peptide. The results were summarized in Table 2 and each value was the mean of triplicate assays. As shown in Table 2, after enzyme treatment, the residual immunogenicity for MorPro1, AflPro3, CpoPro1 or Benchmark is 61.4%, 68.5%, 38.5% or 99.6%, respectively; indicating that MorPro1, AflPro3 and CpoPro1 are effective in reducing the immunogenicity of the 26-mer peptide.
33-mer immunogenicity assay: Prior to the reaction, each purified protease was diluted with water to a final concentration of 10 ppm; and the 33-mer peptide was dissolved in 25 mM Sodium acetate buffer (pH 4.5) to a final concentration of 2 mg/mL. The reaction was initiated by mixing 45 μL of peptide solution with 5 μL of diluted enzyme in a 200 μL PCR tube. The water diluted Brewers Clarex® or water alone was applied as the Benchmark or blank control, respectively. After 20 min incubation at 40° C. in a Peltier Thermal Cycler (BioRad), the reaction was terminated by heating at 90° C. for 10 min; and the resulting mixture was subjected to the competitive ELISA assays using the RIDASCREEN® Gliadin competitive kit (R-Biopharm, Germany). Following the standard manufacturer's instruction, the absorbance for each specific enzyme assay sample was measured at 450 nm (A₄₅₀) using a SpectraMax 190. The relative A₄₅₀ was then calculated by dividing the A₄₅₀ of the enzyme assay sample by that of the blank control. And the data were subsequently applied to measure the corresponding residual immunogenicity (%) using the standard curve constructed from different concentrations (from 0.25 mg/mL to 2 mg/mL) of the 33-mer peptide. The results were summarized in Table 2 and each value was the mean of sextuplicate assays. As shown in Table 2, after enzyme treatment, the residual immunogenicity for MorPro1, AflPro3, CpoPro1 or Benchmark is 57.5%, 44.6%, 16.9% or 73.7%, respectively; indicating that MorPro1, AflPro3 and CpoPro1 are effective in reducing the immunogenicity of the 33-mer peptide.

TABLE 2
Residual immunogenicity of 26-mer and 33-mer
peptide after protease treatment

| | Residual | Residual |
|---|---|---|
| | immunogenicity of | immunogenicity of |
| Enzyme | 26-mer peptide (%) | 33-mer peptide (%) |
| MorPro1 | 61.4 ± 8.8 | 57.5 ± 7.9 |
| AflPro3 | 68.5 ± 14.7 | 44.6 ± 3.7 |
| CpoPro1 | 38.5 ± 16.2 | 16.9 ± 2.3 |
| Benchmark | 99.6 ± 10.2 | 73.7 ± 4.9 |

### Example 10

### The Performances of MorPro1, AflPro3 and CpoPro1 in Reducing the Immunogenicity of Wheat Gliadins

Prior to the reaction, each purified protease was diluted with water to a final concentration of 10 ppm; and the wheat gliadin (Sigma, Cat. No. G3375) was dissolved in 20 mM citrate/phosphate buffer (pH 4.5) to a final concentration of 25 μg/mL. The reaction was initiated by mixing 45 μL of gliadin solution with 5 μL of diluted enzyme in a 200 μL PCR tube. The water diluted Brewers Clarex® or water alone was applied as the Benchmark or blank control, respectively. After 20 min incubation at 40° C. in a Peltier Thermal Cycler (BioRad), the reaction was terminated by heating at 90° C. for 15 min; and the resulting mixture was subjected to the competitive ELISA assays using the RIDASCREEN® Gliadin competitive kit (R-Biopharm, Germany). Following the standard manufacturer's instruction, the absorbance for each specific enzyme assay sample was measured at 450 nm (A₄₅₀) using a SpectraMax 190. The relative A₄₅₀ was then calculated by dividing the A₄₅₀ of the enzyme assay sample by that of the blank control. And the data were subsequently applied to measure the corresponding residual immunogenicity (%) using the standard curve constructed from different concentrations (from 1.5625 μg/mL to 25 μg/mL) of the wheat gliadin. The results were summarized in Table 3 and each value was the mean of sextuplicate assays. As shown in Table 3, after enzyme treatment, the residual immunogenicity for MorPro1, AflPro3, CpoPro1 or Benchmark is 18.7%, 88.3%, 53.6% or 59.7%, respectively; indicating that all three proteases are capable of reducing the wheat gliadin immunogenicity.

TABLE 2
Residual immunogenicity of wheat gliadin after
protease treatment

| Enzyme | Residual Immunogenicity (%) |
|---|---|
| MorPro1 | 18.7 ± 3.2 |
| AflPro3 | 88.3 ± 10.2 |
| CpoPro1 | 53.6 ± 7.7 |
| Benchmark | 59.7 ± 8.3 |

### Example 11

### Chill Haze Reduction Performance of AflPro3, CpoPro1 and MorPro1 in Test Tubes

The haze reduction performance of AflPro3, CpoPro1 and MorPro1 was evaluated in a Pilsener beer (from Research Brewery St. Johann brewed on 100% Pilsener malt (Fuglsang, Denmark; batch 19.03.2015.) brewed without the use of fining agents). The enzyme samples were added to 8 ml beer in 10 ml glass tubes (0.5, 2.5 and 5 ppm final enzyme protein concentrations and 0 ppm as blank control) for evaluation of haze effects. The tubes were kept at 20° C. in the dark. Right after enzyme addition (day 0), after 24 hours (day 1) and on day 4 tubes were chilled for 1 hour on ice and measured according to the standard method EBC 9.29 (Analytica-EBC (1997): Method 9.29, Haze in beer: calibration of haze meters) using a Turbimeter, Hach 2100AN. The data from haze measurements shown in Table 11.1 indicates that AflPro3, CpoPro1 and MorPro1 can reduce haze strongly. The lowest dosage (0.5 ppm) of AflPro3, CpoPro1 and MorPro1 reduces haze over time from day 1 to day 4, whereas when dosed at 2.5 and 5 ppm the enzymes give the full haze removal or a very marked effect already on day 1.

TABLE 11.1
Haze reduction in test tubes

| | Dosage | | | |
|---|---|---|---|---|
| Treatment | (ppm) | DAY 0 | DAY 1 | DAY 4 |
| Control | 0 | 24 | 24 | 23 |
| MorPro 1 | 0.5 | 25 | 11 | 6 |
| | 2.5 | 25 | 12 | 8 |
| | 5 | 25 | 6 | 8 |
| AflPro 3 | 0.5 | 25 | 23 | 20 |
| | 2.5 | 25 | 20 | 8 |
| | 5 | 24 | 13 | 8 |
| CpoPro 1 | 0.5 | 24 | 18 | 10 |
| | 2.5 | 24 | 11 | 8 |
| | 5 | 25 | 7 | 11 |

### Example 12

### Haze Reduction Performance of AflPro3, CpoPro1 and MorPro1 in Beer Bottles

Furthermore, the haze reduction performance of AflPro3, CpoPro1 and MorPro1 was evaluated in a Pilsener beer (from Research Brewery St. Johann brewed on 100% Pilsener malt (Fuglsang, Denmark; batch 19.03.2015.) and brewed without the use of fining agents). The enzyme samples (at 2.5 ppm enzyme protein concentrations together with 0 ppm as blank control) were added to 330 ml beer bottles, which were kept for 5 days at 20° C. in the dark. Chill haze was measured after 24 h at 0° C., whereas accelerated haze was measured after an incubation schedule of 24 h at 0° C., 48 h at 60° C. and 24 h at 0° C. Haze development within the bottles was measured at 0° C. using a SIGRIST Lab Scat 2, instrument (from SIGRIST-PHOTOMETER AG, Ennetburgen, Switzerland) with a 25° or 90° angle in EBC units as described by method EBC 9.29 (Analytica-EBC (1997): Method 9.29, Haze in beer: calibration of haze meters). With all enzyme candidates strong reductions in chill haze as well as accelerated haze were measured at 25° and 90° when compared to haze in the control (Table 12.1). This indicates that the candidates are highly effective in haze reduction in beer.

TABLE 12.1
Haze in Pilsener beer treated without or with 2.5 ppm AflPro1,
AflPro3, TrePro1, CpoPro1 and MorPro1 (measured in EBC units)

| | Chill haze | Chill haze | Accelerated | Accelerated |
|---|---|---|---|---|
| | 25° | 90° | haze 25° | haze 90° |
| Control | 17.0 | 13.2 | 23.2 | 16.3 |
| AflPro3 | 1.5 | 5.6 | 2.7 | 8.7 |
| CpoPro1 | 0.9 | 3.9 | 1.2 | 5.6 |
| MorPro1 | 1.0 | 4.5 | 1.4 | 6.0 |

### Example 13

### Thermoinactivation of AflPro3 and CpoPro1 During Beer Pasteurization

In order to evaluate the inactivation of AflPro3 and CpoPro1 during beer pasteurisation the enzymes were incubated in Heineken beer with and without a heat treatment for 25 seconds at 75° C. The enzyme activity before and after the heat treatment was measured in an fluorometric assay using Z-Gly-Pro-AMC. As a control papain was also tested and measured before and after heat treatment with Z-Phe-Arg-AMC substrate.

The substrate used were Z-Gly-Pro-AMC (I-1145; BACHEM) or for papain Z-Phe-Arg-AMC (I1160; BACHEM). A 10 mM substrate stock solution in DMSO was prepared. A 0.1 mM working substrate solution was prepared by adding 5 ul of substrate stock solution to 495 ul of buffer (0.1 M Mcllvain buffer, pH 5.0). For the assay 50 μl buffer and 25 μl 0.1 mM working substrate solution and 25 μl of enzyme sample diluted in buffer was used.
96 well plates (no. 265301; Thermo Scientific) were used for the assay and incubated at 37° C. and are read over time in a SpectraMax Gemini Microplate Spectrofluorometer using excitation at 355 nm and emission at 460 nm with a cutoff at 455 nm.

TABLE 13.1
Residual activity after pasteurization in beer measured
with the substrates indicated

| Enzyme | Papain | AflPro3 | CpoPro1 |
|---|---|---|---|
| Substrate | Z-Phe-Arg- | Z-Gly-Pro- | Z-Gly-Pro- |
| | AMC | AMC | AMC |
| Residual | 87 | 8 | 0 |
| activity (%) | | | |

As seen in Table 13.1 AflPro3 shows only 8% and CpoPro1 no residual activity in contrast to papain which has 87% remaining activity after heat treatment. This indicates that AflPro3 and CpoPro1 will be highly or totally inactivated during beer pasteurization in contrast to papain.

### Example 14

Foam Stability of Beer Treated with AflPro3, CpoPro1 and MorPro1

The effect of AflPro3, CpoPro1 and MorPro1 on foam stability was evaluated in a Pilsener beer (from Research Brewery St. Johann brewed on 100% Pilsener malt (Fuglsang, Denmark; batch 19.03.2015.) and brewed without the use of fining agents). The enzyme samples (at 2.5 ppm enzyme protein concentrations together with 0 ppm as blank control) were added to 330 ml beer bottles, which were kept for 5 days at 20° C. in the dark. Foam stability was measured using a NIBEM-T Meter according to procedure EBC 9.42 (European Brewing Convention Analytica-EBC section 9 Beer, Method 9.42 Foam Stability of beer using the NIBEM-T Meter). The beer is applied with carbon dioxide gas at a pressure of 2 bar and immediately the foam collapse time (FCT) is measured for 10, 20 and 30 mm reduction of foam. As shown in Table 14.1 treatment of beer with AflPro3, CpoPro1 and MorPro1 does not reduce FCT compared to control, on the contrary FCT is increased, meaning that foam stability is improved.

TABLE 14.1
Foam collapse time (FCT) in beer treated
MorPro1, AflPro3 and CpoPro1

| FCT (sec) | Control | MorPro1 | AflPro3 | CpoPro1 |
|---|---|---|---|---|
| NIBEM 10 | 75 | 82 | 84 | 79 |
| NIBEM 20 | 148 | 162 | 168 | 157 |
| NIBEM 30 | 225 | 243 | 250 | 232 |

Although the foregoing invention has been described in some detail by way of illustration and example, for purposes of clarity of understanding, certain changes and modifications can be practiced within the scope of the appended claims. In addition, each reference provided herein is incorporated by reference in its entirety for all purposes to the same extent as if each reference was individually incorporated by reference. To the extent the content of any citation, including website or accession number may change with time, the version in effect at the filing date of this application is meant. Unless otherwise apparent from the context any step, element, aspect, feature of embodiment can be used in combination with any other.

What is claimed is:

1. An isolated polypeptide having proline specific endopeptidase activity comprising a polypeptide having at least 70% sequence identity to one of SEQ ID NO: 4, SEQ ID NO: 6 and SEQ ID NO: 8 or a fragment thereof.

2. An isolated polypeptide according to claim 1 wherein the polypeptide has at least 80% sequence identity to one of SEQ ID NO: 4, SEQ ID NO: 6 and SEQ ID NO: 8 or a fragment thereof.

3. An isolated polypeptide according to claim 2 wherein the polypeptide has at least 90% sequence identity to one of SEQ ID NO: 4, SEQ ID NO: 6 and SEQ ID NO: 8 or a fragment thereof.

4. An isolated polypeptide according to claim 3 wherein the polypeptide has at least 95% sequence identity to one of SEQ ID NO: 4, SEQ ID NO:6 and SEQ ID NO:8 or a fragment thereof.

5. An isolated polypeptide according to claim 4 wherein the polypeptide has at least 99% sequence identity to one of SEQ ID NO: 4, SEQ ID NO:6 and SEQ ID NO:8.

6. An isolated polypeptide according to claim 5 wherein the polypeptide comprises a sequence according to one of SEQ ID NO:4, SEQ ID NO:6 and SEQ ID NO:8 or a fragment thereof.

7. A method for the reduction or prevention of haze in a beverage comprising adding an isolated polypeptide having proline specific endopeptidase activity according to any of claims 1-6 to said beverage.

8. A method according to claim 7 wherein the beverage contains at least one protein.

9. A method according to claim 8 wherein the protein comprises hordein.

10. A method according to claim 8 wherein the beverage further comprises polyphenols.

11. A method according to claim 7 wherein the beverage has a pH of less than 7.

12. A method according to claim 7 wherein the beverage is a fruit juice.

13. A method according to claim 7 wherein the beverage is a wine.

14. A method according to claim 7 wherein the beverage is a beer.

15. A method according to claim 14 wherein the isolated polypeptide is added to a mash.

16. A method according to claim 14 wherein the isolated polypeptide is added before haze formation.

17. A method according to claim 14 wherein the isolated polypeptide is added after haze formation.

18. A method according to any of claims 7 to 17 further comprising adding a second isolated polypeptide having proline specific endopeptidase activity according to any of claims 1 to 6 wherein the second isolated polypeptide is different than said isolated polypeptide.

19. A method according to claim 18 wherein the isolated polypeptide comprises a polypeptide comprising SEQ ID NO:4 or a fragment thereof and the second isolated polypeptide comprises a polypeptide comprising SEQ ID NO:8 or a fragment thereof.

20. A method for forming a protein hydrolysate comprising adding to a protein substrate an isolated polypeptide having endopeptidase according to any of claims 1-6.

21. A method for forming a protein hydrolysate according to claim 20 further comprising adding a protease wherein said protease is different than said isolated polypeptide.

22. A method according to claim 21 wherein the protease is selected from the group consisting of a serine protease, a cysteine protease, an endopeptidase, and an exopeptidase.

23. A method according to claim 22 wherein the protease is a serine protease

24. A method according to claim 23 wherein the serine protease is a subtilisin.

25. A method according to claim 22 wherein the protease is an endopeptidase.

26. A method according to claim 25 wherein the endopeptidase is a second isolated polypeptide having proline specific endopeptidase activity according to any of claims 1 to 6.

27. A method according to claim 22 wherein the isolated polypeptide comprises a polypeptide comprising SEQ ID NO:4 or a fragment thereof and the second isolated polypeptide comprises a polypeptide comprising SEQ ID NO:8 or a fragment thereof.

28. A method according to claim 22 wherein said protease is an exopeptidase.

29. A method according to claim 28 wherein said exopeptidase is a tripeptidyl aminopeptidase.

30. A method for forming a protein hydrolysate according to claim 29 wherein said tripeptidyl aminopeptidase comprises a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

31. A method for forming a protein hydrolysate according to claim 30 wherein the polypeptide has at least 70% sequence identity to SEQ ID NO:15, SEQ ID NO:16 or SEQ ID NO:17 or a fragment thereof.

32. A method for forming a protein hydrolysate according to claim 30 wherein said polypeptide has at least 80% sequence identity to SEQ ID NO:15, SEQ ID NO:16, SEQ NO:17, SEQ ID NO:18, SEQ ID NO:19, SEQ NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

33. A method for forming a protein hydrolysate according to claim 32 wherein the polypeptide has at least 80% sequence identity to SEQ ID NO:15, SEQ ID NO:16 or SEQ ID NO:17 or a fragment thereof.

34. A method for forming a protein hydrolysate according to claim 32 wherein said polypeptide has at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

35. A method for forming a protein hydrolysate according to claim 34 wherein the polypeptide has at least 90% sequence identity to SEQ ID NO:15, SEQ ID NO:16 or SEQ ID NO:17 or a fragment thereof.

36. A method for forming a protein hydrolysate according to claim 34 wherein said polypeptide has at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

37. A method for forming a protein hydrolysate according to claim 36 wherein the polypeptide has at least 95% sequence identity to SEQ ID NO:15, SEQ ID NO:16 or SEQ ID NO:17 or a fragment thereof.

38. A method for forming a protein hydrolysate according to claim 36 wherein said polypeptide has at least 99% sequence identity to SEQ ID NO:15, SEQ ID NO:16, SEQ NO:17, SEQ ID NO:18, SEQ ID NO:19, SEQ NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

39. A method for forming a protein hydrolysate according to claim 38 wherein the tripeptidyl aminopeptidase has at least 99% sequence identity to SEQ ID NO:15, SEQ ID NO:16 or SEQ ID NO:17 or a fragment thereof.

40. A method for forming a protein hydrolysate according to claim 38 wherein said tripeptidyl aminopeptidase comprises a polypeptide comprising a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

41. A method for forming a hydrolysate according to claim 40 wherein the tripeptidyl aminopeptidase comprises a sequence as set forth in SEQ ID NO:15, SEQ ID NO:16 or SEQ ID NO:17 or a fragment thereof.

42. A method for forming a hydrolysate according to any of claims 20-41 further comprising adding a second isolated polypeptide having proline specific endopeptidase activity according to any of claims 1 to 6 wherein the second isolated polypeptide is different than said isolated polypeptide having proline specific endopeptidase activity.

43. A method according to claim 42 wherein the isolated polypeptide comprises a polypeptide comprising SEQ ID NO:4 or a fragment thereof and the second isolated polypeptide comprises a polypeptide comprising SEQ ID NO:8 or a fragment thereof.

44. A method for forming a protein hydrolysate according to any of claims 20 to 43 wherein the protein substrate is derived from milk.

45. A method for forming a protein hydrolysate according to any of claims 20 to 43 wherein the protein substrate is derived from wheat.

46. A method for degrading gluten in food, said method comprising contacting gluten-containing food with an endopeptidase according to any of claims 1 to 6.

47. A method according to claim 46 wherein the food is bread or beer.

48. A method for treating gluten intolerance, celiac disease, dermatitis herpetiformis and/or gluten sensitivity in a patient in need of such treatment, wherein said treatment reduces exposure of said patient to an immunogenic gluten peptide, said method comprising the step of orally administering to said patient a therapeutically effective dose of an isolated polypeptide of any one of claims 1 to 6 contemporaneously with the ingestion of a food that may contain gluten.

49. Use of an isolated polypeptide according to any of claims 1 to 6 for the manufacture of a dietary supplement or medicament.

50. An isolated polypeptide according to any of claims 1 to 6 wherein said isolated polypeptide digests gluten fragments that are resistant to normal digestive enzymes.

51. An isolated polypeptide according to any of claims 1 to 6 wherein said isolated polypeptide is admixed with food.

52. An isolated polypeptide according to any of claims 1 to 6 wherein said isolated polypeptide is stable to acid conditions.

53. A formulation comprising an isolated polypeptide according to any of claims 1 to 6 and a pharmaceutically acceptable excipient.

54. An enzyme blend comprising a proline specific endopeptidase according to any of claims 1 to 6 and a protease wherein said proline specific endopeptidase is different than said protease.

55. An enzyme blend according to claim 54 wherein the protease is selected from the group consisting of a serine protease, a cysteine protease, an endopeptidase, and an exopeptidase.

56. An enzyme blend according to claim 55 wherein the protease is a serine protease

57. An enzyme blend according to claim 56 wherein the serine protease is a subtilisin.

58. An enzyme blend according to claim 55 wherein the protease is an endopeptidase.

59. An enzyme blend according to claim 58 wherein the endopeptidase is a second isolated polypeptide having proline specific endopeptidase activity according to any of claims 1 to 6.

60. An enzyme blend according to claim 59 wherein the isolated polypeptide comprises a polypeptide comprising SEQ ID NO:4 or a fragment thereof and the second isolated polypeptide comprises a polypeptide comprising SEQ ID NO:8 or a fragment thereof.

61. An enzyme blend according to claim 55 wherein said protease is an exopeptidase.

62. An enzyme blend according to claim 61 wherein said exopeptidase is a tripeptidyl aminopeptidase.

63. An enzyme blend according to claim 62 wherein said tripeptidyl aminopeptidase comprises a polypeptide having tripeptidyl aminopeptidase activity having at least 70% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

64. An enzyme blend according to claim 63 wherein the polypeptide has at least 70% sequence identity to SEQ ID NO:15, SEQ ID NO:16 or SEQ ID NO:17 or a fragment thereof.

65. An enzyme blend according to claim 63 wherein said polypeptide has at least 80% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

66. An enzyme blend according to claim 65 wherein the polypeptide has at least 80% sequence identity to SEQ ID NO:15, SEQ ID NO:16 or SEQ ID NO:17 or a fragment thereof.

67. An enzyme blend according to claim 65 wherein said polypeptide has at least 90% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

68. An enzyme blend according to claim 67 wherein the polypeptide has at least 90% sequence identity to SEQ ID NO:15, SEQ ID NO:16 or SEQ ID NO:17 or a fragment thereof.

69. An enzyme blend according to claim 67 wherein said polypeptide has at least 95% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

70. An enzyme blend according to claim 69 wherein the polypeptide has at least 95% sequence identity to SEQ ID NO:15, SEQ ID NO:16 or SEQ ID NO:17 or a fragment thereof.

71. An enzyme blend according to claim 69 wherein said polypeptide has at least 99% sequence identity to SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

72. An enzyme blend according to claim 71 wherein the tripeptidyl aminopeptidase has at least 99% sequence identity to SEQ ID NO:15, SEQ ID NO:16 or SEQ ID NO:17 or a fragment thereof.

73. An enzyme blend according to claim 71 wherein said tripeptidyl aminopeptidase comprises a polypeptide comprising a sequence as set forth in SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO: 68 or SEQ ID NO: 69 or a fragment thereof.

74. An enzyme blend according to claim 73 wherein the tripeptidyl aminopeptidase comprises a sequence as set forth in SEQ ID NO:15, SEQ ID NO:16 or SEQ ID NO:17 or a fragment thereof.

75. An enzyme blend according to any of claims 58-74 further comprising adding a second isolated polypeptide having proline specific endopeptidase activity according to any of claims 1 to 6 wherein the second isolated polypeptide is different than said isolated polypeptide having proline specific endopeptidase activity.

76. An enzyme blend according to claim 75 wherein the isolated polypeptide comprises a polypeptide comprising SEQ ID NO:4 or a fragment thereof and the second isolated polypeptide comprises a polypeptide comprising SEQ ID NO:8 or a fragment thereof.

77. A polynucleotide comprising a nucleic acid sequence encoding the endopeptidase of any one of claims 1 to 6.

78. A recombinant expression vector comprising a polynucleotide according to claim 77.

79. A host cell comprising the recombinant expression vector according to claim 78.
